SEQUENCE LISTING (10 sequences)

SEQ ID NO: 1 - DNA, 1659 bp, Artificial Sequence
  Synthetic
atggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - DNA, 4011 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 6832 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 4 - DNA, 6 bp, Artificial Sequence
  Synthetic
gccrcc

SEQ ID NO: 5 - protein, 552 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 6 - protein, 127 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 7 - protein, 195 aa, Artificial Sequence
  Synthetic
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPP

SEQ ID NO: 8 - protein, 322 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPP

SEQ ID NO: 9 - protein, 357 aa, Artificial Sequence
  Synthetic
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 10 - protein, 425 aa, Artificial Sequence
  Synthetic
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

(19) United States
(12) Patent Application Publication
(10) Pub. No.: US 2023/0048017 A1
(43) Pub. Date: Feb. 16, 2023

(54) ADENO-ASSOCIATED VIRUS FOR DELIVERY OF KH902 (CONBERCEPT) AND USES THEREOF

(71) Applicant: University of Massachusetts, Boston, MA (US)

(72) Inventors: Guangping Gao, Westborough, MA (US); Phillip Tai, Worcester, MA (US); Claudio Punzo, Brookline, MA (US); Haijiang Lin, Newton, MA (US)

(73) Assignee: University of Massachusetts, Boston, MA (US)

(21) Appl. No.: 17/685,457

(22) Filed: Mar. 3, 2022

Related U.S. Application Data
(63) Continuation of application No. PCT/US2021/048917, filed on Sep. 2, 2021.
Application No. PCT/US2021/048917 is a continuation-in-part of application No. PCT/US2020/049243, filed on Sep. 3, 2020.
(60) Provisional application No. 63/179,700, filed on Apr. 26, 2021.
(60) Provisional application No. 63/074,361, filed on Sep. 3, 2020.
(60) Provisional application No. 62/895,546, filed on Sep. 4, 2019.

(51) Int. Cl. C12N 15/86; C07K 14/005; C12N 7/00; A61K 35/76; A61P 9/00

(52) U.S. Cl. CPC C12N 15/86; C07K 14/005; C12N 7/00; A61K 35/76; A61P 9/00

(57) ABSTRACT

Aspects of the disclosure relate to compositions and methods for expressing anti-Vascular endothelial cell growth factor (VEGF) agent in a cell or subject. In some embodiments, the disclosure provides rAAVs comprising a capsid protein (e.g., AAV2 variants, AAV2/3 hybrid variants, AAV8 variants, etc.), and a transgene encoding an anti-VEGF agent (e.g., KH902) and one or more regulatory sequences. In some embodiments, compositions described herein are useful for treating subjects having diseases associated with angiogenesis or aberrant VEGF activity/signaling.

### RELATED APPLICATIONS

This application is a continuation in part of International Patent Application Serial No. PCT/US2020/049243, filed on Sep. 3, 2020, which claims the benefit under 35 U.S.C. 119(e) of the filing date of U.S. provisional Application Ser. No. 62/895,546, filed Sep. 4, 2019, entitled “ADENO ASSOCIATED VIRUS VECTOR PLATFORM FOR DELIVERY OF KH902 (CONBERCEPT) AND USES THEREOF,” the entire contents of each of which are incorporated by reference herein.

This application is also a continuation of International Patent Application Serial No. PCT/US2021/048917, filed Sep. 2, 2021, which claims the benefit under 35 U.S.C. 119(e) of the filing date of U.S. provisional Application Ser. No. 63/179,700, filed Apr. 26, 2021, entitled “ADENO-ASSOCIATED VIRUS FOR DELIVERY OF KH902 (CONBERCEPT) AND USES THEREOF”, and U.S. provisional Application Ser. No. 63/074,361, filed Sep. 3, 2020, entitled “ADENO-ASSOCIATED VIRUS FOR DELIVERY OF KH902 (CONBERCEPT) AND USES THEREOF”, the entire contents of each of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 28, 2022 is named U0120.70125US02-SEQ-LJG and is 34,801 bytes in size.

### BACKGROUND

KH902 is a vascular endothelial growth factor (VEGF) receptor fusion protein currently undergoing clinical trials for anti-VEGF treatment. Current challenges in anti-VEGF therapy include the need for repeated injections to sustain efficacy and long-acting formulations of anti-VEGF drugs. Therefore, there is need for development of novel methods for long-term delivery of anti-VEGF agent into targeted cells and/or tissues.

### SUMMARY

Aspects of the disclosure relate to compositions and methods for delivery of anti-VEGF agent (e.g., KH902) to cells and/or tissues (e.g., cells of a subject). The disclosure is based, in part, rAAVs engineered to express a transgene encoding an anti-VEGF agent (e.g., KH902).

In some aspects, the present disclosure provides a recombinant adeno-associated virus (rAAV), comprising: (i) an AAV capsid protein, wherein the capsid protein is a variant of AAV2 capsid protein, an AAV2/3 hybrid capsid protein, and/or AAV8 capsid protein; and (ii) an isolated nucleic acid comprising a transgene encoding an anti-vascular endothelial growth factor (anti-VEGF) agent, the transgene being flanked by adeno-associated virus (AAV) inverted terminal repeats (ITRs).

In some embodiments, the anti-VEGF agent is a human VEGF decoy receptor. In some embodiments, the human VEGF decoy receptor comprises extracellular domain 2 of human VEGF receptor 1. In some embodiments, the human VEGF decoy receptor comprises extracellular domains 3 and 4 of human VEGF receptor 2. In some embodiments, the VEGF decoy receptor is capable of binding to anti-vascular endothelial growth factor (VEGF) and/or placenta growth factor (PlGF).

In some embodiments, the anti-VEGF agent is a human VEGF receptor fusion protein. In some embodiments, the human VEGF receptor fusion protein comprises the extracellular domain 2 of human VEGF receptor 1 fused to the extracellular domain 3 and 4 of human VEGF receptor 2. In some embodiments, the human VEGF receptor fusion protein comprises the extracellular domain 2 of human VEGF receptor 1 fused to an Fc portion of an immunoglobulin. In some embodiments, the human VEGF receptor fusion protein comprises the extracellular domain 3 and 4 of human VEGF receptor 2 fused to an Fc portion of an immunoglobulin. In some embodiments, the human VEGF receptor fusion protein comprises the extracellular domain 2 of human VEGF receptor 1 fused to the extracellular domain 3 and 4 of human VEGF receptor 2, and further fused to an Fc portion of an immunoglobulin. In some embodiments, the anti-VEGF agent is KH902. In some embodiments, the anti-VEGF agent comprises an amino acid sequence at least 50%, at least 60%, at least 70%, at least 80%, 90%, 99% or 100% identical to amino acid sequence of SEQ ID NO: 5, or a portion thereof. In some embodiments, the transgene comprises a nucleic acid sequence at least 50%, at least 60%, at least 70%, at least 80%, 90%, 99% or 100% identical to nucleic acid sequence of SEQ ID NO: 1 or a codon optimized variant thereof. In some embodiments, the anti-VEGF agent is capable of binding to anti-vascular endothelial growth factor (VEGF) and/or placenta growth factor (PlGF).

In some embodiments, the isolated nucleic acid further comprises a promoter operably linked to the transgene. In some embodiments, the promoter comprises a cytomegalovirus (CMV) early enhancer. In some embodiments, the promoter is a chimeric cytomegalovirus (CMV)/Chicken β-actin (CB) promoter. In some embodiments, the transgene comprises one or more introns. In some embodiments, at least one intron is positioned between the promoter and the nucleic acid sequence encoding the anti-vascular endothelial growth factor (anti-VEGF) agent.

In some embodiments, the transgene comprises a Kozak sequence. n some embodiments, the Kozak sequence is positioned between the intron and the transgene encoding the anti-vascular endothelial growth factor (anti-VEGF) agent.

In some embodiments, the transgene comprises a 3′ untranslated region (3′UTR). n some embodiments, the transgene further comprises one or more miRNA binding sites. n some embodiments, the one or more miRNA binding sites are positioned in a 3′UTR of the transgene. n some embodiments, the at least one miRNA binding site is an immune cell-associated miRNA binding site. n some embodiments, the immune cell-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181 a, miR-223 and miR-424, miR-221, miR-222, let-7i, miR-148, and miR-152.

In some embodiments, the ITRs are adeno-associated virus ITRs of a serotype selected from the group consisting of AAV1 ITR, AAV2 ITR, AAV3 ITR, AAV4 ITR, AAV5 ITR, and AAV6 ITR.

In some embodiments, the isolated nucleic acid comprises a nucleic acid sequence at least 80%, 90%, 99% or 100% identical to the nucleic acid sequence of SEQ ID NO: 2.

In some embodiments, the capsid protein comprises an amino acid sequence at least 50%, at least 60%, at least 70%, at least 80%, at least 90%, at least 95%, at least 99% or 100% identical to amino acid sequences of v224 capsid protein, v326 capsid protein, v358 capsid protein, v46 capsid protein, v56 capsid protein, v66 capsid protein, v67 capsid protein, v81 capsid protein, v439 capsid protein, v453 capsid protein, v513 capsid protein, v551 capsid protein, v556 capsid protein, v562 capsid protein, or v598 capsid protein. In some embodiments, the capsid protein comprises an amino acid sequence at least 50%, at least 60%, at least 70%, at least 80%, at least 90%, at least 95%, at least 99% or 100% identical to amino acid sequences of v224 capsid protein, v326 capsid protein, or v56 capsid protein. In some embodiments, the capsid protein has tropism for ocular tissue. In some embodiments, the ocular tissue comprises ocular neurons, retina, sclera, choroid, retina, vitreous body, macula, fovea, optic disc, lens, pupil, iris, aqueous fluid, cornea, conjunctiva ciliary body, or optic nerve.

In some embodiments, the rAAV is a single-stranded AAV (ssAAV) or a self-complementary AAV (scAAV).

In some embodiments, capsid protein variants is capable of increasing rAAV packaging efficiency as compared to the wild-type capsid protein they derive from. In some embodiments, the AAV2 capsid protein variant is capable of increasing rAAV packaging efficiency by at least 1-fold, 2-fold, 3-fold, 4-fold, 5-fold, 6-fold, 7-fold, 8-fold, 9-fold, 10-fold or more as compared to the wild-type AAV2 capsid protein. In some embodiments, the AAV2/3 hybrid capsid protein variant is capable of increasing rAAV packaging efficiency by at least 1-fold, 2-fold, 3-fold, 4-fold, 5-fold, 6-fold, 7-fold, 8-fold, 9-fold, 10-fold or more as compared to the wild-type AAV3b capsid protein.

In some aspects, the present disclosure provides a recombinant adeno-associated virus comprising: (i) a rAAV capsid protein, wherein the capsid protein is a variant of AAV8 capsid protein, AAV2 capsid protein and/or an AAV2/3 hybrid capsid protein; and (ii) a recombinant adeno-associated virus (rAAV) vector comprising a nucleic acid comprising, in 5′ to 3′ order: (a) a 5′ AAV ITR; (b) a CMV enhancer; (c) a CBA promoter; (d) a chicken beta-actin intron; (e) a Kozak sequence; (f) a transgene encoding an anti-VEGF agent, wherein the anti-VEGF agent is encoded by the nucleic acid sequence in SEQ ID NO: 1; (g) a rabbit beta-globin polyA signal tail; and (h) a 3′ AAV ITR.

In some aspects, the present disclosure provides a host cell comprising the rAAV as described herein. In some embodiments, the host cell is a mammalian cell, yeast cell, bacterial cell, or insect cell.

In some aspects, the present disclosure provides a pharmaceutical composition comprising the rAAV as described herein. In some embodiments, the pharmaceutical composition further comprises a pharmaceutically acceptable carrier. In some embodiments, the pharmaceutical composition is formulated for intravitreal injection, intravenous injection, intratumoral injection, or intramuscular injection.

In some aspects, the present disclosure provides a method of inhibiting VEGF or PlGF activity in a subject in need thereof, the method comprising administering to the subject an effective amount of the rAAV, or the pharmaceutical composition as described herein.

In some aspects, the present disclosure provides a method of delivering an anti-VEGF agent in a subject in need thereof, the method comprising administering to the subject an effective amount of the rAAV or the pharmaceutical composition as described herein.

In some aspects, the present disclosure provides a method of treating a neovascularization associated disease, an angiogenesis associated disease, or a VEGF associated disease in a subject in need thereof, the method comprising administering to the subject an effective amount of the rAAV or the pharmaceutical composition as described herein.

In some aspects, the disclosure provides an rAAV, or a composition comprising the rAAV for use in inhibiting VEGF activity in a subject in need thereof, wherein the rAAV comprises an adeno-associated virus (AAV) capsid protein (e.g., AAV 2 variants or AAV2/3 hybrid variants) and an isolated nucleic acid comprising a transgene encoding an anti-VEGF agent (e.g., KH902).

In some aspects, the disclosure provides an rAAV, or a composition comprising the rAAV for use in delivering an anti-VEGF agent in a subject in need thereof, wherein the rAAV comprises an adeno-associated virus (AAV) capsid protein (e.g., AAV 2 variants or AAV2/3 hybrid variants) and an isolated nucleic acid comprising a transgene encoding an anti-VEGF agent (e.g., KH902).

In some aspects, the disclosure provides an rAAV, or a composition comprising the rAAV for use in treating a neovascularization associated disease, an angiogenesis associated disease, or a VEGF-associated disease in a subject in need thereof, wherein the rAAV comprises an adeno-associated virus (AAV) capsid protein (e.g., AAV 2 variants or AAV2/3 hybrid variants) and an isolated nucleic acid comprising a transgene encoding an anti-VEGF agent (e.g., KH902).

In some embodiments, the delivery of the anti-VEGF agent results in at least 10%, at least 20%, at least 30%, at least 40%, at least 50%, at least 60%, at least 70%, at least 80%, at least 90%, or 100% inhibition of VEGF activity.

In some embodiments, the subject is a non-human mammal. In some embodiments, the non-human mammal is mouse, rat, cat, dog, sheep, rabbit, horse, cow, goat, pig, guinea pig, hamster, chicken, turkey, or a non-human primate. In some embodiments, the subject is a human.

In some embodiments, the subject has or is suspect of having an angiogenesis associated disease or a VEGF associated disease. In some embodiments, the VEGF associated disease is tumor, cancer, retinopathy, wet age-related macular degeneration (wAMD), macular edema, choroidal neovascularization, or corneal neovascularization.

In some embodiments, the administration is systemic administration, optionally wherein the administration is intravenous injection. In some embodiments, the administration is direct administration to ocular tissue, optionally wherein the direct administration is intravitreal injection, intraocular injection or topical administration.

In some embodiments, the administration results in delivery of the transgene to ocular tissue. In some embodiments, the ocular tissue comprises ocular neurons, retina, sclera, choroid, retina, vitreous body, macula, fovea, optic disc, lens, pupil, iris, aqueous fluid, cornea, conjunctiva ciliary body, or optic nerve.

In some embodiments, the administration results in inhibition of VEGF in the subject for at least 5 days, 10 days, 15 day, 20 days, 1 month, two months, or longer post administration.

In some aspects, the present disclosure provides a method of treating a corneal neovascularization (CNV) in a subject in need thereof, the method comprising administering to the subject an effective amount of the rAAV, or the pharmaceutical composition described herein. In some embodiments, the rAAV comprises an AAV8 capsid protein. In some aspects, the present disclosure provides a method of reducing corneal neovascularization (CNV) in a subject in need thereof (e.g., reducing CNV relative to an untreated subject, or in the subject prior to the administration), the method comprising administering to the subject an effective amount of the rAAV, or the pharmaceutical composition described herein. In some embodiments, the rAAV comprises an AAV8 capsid protein.

In some embodiments, the administration results in delivery of an anti-VEGF agent in corneal cells. In some embodiments, the administration results in delivery of an anti-VEGF agent in keratocytes of the cornea.

In some embodiments, the rAAV is administered once. In some embodiments, the administration results in expression of an anti-VEGF agent in corneal cells for longer than three months, six months, a year, or longer. In some embodiments, the administration results in inhibition of VEGF (e.g., VEGF expression or activity) in the subject for 1 month, two months, three months, six months, a year or longer post-administration. In some embodiments, the administration is intrastromal injection. In some embodiments, the subject is human. In some embodiments, the corneal neovascularization is acute corneal neovascularization or chronic corneal neovascularization.

### BRIEF DESCRIPTION OF DRAWINGS

FIGS. 1A-1C show the rAAV-CBA-KH902 vector and sequences. The expressed rAAV vector expresses a secreted KH902 (Conbercept) and is driven by the CMV enhancer and chicken β-actin promoter (CBA) cassette. A Kozak sequence was also designed 5′ of the start codon to enhance translation initiation. Map diagram (FIG. 1A) and read strand sequence (FIG. 1B, SEQ ID NO: 3) of the plasmid are shown. Sequences including and encompassed by the 5′-ITR and 3′-ITR are packaged into AAV virions (FIG. 1C).

FIG. 2 shows Western blot analysis of AAV-KH902-infected RPE-conditioned media. 15 μl of ARPE-19-(left) or hTERT-RPE1-(right) conditioned media for the designated conditions labeled above each lane were subjected to PAGE. Following semi-dry transfer, membranes were subjected to blotting with anti-VEGFR1 antibody (R&D Systems BAF321). 20 ng of KH902 drug (last lanes) was included as reference for each blot.

FIGS. 3A-3C show in vitro functional validation of AAV-KH902 vectors. Angiogenesis or the proliferative capacity of VEGF-stimulated (25 ng/mL) HUVECs, while in the presence of KH902; or conditioned media (diluted 1:10) of RPE cells infected with AAV2-KH902 or control GFP vector. Anti-VEGF activity was quantified by tube formation assays (FIGS. 3A and 3B) or by CCK-8 activity (FIG. 3C), respectively. *, p<0.01; **, p<0.001; ***, p<0.0001.

FIG. 4 shows that intravitreal rAAV2-KH902 injection prevents normal retinal vascular development. Neonatal mouse pups (P0-P3) were injected by intravitreal administration with rAAV2-KH902. Mice were raised in normoxic conditions (˜21% 02) and sacrificed at >P18. Retinas were mounted and stained with PECAM antibody (endothelial cells), or DAPI (DNA) and PNA (photoreceptors) and imaged from the ganglion cell side (top panels) or photoreceptor side (bottom panels).

FIGS. 5A-5C show intravitreal rAAV2-KH902 injection prevents retinal edemas in retinopathy of prematurity. Neonatal mice (P0-P3) were injected with rAAV2-KH902 and raised for approximately 4 days in normoxic conditions (˜21% 02) and then subjected to hyperoxic conditions (75% 02) for approximately 1 week. At P12-P18, mice were brought back to normoxic conditions for 6 days and sacrificed. (FIG. 5A) Retinas were mounted and stained with anti-Isolectin B4 (vascular stain) and anti-PECAM antibodies (endothelial cells). Each treatment group (n=6) were analyzed and tabulated for the occurrence of edema (FIG. 5B) and the number of cysts (FIG. 5C).

FIGS. 6A-6B show evaluation of rAAV2-KH902 in the oxygen-induced retinopathy mouse model. FIG. 6A shows bright field images of eyes injected with rAAV2-EGFP (left column) and rAAV2-KH902:rAAV2-EGFP at a 5:1 ratio mixture (right column) and imaged immediately after dissection. Eyes in the same row are from the same animal, therefore, rAAV2-Egfp injected eyes serve as controls for the extent of pathology induction within individual animals. FIG. 6B shows fluorescence imaging of eyes from a representative mouse were then flat-mounted and stained for Isolectin-B4. Areas of positive transduction are marked by EGFP expression. rAAV2-KH902 reduces normal vascular development and aneurysm nodules; i.e., strong EGFP expression has reduced retinal vasculature. Examples of aneurysm nodules are indicated in the bottom panel (arrows).

FIGS. 7A-7B show evaluation of rAAV8-KH902 in the oxygen-induced retinopathy mouse model. FIG. 7A shows bright field images of eyes injected with rAAV8-EGFP (left column) and rAAV8-KH902:rAAV8-EGFP at a 5:1 ratio mixture (right column) and imaged immediately after dissection. Eyes in the same row are from the same animal, therefore, rAAV8-Egfp injected eyes serve as controls for the extent of pathology induction within individual animals. FIG. 7B shows fluorescence imaging of eyes from a representative mouse were then flat-mounted and stained for Isolectin-B4. Areas of positive transduction are marked by EGFP expression. rAAV8-KH902 does not reduce normal vascular development and only modestly affects the formation of aneurysm nodules.

FIG. 8 shows percentage of rAAV treated eyes with pathologies. Mouse eyes in FIGS. 6A-6B and 7A-7B were scored for edemas or rescue. Experimental groups: rAAV2, n=10; rAAV8, n=10.

FIGS. 9A-9B show funduscopy of mouse eyes injected with rAAVs comprising AAV2 and AAV2/3-hybrid capsid variants and a nucleic acid encoding EGFP. Eight AAV2 variants (v224, v326, v358, v46, v56, v66, v67, and v81) and seven AAV2/3 hybrid variants (v439, v453, v513, v551, v556, v562, and v598) packaging CB6-EGFP were injected via intravitreal administration. Representative eyes were imaged at two weeks (FIG. 9A)) and four weeks (FIG. 9B) post-injection. Three capsids v56, v224, v326 (denoted by asterisks) were selected to package KH902. The number of eyes assessed (number of EGFP-positive eyes/number of all eyes) are noted at bottom right corner of each micrograph.

FIG. 10 shows treatment of laser damage-induced CNVs with vectored KH902 packaged by rAAV.v224. Mouse eyes were subjected to laser damage to induce choroidal neovascular (CNV) events. Five days after damage, intravitreal rAAV injections were performed. Longitudinal analysis of remaining CNVs following a control capsid encoding GFP and v224-KH902 was performed. rAAV v224-KH902 is capable of reducing the number to CNVs after laser damage to less than 80 percent 20 days post damage. Data represent mean±ME at 90% confidence.

FIG. 11 shows rAAV v224-KH902 does not cause lesions in the eye associated with immune cell infiltration into the vasculature in the eye.

FIG. 12 shows In vitro packaging yield assessment via crude-lysate PCR. Waterfall plots showing the relative packaging yields for AAV2 variants (top panel), AAV2/3 variants (middle panel), and AAV8 variants (bottom panel). The packaging yield values for each capsid are expressed as a percentage of yields conferred by their prototypic forms: AAV2, AAV3b, and AAV8, respectively. Capsid variants v56 showed 9.42 folds increase over AAV2; v224 showed 8.96 folds increase over AAV2, and v326 showed 9.79 folds increase over AAV2. The total number of capsids displayed are shown on the x-axes. AAV2/3 hybrid variants also showed 2 to 8 folds increase over AAV3b.

FIGS. 13A-13F show a comparison of corneal transduction between intrastromal and subconjunctival injections with rAAV8-eGFP. FIGS. 13A-13C show intrastromal injection of a mouse cornea. FIGS. 13D-13F show subconjunctival injection. FIGS. 13B and 13E show that eGFP signal was detected by live animal imaging at two weeks post-intrastromal injection with rAAV8 (1.6×10¹⁰ GCs in 4 μl per cornea). The dotted circle represents the edge of mouse cornea. FIGS. 13C, 13F show fluorescence microscopy of eGFP expression in representative cross-sections from FIGS. 13B and 13E, respectively. Arrows demarcate the site of injections. GCs: genome copies.

FIGS. 14A-14C show rAAV2- and rAAV8-mediated KH902 expression kinetics and cell tropism. FIG. 14A shows rAAV2- and rAAV8-mediated eGFP expression was detected at same intensity by live imaging microscopy at different time points, until three months (12 weeks) post-intrastromal injection. The dotted circle represents the edge of mouse cornea. FIG. 14B shows relative KH902 mRNA expression in rAAV8 and rAAV2 vector-treated mouse corneas (***p<0.001, ****p<0.0001). Data is shown as means±SEM, n=3. FIG. 14C shows histological analysis of cell specificity in cornea sections with rAAV2 and rAAV8. (i, ii) Anti-Vimentin staining of mouse corneas at two weeks after intrastromal injection with rAAV2- and rAAV8-mediated eGFP expression. The eGFP signal in the corneal stroma co-localized with the Vimentin labelled keratocytes. (iii, v) Anti-human IgG (H+L) labelling KH902 protein in the section of cornea intrastromally treated with rAAV8 or PBS, respectively. (iv) Higher magnification of the dashed-box regions in panel iii co-stained with anti-Vimentin co-staining (red). Magnifications a, b and d: 400×. Scale bar, 25 μm. Magnifications c: 200×. Scale bar, 500 Epi: epithelial layer; Endo: endothelial layer. The dose of each rAAV vectors for all the above experiments was 1.6×10¹⁰ GCs in 4 μl PBS per cornea.

FIGS. 15A-15D show measurement of central corneal thickness (CCT) and immune response towards rAAV2 and rAAV8 vectors. FIG. 15A shows OCT images of corneas pre-injection, immediately post-injection and at weeks 1, 2 and 12 post-injection of PBS, rAAV2-KH902 and rAAV8-eGFP/KH902 at the dose of 1.6×10¹⁰ GCs in 4 μl per cornea. FIG. 15B shows quantitative analysis of central corneal thickness measured from FIG. 15A images. FIG. 15C shows analysis of corneal immune responses to high-(1.6×10¹⁰ GCs/cornea) and low-dose (8×10⁸ GCs/cornea) rAAV2- or rAAV8-eGFP/KH902 with immunofluorescent staining for monocytes/macrophages (CD11b, F4/80, red). Magnifications: 200×. Scale bar, 50 μm. FIG. 15D shows calculated percentages of CD11b+ cells and F4/80+ cells in indicated groups from FIG. 15C data. ***, p<0.001; ****, p<0.0001. Data is shown as mean±SEM, n=5.

FIGS. 16A-16E show long-term inhibition of CoNV by rAAV8-KH902 via intrastromal delivery with the single dose in the alkali burn-induced CoNV model. FIG. 16A shows representative CoNV images of alkali-treated corneas injected with PBS, rAAV8-eGFP, Conbercept (10 mg/ml, 40), rAAV2-KH902, rAAV8-KH902 and rAAV8-KH902 combined with Conbercept (10 mg/ml, 40) at days 5 and 10, and weeks 2, 3, 4, 8, and 12. FIGS. 16B-16C show a histogram of CoNV area quantification in each condition from panel FIG. 16A data. †: significant difference from PBS group; *: significant difference from rAAV8-eGFP or KH902 groups; #: significant difference from rAAV2-KH902 group; †, * and, #p<0.05; ††, ** and ##, p<0.01; †††, *** and ###, p<0.001; ††††, **** and ####, p<0.0001. Data is shown as mean±SEM, n=5-7. FIG. 16D shows immunofluorescence analysis of mouse corneal flat mounts. The corneas in each condition of FIG. 16A harvested at 12 weeks after alkali burn were double stained, and the area covered by CD31⁺⁺⁺/LYVE-1—refers to blood vessels and CD31⁺/LYVE-1⁺⁺⁺ refers to lymph vessels (+++ indicates strong positivity; ++, medium positivity; and +, mild positivity). Magnification, 100×. Scale bar, 50 μm. FIG. 16E shows corneal angiogenesis and lymphangiogenesis analysis by measuring areas covered by CD31⁺⁺⁺ and LYVE-1⁺⁺⁺ staining respectively in each condition of FIG. 16D data. *: significant difference from PBS group; •: significant difference from rAAV8-eGFP group; #: significant difference from KH902 group; significant difference from rAAV2-KH902 group. ###, p<0.001; ffff, ****, ••••, #### and ffff, p<0.0001. Data is shown as mean±SEM, n=4. The dose of each rAAV vectors for the above experiments was 8×10⁸ GCs in 40 PBS or Conbercept solution per cornea.

FIGS. 17A-17F show rAAV8-delivered KH902 downregulated D114/Notch signaling and ERK activation in alkali burn-induced CoNV model. FIG. 17A shows immunofluorescence analysis of D114 expression in mouse corneal flat mounts co-stained with CD31 at two weeks post-alkali burn in PBS, rAAV8-eGFP and rAAV8-KH902 (8×10⁸ GCs/cornea) treated corneas. Magnifications: 200×. Scale bar, 100 μm. FIGS. 17B, 17C, 17D show Western blot with semi-quantitative analysis of D114 and NICD expression in mouse cornea at two weeks after alkali burn in each indicated treatment groups. FIGS. 17E, 17F show Western blot with semi-quantitative analysis of ERK activation. The results are presented as the ratio of phosphorylated ERK (pERK) to total ERK (pERK/ERK) in the indicated treatment groups at eight days after alkali burn. ***, p<0.001; ****, p<0.0001.

FIGS. 18A-18C show rAAV8-KH902 prevented progression of pre-existing CoNV in the alkali-burn injury model. FIG. 18A shows mouse corneas were performed with intrastromal injection of PBS, rAAV8-eGFP and rAAV8-KH902 (8×10⁸ GCs in 40 per cornea) at day 10 after alkali burn (baseline). Representative images of CoNV observed weekly over four weeks are shown. FIG. 18B shows quantification analysis of weekly CoNV area in each group shown in FIG. 18A. The asterisk indicates significant differences between the end time point of observation (week 4) and corresponding baseline (**p<0.01). Data is shown as mean±SEM, n=5. FIG. 18C shows weekly parallel comparison by quantified CoNV area between experimental groups of FIG. 18A for 4 weeks. (*p<0.05, **p<0.01, ***p<0.001). Data is shown as mean±SEM, n=5.

FIGS. 19A-19C show rAAV8-KH902 prevented progression of pre-existing neovascularization in the suture-induced CoNV model. FIG. 19A shows mouse corneas subjected to five-day suture placement (baseline) were treated with intrastromal injection of PBS, rAAV8-eGFP and rAAV8-KH902 (8×10⁸ GCs in 4 μl per cornea). Weekly representative images of CoNV are shown with a four-week follow-up. FIG. 19B shows quantification analysis of CoNV areas with comparing experimental groups from FIG. 19A for four weeks. (****p<0.0001). Data is shown as mean±SEM, n=4-7. FIG. 19C shows quantification analysis of weekly CoNV areas in each group from FIG. 19A data. The asterisks indicate significant differences between the end time point (week 4) and the baseline (****p<0.0001).

FIGS. 20A-20C show he corneal transduction of intrastromal injection and subconjunctival injection with rAAV2 vector-delivered eGFP. FIG. 20A shows a representative image of eGFP expression in the mouse cornea section detected by fluorescence microscopy at 2-week post intrastromal injection of rAAV2 vector (1.6×10¹⁰ GCs/cornea). FIGS. 20B, 20C show representative images of eGFP signal detected by live animal imaging at two weeks post-intrastromal or subconjunctival injection with rAAV2, respectively. The dotted circle represents the edge of mouse cornea under the imaging microscope.

FIGS. 21A-21B show histological analysis of KH902 expression mediated by rAAV2 vector in the cornea via intrastromal injection. FIG. 21A shows representative eyeball images of KH902 expression marked by anti-human IgG (H+L) antibody. Magnifications: 200×. Scale bar, 500 m. FIG. 21B shows higher magnification of the boxed regions in FIG. 21A with anti-Vimentin co-staining, indicating the expression of KH902 was mainly distributed in the corneal stroma layer. Magnifications: 400×. Scale bar, 100 m. The dose of rAAV2 vectors was 1.6×10¹⁰ GCs in 4 μl PBS per cornea.

FIGS. 22A-22C show representative data relating to Fundus Photography and Fluorescent angiography (FP and FFA) of non-human primates (NHPs) injected with KH902-encoding rAAVs. FIG. 22A shows representative data indicating injection of KH902-encoding rAAVs reduces Grade IV CNV lesions relative to control injections; Conbercept was used as a positive treatment control. FIG. 22B shows representative data indicating injection of KH902-encoding rAAVs reduces fluorescein leakage area relative to control injections; Conbercept was used as a positive treatment control. FIG. 22C shows representative data indicating the regression of light spots on the 29th day after administration as observed by FFA.

### DETAILED DESCRIPTION

Aspects of the disclosure relate to compositions and methods for expressing anti-VEGF agents (e.g., KH902) in a cell or subject. The disclosure is based, in part, on rAAVs comprising a capsid protein (e.g., an AAV2/3 capsid protein, AAV8 capsid protein, etc.) and an rAAV vector comprising a nucleic acid encoding an anti-VEGF agent flanked by adeno-associated virus (AAV) inverted terminal repeats (ITRs). In some embodiments, the nucleic acid comprises a promoter, such as a CMV promoter or a chicken beta-actin (CBA) promoter.

In some aspects, the rAAV disclosed herein includes an AAV capsid (e.g., AAV2 variant or AAV2/3 hybrid variant capsid protein) containing an isolated nucleic acid encoding a transgene expression cassette that comprises a nucleic acid sequence anti-vascular endothelial growth factor (e.g., an anti-VEGF) agent flanked by AAV inverted terminal repeats (ITRs). The disclosure is based, in part, on rAAVs engineered to express transgenes encoding anti-VEGF agent (e.g., a VEGF receptor fusion protein such as KH902) or variants thereof. In some embodiments, compositions described by the disclosure (e.g., rAAVs) are useful for treating diseases associated with the eye, for example corneal vascularization.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., ocular tissues). The AAV capsid is an important element in determining these tissue-specific targeting capabilities (e.g., tissue tropism). Thus, an rAAV having a capsid appropriate for the tissue being targeted can be selected.

The present disclosure, at least in part, relates to a recombinant adeno-associated virus (rAAV), comprising: (i) an AAV capsid protein, wherein the capsid protein is of AAV2 variant or AAV2/3 hybrid, and (ii) an isolated nucleic acid comprising a transgene encoding an anti-vascular endothelial growth factor (anti-VEGF) agent, the transgene being flanked by inverted terminal repeats (ITR)s. The present disclosure, at least in part, also relates to a recombinant adeno-associated virus (rAAV), comprising: (i) an AAV capsid protein, wherein the capsid protein is of AAV8 serotype, and (ii) an isolated nucleic acid comprising a transgene encoding an anti-vascular endothelial growth factor (anti-VEGF) agent, the transgene being flanked by inverted terminal repeats (ITR)s.

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for ocular tissues or muscle tissue. In some embodiments, an AAV capsid protein targets ocular cell types (e.g., photoreceptor cells, retinal cells, etc.).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP, and variants of any of the foregoing. In some embodiments, an AAV capsid protein is of a serotype derived from a non-human primate, for example AAVrh8 serotype. In some embodiments, the capsid protein is of AAV serotype 6 (e.g., AAV6 capsid protein), AAV serotype 8 (e.g., AAV8 capsid protein), AAV serotype 2 (e.g., AAV2 capsid protein), AAV serotype 5 (e.g., AAV5 capsid protein), or AAV serotype 9 (e.g., AAV9 capsid protein). In some embodiments, the AAV capsid is AAV1. In some embodiments, the AAV capsid is AAV2. In some embodiments, the AAV capsid protein with desired tissue tropism can be selected from AAV capsid proteins isolated from mammals (e.g., tissue from a subject). (See, for example, WO2010138263A2 and WO2018071831, the entire contents of which are incorporated herein by reference). In some embodiments, the AAV capsid is AAV8.

In some embodiments, an AAV capsid is a variant, or homolog of a known AAV capsid protein. In some embodiments, combinations of capsid protein variants and KH902 are confers benefits in rAAV based therapy (e.g., better packaging efficiency, effective inhibition of VEGF, or less toxicity associated with overexpression of KH902) than previously described capsids for delivering KH902. A capsid variant typically comprises at least one amino acid substitution, insertion, or deletion, relative to the wild-type capsid (or capsids) from which it is derived. In some embodiments, an AAV variant comprises between about 1 to about 100 amino acid (e.g., between 1-10 amino acids, between 1-20 amino acids, between 1-30 amino acids, between 20-50 amino acids, between 20-60 amino acids, between 50-80 amino acids, between 50-100 amino acids, between 60-100 amino acids, etc.) 1-substitution, insertion, or deletion compared with a known AAV capsid (e.g., AAV serotype 2, or AAV2/3 (e.g., AAV2/3 hybrid), etc.). In some embodiments, an AAV variant comprises more than 100 amino acid (e.g., between 100-200 amino acids, between 200-300 amino acids, between 100-500 amino acids, between 500-1000 amino acids or more) substitution, insertion, or deletion compared with a known AAV capsid (e.g., AAV serotype 2, or AAV2/3 (e.g., AAV2/3 hybrid), etc.). In some embodiments, an AAV variant may comprise between about 5 to about 50 amino acid (e.g., between 5-10 amino acids, between 5-20 amino acids, between 5-30 amino acids, between 5-40 amino acids, between 10-20 amino acids, between 10-30 amino acids, between 10-40 amino acids, between 10-50 amino acids, or between 30-50 amino acids) substitution, insertion or deletion compared with a known AAV serotype (e.g., AAV serotype 2, or AAV2/3 (e.g., AAV2/3 hybrid), etc.). In some embodiments, an AAV variant may comprise about 10 to about 30 amino acid (e.g., 10, 11, 12, 13, 14, 15, 16, 17, 18, 19, 20, 21, 22, 23, 24, 25, 26, 27, 28, 29, or 30) substitution, insertion or deletion compared with a known AAV serotype (e.g., AAV serotype 2, or AAV2/3 (e.g., AAV2/3 hybrid)). In some embodiments, an AAV variant may comprise 1, or 2, or 3, or 4, 5, or 6, or 7, or 8, or 9, or 10, or 11, or 12, or 13, or 14, or 15, or 16, or 17, or 18, or 19, or 20 amino acid substitution, insertion, or deletion compared with a known AAV serotype (e.g., AAV serotype 2, or AAV2/3 (e.g., AAV2/3 hybrid)). In some embodiments, an AAV variant comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of compared with a known AAV capsid (e.g., AAV serotype 2, or AAV2/3 (e.g., AAV2/3 hybrid), etc.).

In some embodiments, a capsid variant may be a chimeric capsid variant. A chimeric capsid variant sequence may comprise portions of two or more AAV capsid serotypes or variants thereof. In some embodiments, a chimeric capsid comprises portions of 2, 3, 4, 5, 6, 7, 8, 9, 10, or more different capsid protein serotypes. In some embodiments, chimeric capsid proteins have different properties, such as tissue tropism, etc., that the AAV capsid proteins from which they are derived. The fragments may be incorporated by any appropriate method, for example recombinant DNA cloning.

In some embodiments, the AAV variants described herein are variants of AAV2, AAV2/3 (e.g., AAV2/3 hybrid) or AAV8. AAV2 has been observed to efficiently transduce ocular tissue (e.g., photoreceptor cells and retinal pigment epithelium (RPE)), human central nervous system (CNS) tissue, kidney tissue, and other tissues. In some embodiments, an AAV capsid described herein is an AAV2 variant. Accordingly, in some embodiments, the AAV2 variants described herein may be useful for delivering gene therapy to ocular tissue (e.g., the retina). AAV3 has been observed to efficiently transduce cancerous human hepatocytes. In some embodiments, an AAV variant described herein is an AAV2/3 (e.g., AAV2/3 hybrid).

In some embodiments, a capsid variant (e.g., AAV2 variant, AAV 2/3 variant, or AAV8 variant) is any of the capsid variants as described in WO2018071831, the entire contents of which is incorporated herein by reference. In some embodiments, the AAV2 variant is v224, v326, v358, v46, v56, v66, v67, or v81. In some embodiments, the AAV2 variant is v224. In some embodiments, the AAV2 variant is v326. In some embodiments the AAV2 variant is v56. In some embodiments, the AAV2/3 hybrid is v439, v453, v513, v551, v556, v562, or v598. In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of a wild-type AAV2/3 amino acid sequence. In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of a wild-type AAV8 amino acid sequence. In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of a wild-type AAV2 amino acid sequence as set forth in SEQ ID NO: 11. An exemplary amino acid sequence of wild-type AAV2 is set forth in SEQ ID NO: 11:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v224 amino acid sequence as set forth in SEQ ID NO: 12. An exemplary amino acid sequence of v224 is set forth in SEQ ID NO: 12:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLARAVFQAKKRVLEPLGLVEEPVKTAPGKKRPVEHSP
AEPDSSSGTGKSGQQPARKRLNFGQTGDADSVPDPQPLGQPPAAPSGLGT
NTMASGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLRFSQAGASDIRDQSRNWLPGPCYRQQRVSKTAADNNNSDY
SWTGATKYHLNGRDSLVNPGTAMASHKDDEEKYFPQSGVLIFGKQDSEKT
NVDIERVMITDEEEIRTTNPVATEQYGSVSTNLQSGNTQAATSDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v326 amino acid sequence as set forth in SEQ ID NO: 13. An exemplary amino acid sequence of v326 is set forth in SEQ ID NO: 13:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMANHKDDEEKYFPQSGVLIFGKQGSNKT
NVDIEKVMITDEEEIRTTNPVATEQYGSVSTNLQSGNTQAATSDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v56 amino acid sequence as set forth in SEQ ID NO: 14. An exemplary amino acid sequence of v56 is set forth in SEQ ID NO: 14:

MAADGYLPDWLEDTLSEGIRQWWKLKPGPPPPKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHFP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQDSGKT
NVDIEKVMITDEEEIRTTNPVATEQYGSVSTNLQSGNTQAATTDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v358 amino acid sequence as set forth in SEQ ID NO: 15. An exemplary amino acid sequence of v358 is set forth in SEQ ID NO: 15:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VESDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQGSNKT
NVDIEKVMITDEEEIRTTNPVATEQYGSVSTNLQSGNTQAATSDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v46 amino acid sequence as set forth in SEQ ID NO: 16. An exemplary amino acid sequence of v46 is set forth in SEQ ID NO: 16:

MAADGYLPDWLEDTLSEGIRQWWKLKPGPPPPKPAERHQDDSRGLVLPGY
KYLGPFNGLDKGEPVSEADVAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQDSGKT
NVDIEKVMITDEEEIRTTNPVATEQYGSVSTNLQSGNTQAATTDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v66 amino acid sequence as set forth in SEQ ID NO: 17. An exemplary amino acid sequence of v66 is set forth in SEQ ID NO: 17:

MAADGYLPDWLEDTLSEGIRQWWKLKPGPPPPKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQDSGKT
NVDIEKVMITDEEEIRTTNPVATEQYGSVSTNLQSGNTQAATTDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v67 amino acid sequence as set forth in SEQ ID NO: 18. An exemplary amino acid sequence of v67 is set forth in SEQ ID NO: 18:

MAADGYLPDWLEDTLSEGIRQWWKLKPGPPPPKPAERHQDDSRGLVLPGY
KYLGPFNGLDKGEPVNEADVAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQDSGKT
NVDIEKVMITDEEEIRTTNPVATEQYGSVSTNLQSGNTQAATTDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2 variant v81 amino acid sequence as set forth in SEQ ID NO: 19. An exemplary amino acid sequence of v81 is set forth in SEQ ID NO: 19:

MAADGYLPDWLEDTLSEGIRQWWKLKPGPPPLKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPSGLGT
NTMAAGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSKTNA
PSGTTTMSRLQFSQAGASDIRDQSRNWLPGPCYRQQRVSKTAADNNNSDY
SWTGATKYHLNGRDSLVNPGPAMASHKDDEEKYFPQSGVLIFGKQDSGKT
NVDIEKVMITDEEEIRTTNPVATEQYGSVSTNLQSGNTQAATTDVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v439 amino acid sequence as set forth in SEQ ID NO: 20. An exemplary amino acid sequence of v439 is set forth in SEQ ID NO: 20:

MAADGYLPDWLEDTLSEGIRQWWKLKPGPPPPRPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPTSLGS
TTMATGSGAPMAGNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNRTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLEHVMITDEEEIRTTNPVATEQYGNVSNNLQNSNTGPTTENVNHQGA
LPGMVWQDRDVYLQGPIWAKIPHTDGHFHPSPLMGGFGLKHPPPQIMIKN
TPVPANPPTNFSAAKFASFITQYSTGQVSVEIEWELQKENSKRWNPEIQY
TSNYNKSVNVDFTVDANGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v453 amino acid sequence as set forth in SEQ ID NO: 21. An exemplary amino acid sequence of v453 is set forth in SEQ ID NO: 21:

MAADGYLPDWLEDTLSEGIRQWWKLKPGPPPPRPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPPGQPPAAPTSLGS
TTMATGSGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNRTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLEHVMITDEEEIRTTNPVATEQYGNVSNNLQNSNTGPTTENVNHQGA
LPGMVWQDRDVYLQGPIWAKIPHTDGHFHPSPLMGGFGLKHPPPQIMIKN
TPVPANPPTNFSAAKFASFITQYSTGQVSVEIEWELQKENSKRWNPEIQY
TSNYNKSVNVDFTVDANGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v513 amino acid sequence as set forth in SEQ ID NO: 22. An exemplary amino acid sequence of v513 is set forth in SEQ ID NO: 22:

MAADGYLPDWLEDTLSEGIRQWWKLKPGPPPPKPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPTSLGS
TTMATGSGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNRTQS
NSGTLQQSRLLFSQAGPTSMSLQAKNWLPGPCYRQQRLSKQANDNNSNFP
WTAATKYHLNGRDSLVNPGPAMASHKDDEEKFFPMHGTLIFGKQGTNAND
ADLEHVMITDEEEIRTTNPVATEQYGNVSNNLQNSNTGPTTENVNHQGAL
PGMVWQDRDVYLQGPIWAKIPHTDGHFHPSPLMGGFGLKHPPPQIMIKNT
PVPANPPTNFSAAKFASFITQYSTGQVSVEIEWELQKENSKRWNPEIQYT
SNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v551 amino acid sequence as set forth in SEQ ID NO: 23. An exemplary amino acid sequence of v551 is set forth in SEQ ID NO: 23:

MAADGYLPDWLEDTLSEGIRQWWKLKPGPPPPRPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPTSLGS
TTMATGSGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVSFHSSYAHSQSLDRLMNPLIDQYLYYLNRTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLEHVMITDEEEIRTTNPVATEQYGNVSNNLQNSNTGPTTENVNHQGA
LPGMVWQDRDVYLQGPIWAKIPHTDGHFHPSPLMGGFGLKHPPPQIMIKN
TPVPANPPTNFSAAKFASFITQYSTGQVSVEIEWELQKENSKRWNPEIQY
TSNYNKSVNVDFTVDANGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v556 amino acid sequence as set forth in SEQ ID NO: 24. An exemplary amino acid sequence of v556 is set forth in SEQ ID NO: 24:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKRRVLEPLGLVEEPVKTAPGKKRPVEHSP
VEPDSSSGTGKAGQQPARKRLNFGQTGDADSVPDPQPLGQPPAAPTSLGS
TTMATGSGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNKTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLDNVMITDEEEIRTTNPVATEQYGTVSNNLQNSNTGPTTGTVNHQGA
LPGMVWQDRDVYLQGPIWAKIPHTDGHFHPSPLMGGFGLKHPPPQIMIKN
TPVPANPPTNFSSAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v562 amino acid sequence as set forth in SEQ ID NO: 25. An exemplary amino acid sequence of v562 is set forth in SEQ ID NO: 25:

MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPTSLGS
TTMATGSGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTGTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNKTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLDNVMITDEEEIRTTNPVATEQYGTVSNNLQNSNTGPTTGTVNHQGA
LPGMVWQDRDVYLQGPIWAKIPHTDGHFHPSPLMGGFGLKHPPPQIMIKN
TPVPANPPTNFSSAKFASFITQYSTGQVSVEIEWELQKENSKRWNPEIQY
TSNYNKSVNVDFTVDTNGVYSEPRPIGTRYLTRNL

In some embodiments, the capsid protein comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of an AAV2/3 hybrid variant v598 amino acid sequence as set forth in SEQ ID NO: 26. An exemplary amino acid sequence of v598 is set forth in SEQ ID NO: 26:

MAADGYLPDWLEDTLSEGIRQWWKLKPGPPPPRPAERHQDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
AEPDSSSGTGKAGQQPARKRLNFGQTGDADSVPDPQPLGQPPAAPTSLGS
TTMATGSGAPMADNNEGADGVGNSSGNWHCDSQWLGDRVITTSTRTWALP
TYNNHLYKQISSQSGASNDSHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLNRTQS
NSGTLQQSRLLFSQAGPTSMSLQAKNWLPGPCYRQQRLSKQANDNNNSNF
PWTAATKYHLNGRDSLVNPGPAMASHKDDEEKFFPMHGTLIFGKQGTNAN
DADLEHVMITDEEEIRTTNPVATEQYGNVSNNLQNSNTGPTTENVNHQGA
LPGMVWQDRDVYLQGPIWAKIPHTDGHFHPSPLMGGFGLKHPPPQIMIKN
TPVPANPPTNFSAAKFASFITQYSTGQVSVEIEWELQKENSKRWNPEIQY
TSNYNKSVNVDFTVDANGVYSEPRPIGTRYLTRNL

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

The components to be cultured in the host cell to package an rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, an AAV capsid protein described herein confers better packaging efficiency than a reference AAV capsid protein. AAV packaging efficiency, as used herein, refers to the percentage of AAV virions with encapsidated intact genomes in a batch of produced AAV virions. Packaging efficiency can be determined by any known methods suitable for determining packaging efficiency (e.g., by crude lysate PCR or by infecting cells and evaluate transgene expression as described in Zhou et al., In Vitro Packaging of Adeno-Associated Virus DNA, J Virol. 1998 April; 72(4): 3241-3247). In some embodiments, a better packaging efficiency refers to at least more than 10%, at least more than 20%, at least more than 30%, at least more than 40%, at least more than 50%, at least more than 60%, at least more than 70%, at least more than 80%, at least more than 90%, at least more than 100%, at least more than 150%, at least more than 200%, at least more than 250%, at least more than 300%, at least more than 350%, at least more than 400%, at least more than 450%, at least more than 500% or more AAV virions with encapsidated intact genomes than a reference capsid protein. In some embodiments, a better packaging efficiency refers to at least 1-fold, at least 2-fold at least 3-fold, at least 4-fold, at least 5-fold, at least 6-fold, at least 7-fold, at least 8-fold, at least 9-fold, at least 10-fold, at least 10 to 50-fold (e.g., 10-fold, 20-fold, 30-fold, 40-fold, or 50-fold), at least 50 to 100-fold (e.g., 50-fold, 60-fold, 70-fold, 80-fold, 90-fold or 100-fold) or more of AAV virions with encapsidated intact genomes than a reference capsid protein. In some embodiments, the reference capsid protein is the prototypic capsid protein from with the capsid variants derive from (e.g., AAV2 or AAV3b capsid proteins). In some embodiments, the capsid variants described herein confers better packaging efficiency as compared to the prototypic capsid they derive from. In some embodiments, the AAV2 variants described herein (e.g., v224, v56, or v326) has a packaging efficiency at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold or higher as compared to AAV2. In some embodiments, the AAV2/3 hybrid capsid proteins described herein (e.g., v439, v453, v513, v551, v556, v562, or v598) has a packaging efficiency at least 1-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold or higher as compared to AAV3b.

In some embodiments, the disclosure relates to a host cell containing a nucleic acid that comprises a coding sequence encoding a transgene (e.g., KH902). A “host cell” refers to any cell that harbors, or is capable of harboring, a substance of interest. Often a host cell is a mammalian cell. In some embodiments, a host cell is a photoreceptor cell, retinal pigment epithelial cell, keratinocyte, corneal cell, and/or a tumor cell. A host cell may be used as a recipient of an AAV helper construct, an AAV vector, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron, a photoreceptor cell, a pigmented retinal epithelial cell, or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

Isolated Nucleic Acids Encoding the Trans Gene

The disclosure relates, in some aspects, to isolated nucleic acids encoding an anti-vascular endothelial growth factor (anti-VEGF) protein. Vascular endothelial growth factor (VEGF), originally known as vascular permeability factor (VPF), is a signal protein produced by cells that stimulates the formation of blood vessels. VEGF is a sub-family of growth factors, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). VEGF's normal function is to create new blood vessels during embryonic development, new blood vessels after injury, muscle following exercise, and new vessels (collateral circulation) to bypass blocked vessels. However, aberrant VEGF activity/signaling contributes to various diseases, such as vascular diseases.

Anti-vascular endothelial growth factor therapy, also known as anti-VEGF therapy or anti-VEGF medication, is the use of medications that block vascular endothelial growth factor activity. Non-limiting examples of anti-VEGF agent include VEGF receptor fusion protein (e.g., KH902), monoclonal antibodies such as bevacizumab, antibody derivatives such as ranibizumab (Lucentis), or orally-available small molecules that inhibit the tyrosine kinases stimulated by VEGF (e.g., lapatinib, sunitinib, sorafenib, axitinib, and pazopanib).

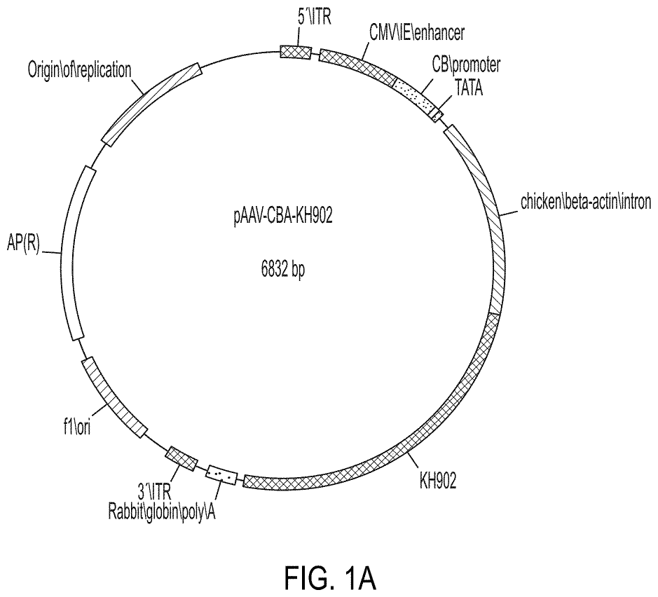

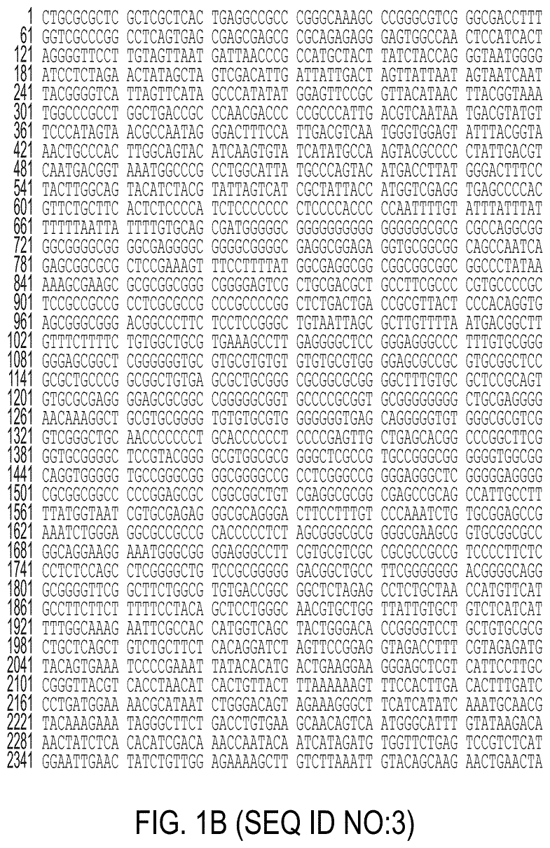

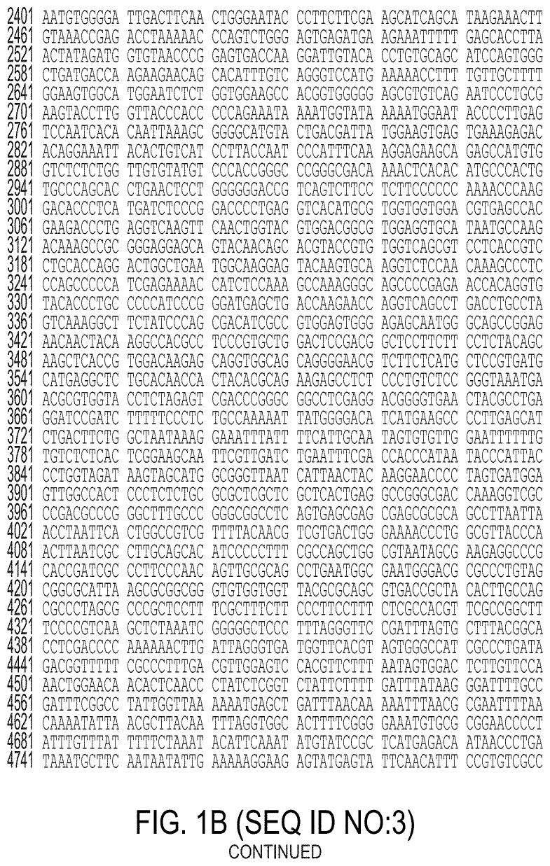

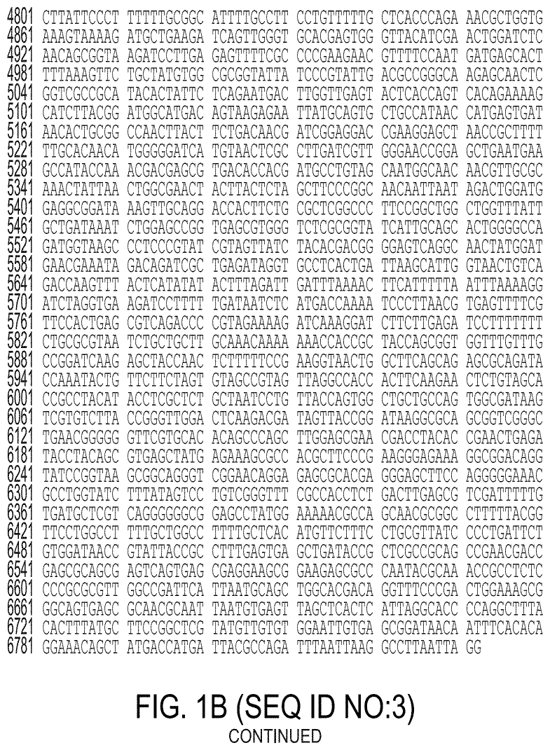

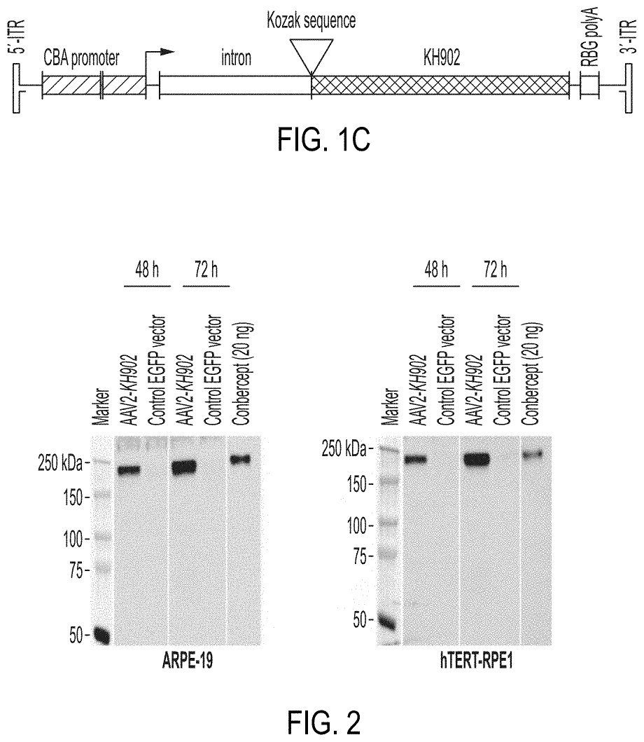

In some embodiments, isolated nucleic acids described herein comprises a transgene encoding an anti-VEGF agent. In some embodiments, the anti-VEGF agent targets (e.g., specifically binds to) a human VEGF receptor. VEGF receptors are receptors for vascular endothelial growth factor (VEGF). There are three main subtypes of VEGF receptor, numbered 1, 2 and 3. VEGFR-1, VEGFR-2, and VEGFR-3 belong to the receptor tyrosine kinase family (FIG. 1A). VEGFR-1 and -2 are primarily involved in angiogenesis, whereas VEGFR-3 are involved in hematopoiesis and lymphangiogenesis. The VEGFRs contain an approximately 750-amino-acid-residue extracellular domain, which is organized into seven immunoglobulin-like folds. Adjacent to the extracellular domain is a single transmembrane region, followed by a juxtamembrane domain, a split tyrosine-kinase domain that is interrupted by a 70-amino-acid kinase insert, and a C-terminal tail. VEGF receptor activation requires dimerization. Guided by the binding properties of the ligands, VEGFRs form both homodimers and heterodimers. Dimerization of VEGFR is accompanied by activation of receptor kinase activity, leading to autophosphorylation. Signal transduction is propagated when activated VEGF receptors phosphorylate SH2 domain-containing protein substrates. Vascular endothelial growth factor (VEGF) is an important signaling protein involved in many biological pathways (e.g., vasculogenesis and angiogenesis). The VEGF receptors have an extracellular portion consisting of 7 immunoglobulin-like domains (e.g., extracellular domain 1-7), a single transmembrane spanning region and an intracellular portion containing a split tyrosine-kinase domain. In some embodiments, human VEGF receptor 1 comprises an amino acid sequence as set forth in NCBI Accession No. NP_001153392.1, NCBI Accession No. NP_001153502.1, NCBI Accession No. NP_001153503.1, or NCBI Accession No. NP_002010.2. In some embodiments, human VEGF receptor 2 comprises an amino acid sequence as set forth in NCBI Accession No. NP_002244.1. In some embodiments, human VEGF receptor 3 comprises an amino acid sequence as set forth in NCBI Accession No. NP_002011.2, NCBI Accession No. NP_001341918.1. or NCBI Accession No. NP_891555.2. Vascular endothelial growth factor (VEGF) is an important signaling protein involved in many biological pathways (e.g., vasculogenesis and angiogenesis). The VEGF receptors have an extracellular portion consisting of 7 immunoglobulin-like domains (e.g., extracellular domain 1-7), a single transmembrane spanning region and an intracellular portion containing a split tyrosine-kinase domain. In some embodiments, an anti-VEGF agent targets (e.g., specifically binds to) a placental-derived growth factor (PlGF).

In some embodiments, the anti-VEGF agent is a human VEGF decoy receptor, or a portion thereof. A “decoy receptor” refers to a receptor that is able to recognize and bind a ligand (e.g., VEGF), but is not structurally able to signal or activate the cognate receptor complex of the ligand. The VEGF decoy receptor acts as an inhibitor, binding a ligand and keeping it from binding to its regular receptor. In some embodiments, the VEGF decoy receptor comprises one or more extracellular domains of the VEGF receptor 1 and/or VEGF receptor 2. In some embodiments, the anti-VEGF agent is a human VEGF decoy receptor fusion protein. In some embodiments, the human VEGF decoy receptor fusion protein comprises more than one extra cellular domains selected from VEGF receptor 1 and/or VEGF receptor 2 fused together. In some embodiments, the human VEGF decoy receptor fusion protein comprises a first portion including a VEGF receptor 1 fused to a VEGF receptor 2, which is further fused to second portion comprising a different protein (e.g., Fc portion of an immunoglobulin). VEGF decoy receptors and VEGF decoy receptor fusion proteins have been previously described, see. e.g., WO2007112675, and EP1767546B1, the entire contents of which are incorporated herein by reference.

In some embodiments, the human VEGF decoy receptor comprises an extracellular domain of a protein that binds VEGF. In some embodiments, the human VEGF decoy receptor comprises an extracellular domain of human VEGF receptor 1. In some embodiments, the human VEGF decoy receptor comprises extracellular domain 2 of human VEGF receptor 1. In some embodiments, the human VEGF decoy receptor comprises an extracellular domain of human VEGF receptor 2. In some embodiments, the human VEGF decoy receptor comprises extracellular domains 3 and 4 of human VEGF receptor 2.

In some embodiments, the human VEGF decoy receptor is a human VEGF receptor fusion protein. In some embodiments, the VEGF receptor fusion protein comprises an extracellular domain selected from VEGF receptor 1 or VEGF receptor 2, and one or more second extracellular domain selected from VEGF receptor 1 or VEGF receptor 2. In some embodiments, the VEGF receptor fusion protein comprises extracellular domain 2 of VEGF receptor 1, and extracellular domain 3 of VEGF receptor 2. In some embodiments, the VEGF receptor fusion protein comprises extracellular domain 2 of VEGF receptor 1, and extracellular domains 3 and 4 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 1. In some embodiments, the VEGF receptor fusion protein comprises extracellular domain 1 of VEGF receptor 2, fused to extracellular domain 2 of VEGF receptor 1, and further fused to extracellular domain 3 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 2, and further fused to extracellular domain 5 of VEGF receptor 2. In some embodiments, the VEGF receptor fusion protein comprises extracellular domain 2 of VEGF receptor 1, fused to extracellular domain 3 of VEGF receptor 2, and further fused to extracellular domain 4 of VEGF receptor 2, and further fused to extracellular domain 5 of VEGF receptor 1. In some embodiments, the fused extracellular domains of a VEGF decoy receptor are connected to one another by a linker. In some embodiments, the fused extracellular domains of a VEGF decoy receptor are connected to one another directly.

In addition, any of the VEGF receptor fusion proteins described herein may be fused to another protein. In some embodiments, the VEGF receptor fusion protein comprises a portion that is VEGF receptor (e.g., any of the VEGF decoy receptor or VEGF decoy receptor fusion protein described herein) fused to another protein to provide dimerization or multimerization properties. Non-limiting examples of the protein to provide dimerization or multimerization properties for the fusion protein is the Fc portion of an immunoglobulin. In some embodiments, the VEGF receptor fusion protein comprises a portion that is VEGF receptor (e.g., any of the VEGF decoy receptor or VEGF decoy receptor fusion protein described herein) is fused to an Fc portion of an immunoglobulin. In some embodiments, the VEGF receptor fusion protein (e.g., a VEGF decoy receptor or a VEGF decoy receptor fusion protein described herein) is fused to the other portion (e.g., an Fc domain) directly. In some embodiments, VEGF receptor fusion protein (e.g., a VEGF receptor decoy) is fused to the other portion via a linker.

Suitable linkers are known in the art. (See, e.g., Chen et al., Fusion protein linkers: property, design and functionality, Adv Drug Deliv Rev. 2013 October; 65(10):1357-69). In some embodiments, the VEGF receptor fusion protein is further fused to an Fc portion of an immunoglobulin. In some embodiments, the VEGF receptor fusion protein is KH902. KH902, also known as Conbercept (e.g., US20100272719A1, the entire contents which are incorporated herein by reference) is a decoy receptor protein constructed by fusing vascular endothelial growth factor (VEGF) receptor 1 and VEGF receptor 2 extracellular domains with the Fc region of human immunoglobulin. The size of KH902 is about 142 kD. Conbercept-mediated blockage of VEGF and placental growth factor (PIGF), which can induce neovascularization, has been proven to effectively treat wet age-related macular degeneration (wAMD) in clinical trials, including phase 3 trials, see. e.g., Liu et al., AJO, Aug. 17, 2019, the entire contents of which are incorporated herein by reference.

In some embodiments, the anti-VEGF agent comprises an amino acid sequence at least at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence as set forth in SEQ ID NO: 5. An exemplary amino acid sequence for KH902 is set forth in SEQ ID NO: 5.

(SEQ ID NO: 5)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
IPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSFFLYSKLTVDKSRWQQGNVFSCSVMHEALHNHYTQKSLSLSP
GK

In some embodiments, the anti-VEGF agent comprises a portion of SEQ ID NO: 5. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 as set forth in SEQ ID NO: 6. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 3 and 4 of VEGF receptor 2 as set forth in SEQ ID NO: 7. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 fused to extracellular domain 2 of VEGF receptor 1 as set forth in SEQ ID NO: 8. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 2 of VEGF receptor 1 fused to an Fc portion of an immunoglobulin as set forth in SEQ ID NO:9. In some embodiments, the anti-VEGF agent comprises an amino acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the amino acid sequence of extracellular domain 3 and 4 of VEGF receptor 2 fused to an Fc portion of an immunoglobulin as set forth in SEQ ID NO: 10. An exemplary amino acid sequence of extracellular domain 2 of VEGF receptor 1 is set forth in SEQ ID NO: 6:

(SEQ ID NO: 6)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDV

An exemplary amino acid sequence of extracellular domain 3 and 4 of VEGF receptor 2 is set forth in SEQ ID NO: 7:

(SEQ ID NO: 7)
VLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHE
KPFVAFGSGMESLVEATVGERVRIPAKYLGYPPPEIKWYKNGIPLESNHT
IKAGHVLTIMEVSERDTGNYTVILTNPISKEKQSHVVSLVVYVPP

An exemplary amino acid sequence of extracellular domain 2 of VEGF receptor 1 fused to extracellular domain 3 and 4 of VEGF receptor 2 is set forth in SEQ ID NO: 8:

(SEQ ID NO: 8)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
IPAKYLGYPPPEIKWYKNGIPLESNHTIKAGHVLTIMEVSERDTGNYTVI
LTNPISKEKQSHVVSLVVYVPP

An exemplary amino acid sequence of extracellular domain 2 of VEGF receptor 1 fused to Fc portion is set forth in SEQ ID NO: 9

(SEQ ID NO: 9)
MVSYWDTGVLLCALLSCLLLTGSSSGGRPEVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVGPGDKTHTCPLCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENN
YKATPPVLDSDGSFFLYSKLTVDKSRWQQGNVFSCSVMHEALHNHYTQKS
LSLSPGK

An exemplary amino acid sequence of extracellular domain 3 and 4 of VEGF receptor 2 fused to Fc portion is set forth in SEQ ID NO: 10:

(SEQ ID NO: 10)
VLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHE
KPFVAFGSGMESLVEATVGERVRIPAKYLGYPPPEIKWYKNGIPLESNHT
IKAGHVLTIMEVSERDTGNYTVILTNPISKEKQSHVVSLVVYVPPGPGDK
THTCPLCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKATPPVLDSDGSFFLYSKLTVDKSRWQQGNV
FSCSVMHEALHNHYTQKSLSLSPGK

In some embodiments, the isolated nucleic acid comprises a nucleic acid sequence at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 1. An exemplary coding sequence for KH902 is set forth in SEQ ID NO: 1.

(SEQ ID NO: 1)
ATGGTCAGCTACTGGGACACCGGGGTCCTGCTGTGCGCGCTGCTCAGCTG
TCTGCTTCTCACAGGATCTAGTTCCGGAGGTAGACCTTTCGTAGAGATGT
ACAGTGAAATCCCCGAAATTATACACATGACTGAAGGAAGGGAGCTCGTC
ATTCCCTGCCGGGTTACGTCACCTAACATCACTGTTACTTTAAAAAAGTT
TCCACTTGACACTTTGATCCCTGATGGAAAACGCATAATCTGGGACAGTA
GAAAGGGCTTCATCATATCAAATGCAACGTACAAAGAAATAGGGCTTCTG
ACCTGTGAAGCAACAGTCAATGGGCATTTGTATAAGACAAACTATCTCAC
ACATCGACAAACCAATACAATCATAGATGTGGTTCTGAGTCCGTCTCATG
GAATTGAACTATCTGTTGGAGAAAAGCTTGTCTTAAATTGTACAGCAAGA
ACTGAACTAAATGTGGGGATTGACTTCAACTGGGAATACCCTTCTTCGAA
GCATCAGCATAAGAAACTTGTAAACCGAGACCTAAAAACCCAGTCTGGGA
GTGAGATGAAGAAATTTTTGAGCACCTTAACTATAGATGGTGTAACCCGG
AGTGACCAAGGATTGTACACCTGTGCAGCATCCAGTGGGCTGATGACCAA
GAAGAACAGCACATTTGTCAGGGTCCATGAAAAACCTTTTGTTGCTTTTG
GAAGTGGCATGGAATCTCTGGTGGAAGCCACGGTGGGGGAGCGTGTCAGA
ATCCCTGCGAAGTACCTTGGTTACCCACCCCCAGAAATAAAATGGTATAA
AAATGGAATACCCCTTGAGTCCAATCACACAATTAAAGCGGGGCATGTAC
TGACGATTATGGAAGTGAGTGAAAGAGACACAGGAAATTACACTGTCATC
CTTACCAATCCCATTTCAAAGGAGAAGCAGAGCCATGTGGTCTCTCTGGT
TGTGTATGTCCCACCGGGCCCGGGCGACAAAACTCACACATGCCCACTGT
GCCCAGCACCTGAACTCCTGGGGGGACCGTCAGTCTTCCTCTTCCCCCCA
AAACCCAAGGACACCCTCATGATCTCCCGGACCCCTGAGGTCACATGCGT
GGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAG
TACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAA
CCACAGGTGTACACCCTGCCCCCATCCCGGGATGAGCTGACCAAGAACCA
GGTCAGCCTGACCTGCCTAGTCAAAGGCTTCTATCCCAGCGACATCGCCG
TGGAGTGGGAGAGCAATGGGCAGCCGGAGAACAACTACAAGGCCACGCCT
CCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAATGA

Any of the anti-VEGF agent described herein and or a combination thereof can be expressed by an isolated nucleic acid herein. In some embodiments, the isolated nucleic acid comprises a first region encoding the extracellular domain 2 of VEGF receptor 1 and a second region encoding the extracellular domain 3 and 4 of VEGF receptor 2. In some embodiments, the isolated nucleic acid comprises a first region encoding the extracellular domain 2 of VEGF receptor 1 fused to an Fc portion of an immunoglobulin and a second region encoding the extracellular domain 3 and 4 of VEGF receptor 2 fused to an Fc portion of an immunoglobulin. In some embodiments, the first region may be positioned at any suitable location. The first region maybe positioned upstream of the second region. For example, the first region may be positioned between the first codon of the second region and 2000 nucleotides upstream of the first codon. The first region may be positioned between the first codon of the second region and 1000 nucleotides upstream of the first codon. The first region may be positioned between the first codon of the second region and 500 nucleotides upstream of the first codon. The first region may be positioned between the first codon of the second region and 250 nucleotides upstream of the first codon. The first region may be positioned between the first codon of the second region and 150 nucleotides upstream of the first codon. In other embodiments, the first region may be positioned downstream of the second region. The first region may be between the last codon of the second region and a position 2000 nucleotides downstream of the last codon. The first region may be between the last codon of the second region and a position 1000 nucleotides downstream of the last codon. The first region may be between the last codon of second region and a position 500 nucleotides downstream of the last codon. The first region may be between the last codon of the second region and a position 250 nucleotides downstream of the last codon. The first region may be between the last codon of the second region and a position 150 nucleotides downstream of the last codon.

In some embodiments, the nucleic acid may also comprise a third region. In some embodiments, the isolated nucleic acid comprises a first region encoding the extracellular domain 2 of VEGF receptor 1, a second region encoding the extracellular domain 3 and 4 of VEGF receptor 2 and a third region encoding the extracellular domain 2 of VEGF receptor 1 fused to the extracellular domain 3 and 4 of VEGF receptor 2. In some embodiments, the isolated nucleic acid comprises a first region encoding the extracellular domain 2 of VEGF receptor 1 fused to an Fc portion of an immunoglobulin, a second region encoding the extracellular domain 3 and 4 of VEGF receptor 2 fused to an Fc portion of an immunoglobulin and a third region encoding the extracellular domain 2 of VEGF receptor 1 fused to the extracellular domain 3 and 4 of VEGF receptor 2, and further fused to an Fc portion of an immunoglobulin. In some embodiments, the third region of positioned upstream of the first codon of the first region. In some embodiments, the third region is positioned between the last codon of the first region and the first codon of the second region. In some embodiments, the third region is positioned downstream of the last codon of the second region.

In some embodiments, the various regions of an isolated nucleic acid disclosed herein are expression cassettes for expressing the anti-VEGF agent or a combination of anti-VEGF agents described herein. In some embodiments, a multicistronic expression construct comprises two or more expression cassettes encoding one or more anti-VEGF agents or a combination of anti-VEGF agents described herein.

In some embodiments, multicistronic expression constructs are comprise expression cassettes that are positioned in different ways. For example, in some embodiments, a multicistronic expression construct is provided in which a first expression cassette (e.g., an expression cassette encoding a first anti-VEGF agent, or portion thereof) is positioned adjacent to a second expression cassette (e.g., an expression cassette encoding a second anti-VEGF agent, or a portion thereof). In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

In different embodiments, multicistronic expression constructs are provided in which the expression cassettes are oriented in different ways. For example, in some embodiments, a multicistronic expression construct is provided in which a first expression cassette is in the same orientation as a second expression cassette. In some embodiments, a multicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1-promoter2/encoding sequence 2-3′,
>>>>>>>>>>>>>>>>>>>>>>>>>>>>>>>>>>>>>>>>>>>>>>
   the expression cassettes are in the same orientation, the arrows indicate the direction of transcription of each of the cassettes. For another example, if a given nucleic acid construct comprises a sense strand comprising two expression cassettes in the configuration
5′-promoter 1/encoding sequence 1-encoding sequence 2/promoter 2-3′,
>>>>>>>>>>>>>>>>>>>>>>><<<<<<<<<<<<<<<<<<<<<
   the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR, or in opposite orientation. AAV ITRs are directional. For example, the 3′ITR would be in the same orientation as the promoter1/encoding sequence 1 expression cassette of the examples above, but in opposite orientation to the 5′ITR, if both ITRs and the expression cassette would be on the same nucleic acid strand.

A large body of evidence suggests that multicistronic expression constructs often do not achieve optimal expression levels as compared to expression systems containing only one cistron. One of the suggested causes of sub-par expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulating properties of two genetic elements in an adenoviral vector containing two different expression cassettes. Hum Gene Ther. 2004 October; 15(10):995-1002; both references incorporated herein by reference for disclosure of promoter interference phenomenon). Various strategies have been suggested to overcome the problem of promoter interference, for example, by producing multicistronic expression constructs comprising only one promoter driving transcription of multiple encoding nucleic acid sequences separated by internal ribosomal entry sites, or by separating cistrons comprising their own promoter with transcriptional insulator elements. All suggested strategies to overcome promoter interference are burdened with their own set of problems, though. For example, single-promoter driven expression of multiple cistrons usually results in uneven expression levels of the cistrons. Further some promoters cannot efficiently be isolated and isolation elements are not compatible with some gene transfer vectors, for example, some retroviral vectors.

In some embodiments of this invention, a multicistronic expression construct is provided that allows efficient expression of a first encoding nucleic acid sequence driven by a first promoter and of a second encoding nucleic acid sequence driven by a second promoter without the use of transcriptional insulator elements. Various configurations of such multicistronic expression constructs are provided herein, for example, expression constructs harboring a first expression cassette comprising an intron and a second expression cassette positioned within the intron, in either the same or opposite orientation as the first cassette. Other configurations are described in more detail elsewhere herein.

In some embodiments, multicistronic expression constructs are provided allowing for efficient expression of two or more encoding nucleic acid sequences. In some embodiments, the multicistronic expression construct comprises two expression cassettes. In some embodiments, a first expression cassette of a multicistronic expression construct as provided herein comprises a first RNA polymerase II promoter and a second expression cassette comprises a second RNA polymerase II promoter. In some embodiments, a first expression cassette of a multicistronic expression construct as provided herein comprises an RNA polymerase II promoter and a second expression cassette comprises an RNA polymerase III promoter.

In some embodiments, the multicistronic expression construct provided is a recombinant AAV (rAAV) construct.

In some embodiments, the isolated nucleic acid described herein comprises a codon optimized nucleic acid sequence of an anti-VEGF agent (e.g., KH902). Codon optimization of the nucleic acid coding sequence for optimized expression in target cells (e.g., mammalian cells) can be achieved by methods known in the art.

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, isolated nucleic acid and rAAVs described herein comprise one or more of the following structural features (e.g., control or regulatory sequences): a long Chicken Beta Actin (CBA) promoter, an extended CBA intron, a Kozak sequence, an anti-VEGF agent (e.g., KH902) or codon-optimized anti-VEGF agent (e.g., KH902) variant-encoding nucleic acid sequence, one or more microRNA binding sites, and a rabbit beta-globin (RBG) poly A sequence. In some embodiments, one or more of the foregoing control sequences is operably linked to a nucleic acid sequence encoding an anti-VEGF agent (e.g., KH902).

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame.

In some embodiments, a transgene comprises a nucleic acid sequence encoding an anti-VEGF agent (e.g., KH902) operably linked to a promoter. A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

Generally, a promoter can be a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the chimeric cytomegalovirus chimeric cytomegalovirus (CMV)/Chicken β-actin (CB) promoter (CBA promotor), the SV40 promoter, the dihydrofolate reductase promoter, the (3-actin promoter, the phosphoglycerol kinase (PGK) promoter, and the EFla promoter [Invitrogen]. In some embodiments, a promoter is an RNA pol II promoter. In some embodiments, a promoter is the chimeric cytomegalovirus chimeric cytomegalovirus (CMV)/Chicken β-actin (CB) promoter (CBA promoter). In some embodiments, a promoter is an RNA pol III promoter, such as U6 or H1.

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the tissue-specific promoter is an eye-specific promoter. Examples of eye-specific promoters include retinoschisin proximal promoter, interphotoreceptor retinoid-binding protein enhancer (RS/IRBPa), rhodopsin kinase (RK), RPE65, and human cone opsin promoter.

In some embodiments, a promoter is a chicken beta-actin (CB) promoter. A chicken beta-actin promoter may be a short chicken beta-actin promoter or a long chicken beta-actin promoter. In some embodiments, a promoter (e.g., a chicken beta-actin promoter) comprises an enhancer sequence, for example a cytomegalovirus (CMV) enhancer sequence. A CMV enhancer sequence may be a short CMV enhancer sequence or a long CMV enhancer sequence. In some embodiments, a promoter comprises a long CMV enhancer sequence and a long chicken beta-actin promoter. In some embodiments, a promoter comprises a short CMV enhancer sequence and a short chicken beta-actin promoter. However, the skilled artisan recognizes that a short CMV enhancer may be used with a long CB promoter, and a long CMV enhancer may be used with a short CB promoter (and vice versa).

An isolated nucleic acid described herein may also contain one or more introns. In some embodiments, at least one intron is located between the promoter/enhancer sequence and the transgene. In some embodiments, an intron is a synthetic or artificial (e.g., heterologous) intron. Examples of synthetic introns include an intron sequence derived from SV-40 (referred to as the SV-40 T intron sequence) and intron sequences derived from chicken beta-actin gene. In some embodiments, a transgene described by the disclosure comprises one or more (1, 2, 3, 4, 5, or more) artificial introns. In some embodiments, the one or more artificial introns are positioned between a promoter and a nucleic acid sequence encoding an anti-VEGF agent (e.g., KH902).

In some embodiments, the transgene described herein comprises a Kozak sequence. A Kozak sequence is a nucleic acid motif comprising a consensus sequence GCC(A/G)CC (SEQ ID NO: 4) that is found in eukaryotic mRNA and plays a role in initiation of protein translation. In some embodiments, the Kozak sequence is positioned between the intron and the transgene encoding the anti-VEGF agent (e.g., KH902).

An isolated nucleic acid described by the disclosure may encode a transgene that further comprises a polyadenylation (poly A) sequence. In some embodiments, a transgene comprises a poly A sequence is a rabbit beta-globin (RBG) poly A sequence,

In some embodiments, the transgene comprises a 3′-untranslated region (3′-UTR). In some embodiments, the disclosure relates to isolated nucleic acids comprising a transgene encoding an anti-VEGF agent (e.g., KH902), and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′-UTR) of a transgene, for example between the last codon of a nucleic acid sequence encoding an anti-VEGF agent (e.g., KH902), and a poly A sequence.

In some embodiments, a transgene comprises one or more (e.g., 1, 2, 3, 4, 5, or more) miRNA binding sites that de-target expression of anti-VEGF agent (e.g., KH902) from immune cells (e.g., antigen presenting cells (APCs), such as macrophages, dendrites, etc.). Incorporation of miRNA binding sites for immune-associated miRNAs may de-target transgene (e.g., KH902) expression from antigen presenting cells and thus reduce or eliminate immune responses (cellular and/or humoral) produced in the subject against products of the transgene, for example as described in US 2018/0066279, the entire contents of which are incorporated herein by reference.

In some aspects, the disclosure relates to isolated nucleic acids comprising a transgene encoding an anti-VEGF agent (e.g., KH902), and one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more GM3S proteins, and a poly A sequence.

In some embodiments, a transgene comprises one or more (e.g., 1, 2, 3, 4, 5, or more) miRNA binding sites that de-target expression of the anti-VEGF agent (e.g., KH902) from liver cells. For example, in some embodiments, a transgene comprises one or more miR-122 binding sites.

In some embodiments, a transgene comprises one or more (e.g., 1, 2, 3, 4, 5, or more) miRNA binding sites that de-target expression of the one or more GM3S proteins from immune cells (e.g., antigen presenting cells (APCs), such as macrophages, dendrites, etc.). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune cell-associated miRNA” is a miRNA preferentially expressed in cells of the immune system, such as an antigen presenting cell (APC). In some embodiments, an immune cell-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune cell-associated miRNA is expressed is a B cell, T cell, Killer T cell, Helper T cell, y6 T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune cell-associated miRNA is selected from: miR-31, miR-106a, miR-125a/b, miR-146a, miR-150, miR-155, miR-181a, miR-223, miR-221, miR-222, let-7i, miR-148, miR-152, miR-126a, miR-142, miR-15, miR-150, miR-155, miR-16, miR-17, miR-18, miR-181 a, miR-19a, miR-19b, miR-20, miR-21a, miR-223, miR-24-3p, miR-29a, miR-29b, miR-29c, miR-302a-3p, miR-30b, miR-33-5p, miR-34a, miR-424, miR-652-3p, miR-652-5p, miR-9-3p, miR-9-5p, miR-92a, and miR-99b-5. In some embodiments, a transgene described herein comprises one or more binding sites for miR-142.

In some embodiments, the isolated nucleic acid comprises inverted terminal repeats. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein (e.g., anti-VEGF agent such as KH902) and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAV5 ITR, and AAV6 ITR. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR, or ΔITR). Without wishing to be bound by any particular theory, a rAAV vector comprising an ITR lacking a functional TRS produces a self-complementary rAAV vector, for example as described by McCarthy (2008) Molecular Therapy 16(10):1648-1656. In some embodiments, vectors described herein comprise one or more AAV ITRs, and at least one ITR is an ITR variant of a known AAV serotype ITR. In some embodiments, the AAV ITR variant is a synthetic AAV ITR (e.g., AAV ITRs that do not occur naturally). In some embodiments, the AAV ITR variant is a hybrid ITR (e.g., a hybrid ITR comprises sequences derived from ITRs of two or more different AAV serotypes).

In some embodiments, an isolated nucleic acid (e.g., a rAAV vector) as described herein comprises, from 5′ to 3′ order: a 5′ AAV ITR, a CMV enhancer, a CBA promoter, an intron (e.g., chicken beta actin intron), a Kozak sequence, a transgene encoding an anti-VEGF agent (e.g., KH902), a rabbit beta-globin poly A, and a 3′ AAV ITR. An exemplary sequence of the isolated nucleic acid sequence is set forth in SEQ ID NO: 2. In some embodiments, the rAAV comprises an isolated nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 2 (Kozak sequence underlined; KH902 coding sequence in bold):

(SEQ ID NO: 2)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGAACTATAGCTA
GTCGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGC
GCGCGGCGGGCGGGGAGTCGCTGCGACGCTGCCTTCGCCCCGTGCCCCGC
TCCGCCGCCGCCTCGCGCCGCCCGCCCCGGCTCTGACTGACCGCGTTACT
CCCACAGGTGAGCGGGCGGGACGGCCCTTCTCCTCCGGGCTGTAATTAGC
GCTTGGTTTAATGACGGCTTGTTTCTTTTCTGTGGCTGCGTGAAAGCCTT
GAGGGGCTCCGGGAGGGCCCTTTGTGCGGGGGGAGCGGCTCGGGGGGTGC
GTGCGTGTGTGTGTGCGTGGGGAGCGCCGCGTGCGGCTCCGCGCTGCCCG
GCGGCTGTGAGCGCTGCGGGCGCGGCGCGGGGCTTTGTGCGCTCCGCAGT
GTGCGCGAGGGGAGCGCGGCCGGGGGCGGTGCCCCGCGGTGCGGGGGGGG
CTGCGAGGGGAACAAAGGCTGCGTGCGGGGTGTGTGCGTGGGGGGGTGAG
CAGGGGGTGTGGGCGCGTCGGTCGGGCTGCAACCCCCCCTGCACCCCCCT
CCCCGAGTTGCTGAGCACGGCCCGGCTTCGGGTGCGGGGCTCCGTACGGG
GCGTGGCGCGGGGCTCGCCGTGCCGGGCGGGGGGTGGCGGCAGGTGGGGG
TGCCGGGCGGGGCGGGGCCGCCTCGGGCCGGGGAGGGCTCGGGGGAGGGG
CGCGGCGGCCCCCGGAGCGCCGGCGGCTGTCGAGGCGCGGCGAGCCGCAG
CCATTGCCTTTTATGGTAATCGTGCGAGAGGGCGCAGGGACTTCCTTTGT
CCCAAATCTGTGCGGAGCCGAAATCTGGGAGGCGCCGCCGCACCCCCTCT
AGCGGGCGCGGGGCGAAGCGGTGCGGCGCCGGCAGGAAGGAAATGGGCGG
GGAGGGCCTTCGTGCGTCGCCGCGCCGCCGTCCCCTTCTCCCTCTCCAGC
CTCGGGGCTGTCCGCGGGGGGACGGCTGCCTTCGGGGGGGACGGGGCAGG
GCGGGGTTCGGCTTCTGGCGTGTGACCGGCGGCTCTAGAGCCTCTGCTAA
CCATGTTCATGCCTTCTTCTTTTTCCTACAGCTCCTGGGCAACGTGCTGG
TTATTGTGCTGTCTCATCATTTTGGCAAAGAATTCGCCACCATGGTCAGC
TACTGGGACACCGGGGTCCTGCTGTGCGCGCTGCTCAGCTGTCTGCTTCT
CACAGGATCTAGTTCCGGAGGTAGACCTTTCGTAGAGATGTACAGTGAAA
TCCCCGAAATTATACACATGACTGAAGGAAGGGAGCTCGTCATTCCCTGC
CGGGTTACGTCACCTAACATCACTGTTACTTTAAAAAAGTTTCCACTTGA
CACTTTGATCCCTGATGGAAAACGCATAATCTGGGACAGTAGAAAGGGCT
TCATCATATCAAATGCAACGTACAAAGAAATAGGGCTTCTGACCTGTGAA
GCAACAGTCAATGGGCATTTGTATAAGACAAACTATCTCACACATCGACA
AACCAATACAATCATAGATGTGGTTCTGAGTCCGTCTCATGGAATTGAAC
TATCTGTTGGAGAAAAGCTTGTCTTAAATTGTACAGCAAGAACTGAACTA
AATGTGGGGATTGACTTCAACTGGGAATACCCTTCTTCGAAGCATCAGCA
TAAGAAACTTGTAAACCGAGACCTAAAAACCCAGTCTGGGAGTGAGATGA
AGAAATTTTTGAGCACCTTAACTATAGATGGTGTAACCCGGAGTGACCAA
GGATTGTACACCTGTGCAGCATCCAGTGGGCTGATGACCAAGAAGAACAG
CACATTTGTCAGGGTCCATGAAAAACCTTTTGTTGCTTTTGGAAGTGGCA
TGGAATCTCTGGTGGAAGCCACGGTGGGGGAGCGTGTCAGAATCCCTGCG
AAGTACCTTGGTTACCCACCCCCAGAAATAAAATGGTATAAAAATGGAAT
ACCCCTTGAGTCCAATCACACAATTAAAGCGGGGCATGTACTGACGATTA
TGGAAGTGAGTGAAAGAGACACAGGAAATTACACTGTCATCCTTACCAAT
CCCATTTCAAAGGAGAAGCAGAGCCATGTGGTCTCTCTGGTTGTGTATGT
CCCACCGGGCCCGGGCGACAAAACTCACACATGCCCACTGTGCCCAGCAC
CTGAACTCCTGGGGGGACCGTCAGTCTTCCTCTTCCCCCCAAAACCCAAG
GACACCCTCATGATCTCCCGGACCCCTGAGGTCACATGCGTGGTGGTGGA
CGTGAGCCACGAAGACCCTGAGGTCAAGTTCAACTGGTACGTGGACGGCG
TGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTG
TACACCCTGCCCCCATCCCGGGATGAGCTGACCAAGAACCAGGTCAGCCT
GACCTGCCTAGTCAAAGGCTTCTATCCCAGCGACATCGCCGTGGAGTGGG
AGAGCAATGGGCAGCCGGAGAACAACTACAAGGCCACGCCTCCCGTGCTG
GACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTC
TGCACAACCACTACACGCAGAAGAGCCTCTCCCTGTCTCCGGGTAAATGA
ACGCGTGGTACCTCTAGAGTCGACCCGGGCGGCCTCGAGGACGGGGTGAA
CTACGCCTGAGGATCCGATCTTTTTCCCTCTGCCAAAAATTATGGGGACA
TCATGAAGCCCCTTGAGCATCTGACTTCTGGCTAATAAAGGAAATTTATT
TTCATTGCAATAGTGTGTTGGAATTTTTTGTGTCTCTCACTCGGAAGCAA
TTCGTTGATCTGAATTTCGACCACCCATAATACCCATTACCCTGGTAGAT
AAGTAGCATGGCGGGTTAATCATTAACTACAAGGAACCCCTAGTGATGGA
GTTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGAG
CGAGCGCGCAG

Also, within the scope of the present disclosure are vectors comprising the isolated nucleic acid described herein. In some embodiments, a vector is a plasmid. In some embodiments, a plasmid comprising an rAAV vector further comprises one or more selection markers. Selection markers are known in the art and include antibiotic resistance markers. In some embodiments, a selection marker comprises a kanamycin resistance marker (e.g., a Neomycin phosphotransferase II (nptII) gene). In some embodiments, a selection marker comprises an ampicillin resistance marker (e.g., a beta-lactamase gene).

An exemplary full vector sequence of pAAV-CBAOKH902 is set forth in SEQ ID NO: 3. In some embodiments, the rAAV vector comprises a nucleic acid sequence at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 3:

(SEQ ID NO: 3)
CTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACCAGGGTAATGGGGATCCTCTAGAAC
TATAGCTAGTCGACATTGATTATTGACTAGTTATTAATAGTAATCAATTACGGGGTCATTAGTT
CATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCATTATG
CCCAGTACATGACCTTATGGGACTTTCCTACTTGGCAGTACATCTACGTATTAGTCATCGCTAT
TACCATGGTCGAGGTGAGCCCCACGTTCTGCTTCACTCTCCCCATCTCCCCCCCCTCCCCACCC
CCAATTTTGTATTTATTTATTTTTTAATTATTTTGTGCAGCGATGGGGGCGGGGGGGGGGGGGG
GGCGCGCGCCAGGCGGGGCGGGGCGGGGCGAGGGGCGGGGCGGGGCGAGGCGGAGAGGTGCGGC
GGCAGCCAATCAGAGCGGCGCGCTCCGAAAGTTTCCTTTTATGGCGAGGCGGCGGCGGCGGCGG
CCCTATAAAAAGCGAAGCGCGCGGCGGGCGGGGAGTCGCTGCGACGCTGCCTTCGCCCCGTGCC
CCGCTCCGCCGCCGCCTCGCGCCGCCCGCCCCGGCTCTGACTGACCGCGTTACTCCCACAGGTG
AGCGGGCGGGACGGCCCTTCTCCTCCGGGCTGTAATTAGCGCTTGGTTTAATGACGGCTTGTTT
CTTTTCTGTGGCTGCGTGAAAGCCTTGAGGGGCTCCGGGAGGGCCCTTTGTGCGGGGGGAGCGG
CTCGGGGGGTGCGTGCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAAGGCTGCGTGCGGGG
TGTGTGCGTGGGGGGGTGAGCAGGGGGTGTGGGCGCGTCGGTCGGGCTGCAACCCCCCCTGCAC
CCCCCTCCCCGAGTTGCTGAGCACGGCCCGGCTTCGGGTGCGGGGCTCCGTACGGGGCGTGGCG
CGGGGCTCGCCGTGCCGGGCGGGGGGTGGCGGCAGGTGGGGGTGCCGGGCGGGGCGGGGCCGCC
TCGGGCCGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTTTGT
CCCAAATCTGTGCGGAGCCGAAATCTGGGAGGCGCCGCCGCACCCCCTCTAGCGGGCGCGGGGC
GAAGCGGTGCGGCGCCGGCAGGAAGGAAATGGGCGGGGAGGGCCTTCGTGCGTCGCCGCGCCGC
CGTCCCCTTCTCCCTCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCCTCTGCTAACCATGT
TCATGCCTTCTTCTTTTTCCTACAGCTCCTGGGCAACGTGCTGGTTATTGTGCTGTCTCATCAT
TTTGGCAAAGAATTCGCCACCATGGTCAGCTACTGGGACACCGGGGTCCTGCTGTGCGCGCTGC
TCAGCTGTCTGCTTCTCACAGGATCTAGTTCCGGAGGTAGACCTTTCGTAGAGATGTACAGTGA
AATCCCCGAAATTATACACATGACTGAAGGAAGGGAGCTCGTCATTCCCTGCCGGGTTACGTCA
CCTAACATCACTGTTACTTTAAAAAAGTTTCCACTTGACACTTTGATCCCTGATGGAAAACGCA
TAATCTGGGACAGTAGAAAGGGCTTCATCATATCAAATGCAACGTACAAAGAAATAGGGCTTCT
GACCTGTGAAGCAACAGTCAATGGGCATTTGTATAAGACAAACTATCTCACACATCGACAAACC
AATACAATCATAGATGTGGTTCTGAGTCCGTCTCATGGAATTGAACTATCTGTTGGAGAAAAGC
TTGTCTTAAATTGTACAGCAAGAACTGAACTAAATGTGGGGATTGACTTCAACTGGGAATACCC
TTCTTCGAAGCATCAGCATAAGAAACTTGTAAACCGAGACCTAAAAACCCAGTCTGGGAGTGAG
ATGAAGAAATTTTTGAGCACCTTAACTATAGATGGTGTAACCCGGAGTGACCAAGGATTGTACA
CCTGTGCAGCATCCAGTGGGCTGATGACCAAGAAGAACAGCACATTTGTCAGGGTCCATGAAAA
ACCTTTTGTTGCTTTTGGAAGTGGCATGGAATCTCTGGTGGAAGCCACGGTGGGGGAGCGTGTC
AGAATCCCTGCGAAGTACCTTGGTTACCCACCCCCAGAAATAAAATGGTATAAAAATGGAATAC
CCCTTGAGTCCAATCACACAATTAAAGCGGGGCATGTACTGACGATTATGGAAGTGAGTGAAAG
AGACACAGGAAATTACACTGTCATCCTTACCAATCCCATTTCAAAGGAGAAGCAGAGCCATGTG
GTCTCTCTGGTTGTGTATGTCCCACCGGGCCCGGGCGACAAAACTCACACATGCCCACTGTGCC
CAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGATGAGC
TGACCAAGAACCAGGTCAGCCTGACCTGCCTAGTCAAAGGCTTCTATCCCAGCGACATCGCCGT
GGAGTGGGAGAGCAATGGGCAGCCGGAGAACAACTACAAGGCCACGCCTCCCGTGCTGGACTCC
GACGGCTCCTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCT
GTCTCCGGGTAAATGAACGCGTGGTACCTCTAGAGTCGACCCGGGCGGCCTCGAGGACGGGGTG
AACTACGCCTGAGGATCCGATCTTTTTCCCTCTGCCAAAAATTATGGGGACATCATGAAGCCCC
TTGAGCATCTGACTTCTGGCTAATAAAGGAAATTTATTTTCATTGCAATAGTGTGTTGGAATTT
TTTGTGTCTCTCACTCGGAAGCAATTCGTTGATCTGAATTTCGACCACCCATAATACCCATTAC
CCTGGTAGATAAGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCCTTAATTAACCTAATTCACT
GGCCGTCGTTTTACAACGTCGTGACTGGGAAAACCCTGGCGTTACCCAACTTAATCGCCTTGCA
GCACATCCCCCTTTCGCCAGCTGGCGTAATAGCGAAGAGGCCCGCACCGATCGCCCTTCCCAAC
AGTTGCGCAGCCTGAATGGCGAATGGGACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTTTC
TTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGTCTATTCTTTTGATT
TATAAGGGATTTTGCCGATTTCGGCCTATTGGTTAAAAAATGAGCTGATTTAACAAAAATTTAA
CGCGAATTTTAACAAAATATTAACGCTTACAATTTAGGTGGCACTTTTCGGGGAAATGTGCGCG
GAACCCCTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGG
TAAGATCCTTGAGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGAC
AGTAAGAGAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTCTAGCTTCC
CGGCAACAATTAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTTTAATTTAA
AAGGATCTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTGTCGGGTTTCGCCAC
CTCTGACTTGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATACCGCTCGCCGC
AGCCGAACGACCGAGCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGGTTTCCCGACTGGAA
AGCGGGCAGTGAGCGCAACGCAATTAATGTGAGTTAGCTCACTCATTAGGCACCCCAGGCTTTA
CACTTTATGCTTCCGGCTCGTATGTTGTGTGGAATTGTGAGCGGATAACAATTTCACACAGGAA
ACAGCTATGACCATGATTACGCCAGATTTAATTAAGGCCTTAATTAGG

In some embodiments, the rAAV comprises an isolated nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 30:

GGTACCTCTAGAGTCGACCCGGGCGGCCTCGAGGACGGGGTGAACTACGCCTGAGGATCCGATC
TTTTTCCCTCTGCCAAAAATTATGGGGACATCATGAAGCCCCTTGAGCATCTGACTTCTGGCTA
ATAAAGGAAATTTATTTTCATTGCAATAGTGTGTTGGAATTTTTTGTGTCTCTCACTCGGAAGC
AATTCGTTGATCTGAATTTCGACCACCCATAATACCCATTACCCTGGTAGATAAGTAGCATGGC
GGGTTAATCATTAACTACAAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCCTC
AGTGAGCGAGCGAGCGCGCAGCCTTAATTAACCTAATTCACTGGCCGTCGTTTTACAACGTCGT
GACTGGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAATGGCGA
ATGGGACGCGCCCTGTAGCGGCGCATTAAGCGCGGCGGGTGTGGTGGTTACGCGCAGCGTGACC
GCTACACTTGCCAGCGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCCCTGA
TAGACGGTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCCGATTTC
GGCCTATTGGTTAAAAAATGAGCTGATTTAACAAAAATTTAACGCGAATTTTAACAAAATATTA
ACGCTTACAATTTAGGTGGCACTTTTCGGGGAAATGTGCGCGGAACCCCTATTTGTTTATTTTT
CTAAATACATTCAAATATGTATCCGCTCATGAGACAATAACCCTGATAAATGCTTCAATAATAT
TGAAAAAGGAAGAGTATGATTGAACAAGATGGATTGCACGCAGGTTCTCCGGCCGCTTGGGTGG
AGAGGCTATTCGGCTATGACTGGGCACAACAGACAATCGGCTGCTCTGATGCCGCCGTGTTCCG
GCTGTCAGCGCAGGGGCGCCCGGTTCTTTTTGTCAAGACCGACCTGTCCGGTGCCCTGAATGAA
CTGCAAGACGAGGCAGCGCGGCTATCGTGGCTGGCCACGACGGGCGTTCCTTGCGCAGCTGTGC
TCGACGTTGTCACTGAAGCGGGAAGGGACTGGCTGCTATTGGGCGAAGTGCCGGGGCAGGATCT
CCTGTCATCTCACCTTGCTCCTGCCGAGAAAGTATCCATCATGGCTGATGCAATGCGGCGGCTG
CATACGCTTGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAGGGGCTCGC
GCCAGCCGAACTGTTCGCCAGGCTCAAGGCGAGCATGCCCGACGGCGAGGATCTCGTCGTGACC
CATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGACTGTCAGACCAAGTTTACTCAT
ATATACTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGACCCCGTA
GAAAAGATCAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCTTTTTCCGAA
GGTAACTGGCTTCAGCAGAGCGCAGATACCAAATACTGTTCTTCTAGTGTAGCCGTAGTTAGGC
CACCACTTCAAGAACTCTGTAGCACCGCCTACATACCTCGCTCTGCTAATCCTGTTACCAGTGG
CTGCTGCCAGTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAGCTTCCAGGGGG
AAACGCCTGGTATCTTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGCCTTTTTACGGTTCC
TGGCCTTTTGCTGGCCTTTTGCTCACATGTTCTTTCCTGCGTTATCCCCTGATTCTGTGGATAA
CCGTATTACCGCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCGCGTTGGCCGA
TTCATTAATGCAGCTGGCACGACAGGTTTCCCGACTGGAAAGCGGGCAGTGAGCGCAACGCAAT
TAATGTGAGTTAGCTCACTCATTAGGCACCCCAGGCTTTACACTTTATGCTTCCGGCTCGTATG
TTGTGTGGAATTGTGAGCGGATAACAATTTCACACAGGAAACAGCTATGACCATGATTACGCCA
GATTTAATTAAGGCCTTAATTAGGCTGCGCGCTCGCTCGCTCACTGAGGCCGCCCGGGCAAAGC
CCGGGCGTCGGGCGACCTTTGGTCGCCCGGCCTCAGTGAGCGAGCGAGCGCGCAGAGAGGGAGT
GGCCAACTCCATCACTAGGGGTTCCTTGTAGTTAATGATTAACCCGCCATGCTACTTATCTACC
AGGGTAATGGGGATCCTCTAGAACTATAGCTAGTCGACATTGATTATTGACTAGTTATTAATAG
TAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACG
GTAAATGGCCCGCCTGGCATTATGCCCAGTACATGACCTTATGGGACTTTCCTACTTGGCAGTA
CATCTACGTATTAGTCATCGCTATTACCATGGTCGAGGTGAGCCCCACGTTCTGCTTCACTCTC
CCCATCTCCCCCCCCTCCCCACCCCCAATTTTGTATTTATTTATTTTTTAATTATTTTGTGCAG
CGATGGGGGCGGGGGGGGGGGGGGGGCGCGCGCCAGGCGGGGCGGGGCGGGGCGAGGGGCGGGG
CGGGGCGAGGCGGAGAGGTGCGGCGGCAGCCAATCAGAGCGGCGCGCTCCGAAAGTTTCCTTTT
ATGGCGAGGCGGCGGCGGCGGCGGCCCTATAAAAAGCGAAGCGCGCGGCGGGCGGGAGTCGCTG
CGCGCTGCCTTCGCCCCGTGCCCCGCTCCGCCGCCGCCTCGCGCCGCCCGCCCCGGCTCTGACT
GACCGCGTTACTCCCACAGGTGAGCGGGCGGGACGGCCCTTCTCCTCCGGGCTGTAATTAGCGC
TTGGTTTAATGACGGCTTGTTTCTTTTCTGTGGCTGCGTGAAAGCCTTGAGGGGCTCCGGGAGG
GCCCTTTGTGCGGGGGGAGCGGCTCGGGGGGTGCGTGCGTGTGTGTGTGCGTGGGGAGCGCCGC
GTGCGGCTCCGCGCTGCCCGGCGGCTGTGAGCGCTGCGGGCGCGGCGCGGGGCTTTGTGCGCTC
CGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGT
CGGGCTGCAACCCCCCCTGCACCCCCCTCCCCGAGTTGCTGAGCACGGCCCGGCTTCGGGTGCG
GGGCTCCGTACGGGGCGTGGCGCGGGGCTCGCCGTGCCGGGCGGGGGGTGGCGGCAGGTGGGGG
TGCCGGGCGGGGCGGGGCCGCCTCGGGCCGGGGAGGGCTCGGGGGAGGGGCGCGGCGGCCCCCG
GAGCGCCGGCGGCTGTCGAGGCGCGGCGAGCCGCAGCCATTGCCTTTTATGGTAATCGTGCGAG
AGGGCGCAGGGACTTCCTTTGTCCCAAATCTGTGCGGAGCCGAAATCTGGGAGGCGCCGCCGCA
CCCCCTCTAGCGGGCGCGGGGCGAAGCGGTGCGGCGCCGGCAGGAAGGAAATGGGCGGGGAGGG
CCTTCGTGCGTCGCCGCGCCGCCGTCCCCTTCTCCCTCTCCAGCCTCGGGGCTGTCCGCGGGGG
GACGGCTGCCTTCGGGGGGGACGGGGCAGGGCGGGGTTCGGCTTCTGGCGTGTGACCGGCGGCT
CTAGAGCCTCTGCTAACCATGTTCATGCCTTCTTCTTTTTCCTACAGCTCCTGGGCAACGTGCT
GGTTATTGTGCTGTCTCATCATTTTGGCAAAGAATTCGCCACCATGGTCAGCTACTGGGACACC
GGGGTCCTGCTGTGCGCGCTGCTCAGCTGTCTGCTTCTCACAGGATCTAGTTCCGGAGGTAGAC
CTTTCGTAGAGATGTACAGTGAAATCCCCGAAATTATACACATGACTGAAGGAAGGGAGCTCGT
CATTCCCTGCCGGGTTACGTCACCTAACATCACTGTTACTTTAAAAAAGTTTCCACTTGACACT
TTGATCCCTGATGGAAAACGCATAATCTGGGACAGTAGAAAGGGCTTCATCATATCAAATGCAA
CGTACAAAGAAATAGGGCTTCTGACCTGTGAAGCAACAGTCAATGGGCATTTGTATAAGACAAA
CTATCTCACACATCGACAAACCAATACAATCATAGATGTGGTTCTGAGTCCGTCTCATGGAATT
GAACTATCTGTTGGAGAAAAGCTTGTCTTAAATTGTACAGCAAGAACTGAACTAAATGTGGGGA
TTGACTTCAACTGGGAATACCCTTCTTCGAAGCATCAGCATAAGAAACTTGTAAACCGAGACCT
AAAAACCCAGTCTGGGAGTGAGATGAAGAAATTTTTGAGCACCTTAACTATAGATGGTGTAACC
CGGAGTGACCAAGGATTGTACACCTGTGCAGCATCCAGTGGGCTGATGACCAAGAAGAACAGCA
CATTTGTCAGGGTCCATGAAAAACCTTTTGTTGCTTTTGGAAGTGGCATGGAATCTCTGGTGGA
AGCCACGGTGGGGGAGCGTGTCAGAATCCCTGCGAAGTACCTTGGTTACCCACCCCCAGAAATA
AAATGGTATAAAAATGGAATACCCCTTGAGTCCAATCACACAATTAAAGCGGGGCATGTACTGA
CGATTATGGAAGTGAGTGAAAGAGACACAGGAAATTACACTGTCATCCTTACCAATCCCATTTC
AAAGGAGAAGCAGAGCCATGTGGTCTCTCTGGTTGTGTATGTCCCACCGGGCCCGGGCGACAAA
ACTCACACATGCCCACTGTGCCCAGCACCTGAACTCCTGGGGGGACCGTCAGTCTTCCTCTTCC
CCCCAAAACCCAAGGACACCCTCATGATCTCCCGGACCCCTGAGGTCACATGCGTGGTGGTGGA
CGTGAGCCACGAAGACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCAAGGTCTCCAACAAAGCCCTCCC
AGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTAGTCAAAGGCT
TCTATCCCAGCGACATCGCCGTGGAGTGGGAGAGCAATGGGCAGCCGGAGAACAACTACAAGGC
CACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCTCTGCACAACCACT
ACACGCAGAAGAGCCTCTCCCTGTCTCCGGGTAAATGAACGCGT

In some embodiments, the rAAV comprises an isolated nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 31:

CAGACATGATAAGATACATTGATGAGTTTGGACAAACCACAACTAGAATGCAGTGAAAAAAATG
CTTTATTTGTGAAATTTGTGATGCTATTGCTTTATTTGTAACCATTATAAGCTGCAATAAACAA
GTTAACAACAACAATTGCATTCATTTTATGTTTCAGGTTCAGGGGGAGATGTGGGAGGTTTTTT
AAAGCAAGTAAAACCTCTACAAATGTGGTACTCGAGGAAGCAATTCGTTGATCTGAATTTCGAC
CACCCATAATACCCATTACCCTGGTAGATAAGTAGCATGGCGGGTTAATCATTAACTACAAGGA
ACCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGCAGCC
TTAATTAACCTAATTCACTGGCCGTCGTTTTACAACGTCGTGACTGGGAAAACCCTGGCGTTAC
CCAACTTAATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGGGACGCGCCCTGTAGCGGCG
CATTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAGCT
CTAAATCGGGGGCTCCCTTTAGGGTTCCGATTTAGTGCTTTACGGCACCTCGACCCCAAAAAAC
TTGATTAGGGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCAACCCTATC
TCGGTCTATTCTTTTGATTTATAAGGGATTTTGCCGATTTCGGCCTATTGGTTAAAAAATGAGC
TGATTTAACAAAAATTTAACGCGAATTTTAACAAAATATTAACGCTTACAATTTAGGTGGCACT
TTTCGGGGAAATGTGCGCGGAACCCCTATTTGTTTATTTTTCTAAATACATTCAAATATGTATC
CGCTCATGAGACAATAACCCTGATAAATGCTTCAATAATATTGAAAAAGGAAGAGTATGATTGA
ACAAGATGGATTGCACGCAGGTTCTCCGGCCGCTTGGGTGGAGAGGCTATTCGGCTATGACTGG
GCACAACAGACAATCGGCTGCTCTGATGCCGCCGTGTTCCGGCTGTCAGCGCAGGGGCGCCCGG
TTCTTTTTGTCAAGACCGACCTGTCCGGTGCCCTGAATGAACTGCAAGACGAGGCAGCGCGGCT
ATCGTGGCTGGCCACGACGGGCGTTCCTTGCGCAGCTGTGCTCGACGTTGTCACTGAAGCGGGA
AGGGACTGGCTGCTATTGGGCGAAGTGCCGGGGCAGGATCTCCTGTCATCTCACCTTGCTCCTG
CCGAGAAAGTATCCATCATGGCTGATGCAATGCGGCGGCTGCATACGCTTGATCCGGCTACCTG
CCCATTCGACCACCAAGCGAAACATCGCATCGAGCGAGCACGTACTCGGATGGAAGCCGGTCTT
GTCGATCAGGATGATCTGGACGAAGAGCATCAGGGGCTCGCGCCAGCCGAACTGTTCGCCAGGC
TCAAGGCGAGCATGCCCGACGGCGAGGATCTCGTCGTGACCCATGGCGATGCCTGCTTGCCGAA
TATCATGGTGGAAAATGGCCGCTTTTCTGGATTCATCGACTGTGGCCGGCTGGGTGTGGCGGAC
CGCTATCAGGACATAGCGTTGGCTACCCGTGATATTGCTGAAGAGCTTGGCGGCGAATGGGCTG
ACCGCTTCCTCGTGCTTTACGGTATCGCCGCTCCCGATTCGCAGCGCATCGCCTTCTATCGCCT
TCTTGACGAGTTCTTCTGACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCC
CTTAACGTGAGTTTTCGTTCCACTGAGCGTCAGACCCCGTAGAAAAGATCAAAGGATCTTCTTG
AGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGC
AGATACCAAATACTGTTCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATACCTACAGCG
TGAGCTATGAGAAAGCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGCACGAC
AGGTTTCCCGACTGGAAAGCGGGCAGTGAGCGCAACGCAATTAATGTGAGTTAGCTCACTCATT
AGGCACCCCAGGCTTTACACTTTATGCTTCCGGCTCGTATGTTGTGTGGAATTGTGAGCGGATA
ACAATTTCACACAGGAAACAGCTATGACCATGATTACGCCAGATTTAATTAAGGCCTTAATTAG
GCTGCGCGCTCGCTCGCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAACTCCATCACTAGGGGTT
CCTTGTAGTTAATGATTAACCCGCCATGCTACTTATCTACCAGGGTAATGGGGATCCGGAGTTC
CGCGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTTTTG
GCAGTACACCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAACAACT
CCGCCCCATTGACGCAAATGGGCGGTAGGCGTGTACGGTGGGAGGTCTATATAAGCAGAGCTCG
TTTAGTGAACCGTCAGATCGCCTGGAGACGCCATCCACGCTGTTTTGACCTCCATAGAAGACAC
CGACTCTACTAGAGGATCTATTTCCGGTGAATTCGCCACCATGGTGAGCTACTGGGACACCGGC
GTGCTGCTGTGCGCCCTGCTGAGCTGCCTGCTGCTGACCGGCAGCAGCAGCGGCGGCAGACCTT
TCGTGGAGATGTACTCCGAGATCCCCGAGATCATCCACATGACCGAGGGCAGGGAGCTCGTGAT
CCCCTGCAGAGTGACCAGCCCCAACATCACCGTGACCCTGAAGAAGTTCCCCCTGGACACCCTG
ATCCCCGACGGCAAGAGAATCATCTGGGACAGCAGAAAGGGCTTCATCATCTCCAACGCCACCT
ACAAGGAGATCGGCCTGCTGACCTGCGAGGCCACCGTGAACGGCCACCTGTACAAGACCAATTA
CCTGACCCACAGGCAGACCAATACCATCATCGACGTGGTGCTGTCCCCCAGCCACGGCATCGAG
CTGAGCGTGGGCGAGAAGCTGGTGCTGAACTGCACCGCCAGGACCGAGCTGAACGTGGGGATCG
ATTTTAACTGGGAGTACCCCAGCAGCAAGCACCAGCACAAGAAGCTGGTGAATAGGGACCTGAA
AACCCAGAGCGGAAGCGAGATGAAGAAGTTTCTGAGCACCCTGACCATCGACGGCGTGACCCGG
AGCGACCAGGGCCTGTACACCTGCGCCGCCTCCAGCGGCCTGATGACTAAGAAGAACAGCACCT
TTGTGCGGGTGCACGAGAAGCCCTTCGTGGCCTTCGGCAGCGGGATGGAGTCTCTGGTGGAGGC
TACCGTGGGCGAGAGAGTGAGAATCCCCGCCAAGTACCTGGGCTACCCCCCTCCTGAGATCAAG
TGGTATAAGAACGGCATCCCTCTGGAGTCCAACCACACCATCAAGGCAGGCCACGTGCTGACCA
TCATGGAAGTGAGCGAGAGGGACACCGGCAACTACACCGTGATCCTGACCAACCCCATCTCCAA
GGAGAAGCAGAGCCACGTGGTGAGCCTGGTGGTGTACGTGCCTCCAGGGCCTGGCGATAAGACC
CACACATGCCCCCTGTGCCCCGCCCCCGAGCTGCTGGGCGGACCAAGCGTGTTCCTGTTCCCAC
CCAAGCCTAAGGACACCCTGATGATCAGCCGGACCCCCGAGGTGACCTGCGTGGTGGTGGATGT
GAGCCACGAGGATCCAGAGGTGAAGTTTAACTGGTATGTGGACGGCGTGGAGGTGCACAACGCC
AAGACCAAGCCCAGGGAGGAGCAGTACAACAGCACCTACAGAGTGGTGAGCGTGCTGACCGTGC
TGCACCAGGACTGGCTGAACGGCAAGGAGTACAAGTGCAAGGTGAGCAACAAGGCCCTGCCCGC
CCCTATCGAGAAAACCATCAGCAAGGCCAAGGGCCAGCCCCGCGAGCCCCAGGTGTACACACTG
CCCCCTAGCCGCGACGAGCTGACCAAGAACCAGGTGTCCCTGACCTGCCTGGTGAAGGGCTTCT
ACCCCAGCGACATCGCCGTGGAGTGGGAGAGCAACGGCCAGCCCGAGAACAACTACAAGGCCAC
CCCCCCTGTGCTGGACTCCGACGGCAGCTTCTTCCTGTACAGCAAGCTGACCGTGGACAAGTCC
CGCTGGCAGCAGGGCAACGTGTTCAGCTGTAGCGTGATGCACGAGGCCCTGCACAACCACTACA
CCCAGAAGTCCCTGAGCCTGAGCCCCGGCAAGTGAACGCGT

In some embodiments, the rAAV comprises an isolated nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the nucleic acid sequence as set forth in SEQ ID NO: 32

AATTCGATATCAAGCTTATCGATAATCAACCTCTGGATTACAAAATTTGTGAAAGATTGACTGG
TATTCTTAACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTGACGCAAC
CCCCACTGGTTGGGGCATTGCCACCACCTGTCAGCTCCTTTCCGGGACTTTCGCTTTCCCCCTC
CCTATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCCTG
TGTTGCCACCTGGATTCTGCGCGGGACGTCCTTCTGCTACGTCCCTTCGGCCCTCAATCCAGCG
GACCTTCCTTCCCGCGGCCTGCTGCCGGCTCTGCGGCCTCTTCCGCGTCTTCGCCTTCGCCCTC
AGACGAGTCGGATCTCCCTTTGGGCCGCCTCCCCGCATCGATACCGTCGACCCGGGCGGCCGCT
TCGAGCAGACATGATAAGATACATTGATGAGTTTGGACAAACCACAACTAGAATGCAGTGAAAA
AAATGCTTTATTTGTGAAATTTGTGATGCTATTGCTTTATTTGTAACCATTATAAGCTGCAATA
AACAAGTTAACAACAACAATTGCATTCATTTTATGTTTCAGGTTCAGGGGGAGATGTGGGAGGT
TTTTTAAAGCAAGTAAAACCTCTACAAATGTGGTACTCGAGGAAGCAATTCGTTGATCTGAATT
TCGACCACCCATAATACCCATTACCCTGGTAGATAAGTAGCATGGCGGGTTAATCATTAACTAC
AAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCG
CAGCCTTAATTAACCTAATTCACTGGCCGTCGTTTTACAACGTCGTGACTGGGAAAACCCTGGC
GTTACCCAACTTAATCGCCTTGCAGCACATCCCCCTTTCGCCAGCTGGCGTAATAGCGAAGAGG
CCCGCACCGATCGCCCTTCCCAACAGTTGCGCAGCCTGAATGGCGAATGGGACGCGCCCTGTAG
CGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTC
AAGCTCTAAATCGGGGGCTCCCTTTAGGGTTCCGATTTAGTGCTTTACGGCACCTCGACCCCAA
AAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAAAAAA
TGAGCTGATTTAACAAAAATTTAACGCGAATTTTAACAAAATATTAACGCTTACAATTTAGGTG
GCACTTTTCGGGGAAATGTGCGCGGAACCCCTATTTGTTTATTTTTCTAAATACATTCAAATAT
GTATCCGCTCATGAGACAATAACCCTGATAAATGCTTCAATAATATTGAAAAAGGAAGAGTATG
ATTGAACAAGATGGATTGCACGCAGGTTCTCCGGCCGCTTGGGTGGAGAGGCTATTCGGCTATG
ACTGGGCACAACAGACAATCGGCTGCTCTGATGCCGCCGTGTTCCGGCTGTCAGCGCAGGGGCG
CCCGGTTCTTTTTGTCAAGACCGACCTGTCCGGTGCCCTGAATGAACTGCAAGACGAGGCAGCG
CGGCTATCGTGGCTGGCCACGACGGGCGTTCCTTGCGCAGCTGTGCTCGACGTTGTCACTGAAG
CGGGAAGGGACTGGCTGCTATTGGGCGAAGTGCCGGGGCAGGATCTCCTGTCATCTCACCTTGC
TCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCG
GTCTTGTCGATCAGGATGATCTGGACGAAGAGCATCAGGGGCTCGCGCCAGCCGAACTGTTCGC
CAGGCTCAAGGCGAGCATGCCCGACGGCGAGGATCTCGTCGTGACCCATGGCGATGCCTGCTTG
CCGAATATCATGGTGGAAAATGGCCGCTTTTCTGGATTCATCGACTGTGGCCGGCTGGGTGTGG
CGGACCGCTATCAGGACATAGCGTTGGCTACCCGTGATATTGCTGAAGAGCTTGGCGGCGAATG
GGCTGACCGCTTCCTCGTGCTTTACGGTATCGCCGCTCCCGATTCGCAGCGCATCGCCTTCTAT
CGCCTTCTTGACGAGTTCTTCTGACTGTCAGACCAAGTTTACTCATATATACTTTAGATTGATT
TAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGCAG
AGCGCAGATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCCTGGTATCTTTA
TAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAGTTAGCTCAC
TCATTAGGCACCCCAGGCTTTACACTTTATGCTTCCGGCTCGTATGTTGTGTGGAATTGTGAGC
GGATAACAATTTCACACAGGAAACAGCTATGACCATGATTACGCCAGATTTAATTAAGGCCTTA
ATTAGGCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAACTCCATCACTAG
GGGTTCCTTGTAGTTAATGATTAACCCGCCATGCTACTTATCTACCAGGGTAATGGGGATCCGG
AGTTCCGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAA
TGGGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTGATGCGG
TTTTGGCAGTACACCAATGGGCGTGGATAGCGGTTTGACTCACGGGGATTTCCAAGTCTCCACC
CCATTGACGTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATATAAGCAGA
GCTCGTTTAGTGAACCGTCAGATCGCCTGGAGACGCCATCCACGCTGTTTTGACCTCCATAGAA
GACACCGACTCTACTAGAGGATCTATTTCCGGTGAATTCGCCACCATGGTGAGCTACTGGGACA
CCGGCGTGCTGCTGTGCGCCCTGCTGAGCTGCCTGCTGCTGACCGGCAGCAGCAGCGGCGGCAG
ACCTTTCGTGGAGATGTACTCCGAGATCCCCGAGATCATCCACATGACCGAGGGCAGGGAGCTC
GTGATCCCCTGCAGAGTGACCAGCCCCAACATCACCGTGACCCTGAAGAAGTTCCCCCTGGACA
CCCTGATCCCCGACGGCAAGAGAATCATCTGGGACAGCAGAAAGGGCTTCATCATCTCCAACGC
CACCTACAAGGAGATCGGCCTGCTGACCTGCGAGGCCACCGTGAACGGCCACCTGTACAAGACC
AATTACCTGACCCACAGGCAGACCAATACCATCATCGACGTGGTGCTGTCCCCCAGCCACGGCA
TCGAGCTGAGCGTGGGCGAGAAGCTGGTGCTGAACTGCACCGCCAGGACCGAGCTGAACGTGGG
GATCGATTTTAACTGGGAGTACCCCAGCAGCAAGCACCAGCACAAGAAGCTGGTGAATAGGGAC
CTGAAAACCCAGAGCGGAAGCGAGATGAAGAAGTTTCTGAGCACCCTGACCATCGACGGCGTGA
CCCGGAGCGACCAGGGCCTGTACACCTGCGCCGCCTCCAGCGGCCTGATGACTAAGAAGAACAG
CACCTTTGTGCGGGTGCACGAGAAGCCCTTCGTGGCCTTCGGCAGCGGGATGGAGTCTCTGGTG
GAGGCTACCGTGGGCGAGAGAGTGAGAATCCCCGCCAAGTACCTGGGCTACCCCCCTCCTGAGA
TCAAGTGGTATAAGAACGGCATCCCTCTGGAGTCCAACCACACCATCAAGGCAGGCCACGTGCT
GACCATCATGGAAGTGAGCGAGAGGGACACCGGCAACTACACCGTGATCCTGACCAACCCCATC
TCCAAGGAGAAGCAGAGCCACGTGGTGAGCCTGGTGGTGTACGTGCCTCCAGGGCCTGGCGATA
AGACCCACACATGCCCCCTGTGCCCCGCCCCCGAGCTGCTGGGCGGACCAAGCGTGTTCCTGTT
CCCACCCAAGCCTAAGGACACCCTGATGATCAGCCGGACCCCCGAGGTGACCTGCGTGGTGGTG
GATGTGAGCCACGAGGATCCAGAGGTGAAGTTTAACTGGTATGTGGACGGCGTGGAGGTGCACA
ACGCCAAGACCAAGCCCAGGGAGGAGCAGTACAACAGCACCTACAGAGTGGTGAGCGTGCTGAC
CGTGCTGCACCAGGACTGGCTGAACGGCAAGGAGTACAAGTGCAAGGTGAGCAACAAGGCCCTG
CCCGCCCCTATCGAGAAAACCATCAGCAAGGCCAAGGGCCAGCCCCGCGAGCCCCAGGTGTACA
CACTGCCCCCTAGCCGCGACGAGCTGACCAAGAACCAGGTGTCCCTGACCTGCCTGGTGAAGGG
CTTCTACCCCAGCGACATCGCCGTGGAGTGGGAGAGCAACGGCCAGCCCGAGAACAACTACAAG
GCCACCCCCCCTGTGCTGGACTCCGACGGCAGCTTCTTCCTGTACAGCAAGCTGACCGTGGACA
AGTCCCGCTGGCAGCAGGGCAACGTGTTCAGCTGTAGCGTGATGCACGAGGCCCTGCACAACCA
CTACACCCAGAAGTCCCTGAGCCTGAGCCCCGGCAAGTGAACGCGT

In some embodiments, the anti-VEGF agent (e.g., KH902) described herein can be delivered to a subject via a non-viral platform. In some embodiments, the anti-VEGF agent (e.g., KH902) described herein can be delivered to a subject via closed-ended linear duplex DNA (ceDNA). Delivery of a transgene (e.g., anti-VEGF agent such as KH902) has been described previously, see e.g., WO2017152149, the entire contents of which are incorporated herein by reference. In some embodiments, the nucleic acids having asymmetric terminal sequences (e.g., asymmetric interrupted self-complementary sequences) form closed-ended linear duplex DNA structures (e.g., ceDNA) that, in some embodiments, exhibit reduced immunogenicity compared to currently available gene delivery vectors. In some embodiments, ceDNA behaves as linear duplex DNA under native conditions and transforms into single-stranded circular DNA under denaturing conditions. Without wishing to be bound by any particular theory, ceDNA are useful, in some embodiments, for the delivery of a transgene (e.g., anti-VEGF agent such as KH902) to a subject.

AAV-Mediated Delivery of a Transgene to Ocular Tissue

Aspects of the instant disclosure relate to compositions comprising a recombinant AAV comprising a capsid protein and a nucleic acid encoding a transgene, wherein the transgene comprises a nucleic acid sequence encoding an anti-VEGF agent (e.g., KH902). In some embodiments, the nucleic acid further comprises AAV ITRs.

The isolated nucleic acids, vectors, rAAVs, and compositions comprising the isolated nucleic acid described herein, the vectors described herein, or the rAAV described herein of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, the subject is a human.

In some embodiments, administration of an isolated nucleic and/or an rAAV as described herein result in delivery of the transgene (e.g., KH902) to ocular tissue. Delivery of the rAAVs to a mammalian subject may be by, for example, intraocular injection, subretinal injection, topical administration (e.g., an eye drop), or by injection into the eye of the mammalian subject to ocular tissues (e.g., intravitreal injection, or intrastromal injection). As used herein, “ocular tissues” refers to any tissue derived from or contained in the eye. Non-limiting examples of ocular tissues include neurons, retina (e.g., photoreceptor cells), sclera, choroid, retina, vitreous body, macula, fovea, optic disc, lens, pupil, iris, aqueous fluid, cornea (e.g., keratocytes, corneal endothelial cells, corneal basal cells, corneal wing cells, and corneal squamous cells), conjunctiva ciliary body, and optic nerve. The retina is located in the posterior of the eye and comprises photoreceptor cells. These photoreceptor cells (e.g., rods, cones) confer visual acuity by discerning color, as well as contrast in the visual field. In some embodiments, administration of an isolated nucleic and/or an rAAV as described herein result in delivery of the transgene (e.g., KH902) to the cornea. In some embodiments, administration of an isolated nucleic and/or an rAAV as described herein result in delivery of the transgene (e.g., KH902) to keratocytes of the cornea.

Alternatively, delivery of the rAAVs to a mammalian subject may be by intramuscular injection or by administration into the bloodstream of the mammalian subject. Administration into the bloodstream may be by injection into a vein, an artery, or any other vascular conduit. Non-limiting exemplary methods of intramuscular administration of the rAAV include Intramuscular (IM) Injection and Intravascular Limb Infusion. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, an rAAV or a composition (e.g., composition containing the isolated nucleic acid or the rAAV) as described in the disclosure is administered by intravitreal injection. In some embodiments, an rAAV or a composition (e.g., composition containing the isolated nucleic acid or the rAAV) as described in the disclosure is administered by intraocular injection. In some embodiments, an rAAV or a composition (e.g., composition containing the isolated nucleic acid or the rAAV) as described in the disclosure is administered by subretinal injection. In some embodiments, an rAAV or a composition (e.g., composition containing the isolated nucleic acid or the rAAV) as described in the disclosure is administered by intravenous injection. In some embodiments, an rAAV or a composition (e.g., composition containing the isolated nucleic acid or the rAAV) as described in the disclosure is administered by intramuscular injection. In some embodiments, an rAAV or a composition (e.g., composition containing the isolated nucleic acid or the rAAV) as described in the disclosure is administered by intratumoral injection.

In some embodiments, administration of an isolated nucleic and/or an rAAV as described herein results in inhibition of VEGF (e.g., VEGF activity). In some embodiments, administration of an isolated nucleic acid and/or an rAAV as described herein results in inhibition of VEGF (e.g., VEGF activity) in ocular tissue. The extent of VEGF inhibition can be measured by any suitable known method (e.g., HUVEC angiogenesis assay, retinal vascular development assay, retinal edema assay, laser damage-induced choroidal neovascular (CNVs), alkali-burn injury model, or suture induced CoNV model, etc.). In some embodiments, VEGF (e.g., VEGF activity) activity in subjects received anti-VEGF agent (e.g., injected with an isolated nucleic acid and/or a rAAV described herein) is inhibited by at least 2%, at least 5%, at least 10%, at least 15%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 100% compared to an uninjected subject, or the same subject before receiving the anti-VEGF agent. In some embodiments, the VEGF (e.g., VEGF activity) in an uninjected subject, or a subject prior to receiving an anti-VEGF agent is by at least 2%, at least 5%, at least 10%, at least 15%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 100%, at least 1-fold, at least 2-fold at least 3-fold, at least 4-fold, at least 5-fold, at least 6-fold, at least 7-fold, at least 8-fold, at least 9-fold, at least 10-fold, at least 10 to 50-fold (e.g., 10-fold, 20-fold, 30-fold, 40-fold, or 50-fold), at least 50 to 100-fold (e.g., 50-fold, 60-fold, 70-fold, 80-fold, 90-fold or 100-fold) or more higher compared to a subject received anti-VEGF agent administration (e.g., injected with an isolated nucleic acid and/or a rAAV described herein). In some embodiments, administration of an anti-VEGF agent (e.g., an isolated nucleic acid and/or a rAAV described herein) result in inhibition of VEGF (e.g., VEGF activity) for longer than 1 day, longer than 2 days, longer than 3 days, longer than 4 days, longer than 5 days, longer than 6 days, longer than 7 days, longer than 1 week (e.g., 8 days, 9 days, 10 days, 11 days, 12 days, 13 days, or 14 days), longer than 2 weeks (e.g., 15 days, 16 days, 17 days, 18 days, 19 days, 20 days, or 21 days), longer than 3 weeks week (e.g., 22 days, 23 days, 24 days, 25 days, 25 days, 27 days, or 28 days), longer than 4 weeks (e.g., 29 days, 30 days, 40 days, 50 days, 60 days, 100 days or more), longer than 1 month (e.g., 5 weeks, 6 weeks, 7 weeks, 8 weeks, 9 weeks, 10 weeks, or more), longer than 2 months (e.g., between 2 months to 2.5 months, between 2 months to 3 months, between 2 months to 4 months, between 2 months to 5 months, between 2 months to 6 months, between 2 months to 7 months, between 2 months to 8 months, between 2 months to 9 months, between 2 months to 10 months, between 2 months to 11 months, between 2 months to 12 months), longer than 3 months (e.g., between 3 months to 4 months, between 3 months to 5 months, between 3 months to 6 months, between 3 months to 7 months, between 3 months to 8 months, between 3 months to 9 months, between 3 months to 10 months, between 3 months to 11 months, between 3 months to 12 months), longer than 4 months (e.g., between 4 months to 5 months, between 4 months to 6 months, between 4 months to 7 months, between 4 months to 8 months, between 4 months to 9 months, between 4 months to 10 months, between 4 months to 11 months, between 4 months to 12 months), longer than 5 months (e.g., between 5 months to 6 months, between 5 months to 7 months, between 5 months to 8 months, between 5 months to 9 months, between 5 months to 10 months, between 5 months to 11 months, between 5 months to 12 months), longer than 6 months (e.g., between 6 months to 7 months, between 6 months to 8 months, between 6 months to 9 months, between 6 months to 10 months, between 6 months to 11 months, between 6 months to 12 months), longer than 7 months (e.g., between 7 months to 8 months, between 7 months to 9 months, between 7 months to 10 months, between 7 months to 11 months, between 7 months to 12 months), longer than 8 months (e.g., between 8 months to 9 months, between 8 months to 10 months, between 8 months to 11 months, between 8 months to 12 months), longer than 9 months (e.g., between 9 months to 10 months, between 9 months to 11 months, between 9 months to 12 months), longer than 10 months (e.g., between 10 months to 11 months, between 11 months to 12 months), longer than 11 months (e.g., between 11 months to 12 months), longer than 12 months (e.g., 12 to 15 months, 12-18 months, 12-21 months, 12-2 months), longer than 1 year (e.g., 1 to 1.5 years), longer than 2 year, longer than 3 year, longer than 4 year, longer than 5 year, longer than 10 years, longer than 15 years, longer than 20 years or more.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs or the compositions (e.g., composition containing the isolated nucleic acid or the rAAV described herein) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intravitreal delivery to the eye), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

An effective amount of rAAVs or composition (e.g., composition containing the isolated nucleic acid or the rAAV described herein) is an amount sufficient to target infect an animal, target a desired tissue (e.g., muscle tissue, ocular tissue, etc.). In some embodiments, an effective amount of an rAAV is administered to the subject during a pre-symptomatic stage of degenerative disease. In some embodiments, a subject is administered an rAAV or composition after exhibiting one or more signs or symptoms of degenerative disease. In some embodiments, the effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some embodiments, an effective amount of an rAAV ranges between 1×10⁹ and 1×10¹⁴ genome copies of the rAAV. In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of about 10¹² to 10¹⁴ rAAV genome copies is appropriate. In some embodiments, a dosage of about 10¹³ to 10¹⁴ rAAV genome copies is appropriate. In some embodiments, a dosage of about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 1×10¹³, about 1.1×10¹³, about 1.2×10¹³, about 1.3×10¹³, about 1.4×10¹³, about 1.5×10¹³, about 1.6×10¹³, about 1.7×10¹³, about 1.8×10¹³, about 1.9×10¹³, or about 2.0×10¹⁴ vector genome (vg) copies per kilogram (kg) of body weight is appropriate. In some embodiments, a dosage of between about 4×10¹² to 2×10¹³ rAAV genome copies is appropriate. In some embodiments a dosage of about 1.5×10¹³ vg/kg by intravenous administration is appropriate. In certain embodiments, 10¹²-10¹³ rAAV genome copies is effective to target tissues (e.g., the eye). In certain embodiments, 10¹³-10¹⁴ rAAV genome copies is effective to target tissues effective to target tissues (e.g., the eye).

In some embodiments, the rAAV is injected into the subject. In other embodiments, the rAAV is administrated to the subject by topical administration (e.g., an eye drop). In some embodiments, an effective amount of an rAAV is the amount sufficient to express an effective amount of the anti-VEGF agent (e.g., KH902) in the target tissue (e.g., the eyes) of a subject.

In some embodiments, delivery of an effective amount of rAAV by injection (e.g., delivering an rAAV encoding an anti-VEGF agent (e.g., KH902) is in an amount such that it is sufficient to express an effective amount of an anti-VEGF agent (e.g., KH902) in the target tissue). In some embodiments, delivery of an effective amount of an rAAV encoding an anti-VEGF agent (e.g., KH902) is sufficient to deliver 10 μg to 10 mg of an anti-VEGF agent (e.g., KH902) or any intermediate value in between to the subject per eye by suitable routes of administration (e.g., intraocular injection, i.v. injection, intraperitoneal injection and intramuscular injection. In some embodiments, the rAAV encoding an anti-VEGF agent (e.g., KH902) is sufficient to deliver 20 μg to 5 mg or any intermediate value in between of an anti-VEGF agent (e.g., KH902) to the subject per eye. In some embodiments, the rAAV encoding an anti-VEGF agent (e.g., KH902) is sufficient to deliver 10 μg, 20 μg, 30 μg, 40 μg, 50 μg, 60 μg, 70 μg, 80 μg, 90 μg, 100 μg, 200 μg, 300 μg, 400 μg, 500 μg, 600 μg, 700 μg, 800 μg, 900 μg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg or more of an anti-VEGF agent (e.g., KH902) to the subject per eye.

In some embodiments, the rAAV encoding an anti-VEGF agent (e.g., KH902) is administered to the subject once a day, once a week, once every two weeks, once a month, once every 2 months, once every 3 months, once every 6 months, once a year, or once in a lifetime of the subject.

In some embodiments, delivery of an effective amount of rAAV by topical administration such as an eye drop (e.g., delivering an rAAV encoding an anti-VEGF agent (e.g., KH902) is in an amount such that it is sufficient to express an effective amount of an anti-VEGF agent (e.g., KH902) in the target tissue). In some embodiments, the eye drop containing the rAAV encoding is administered to the subject once a week, once a month, once every 3 months, once every 6 months, or once a year.

In some embodiments, the eye drop comprises the rAAV encoding an anti-VEGF agent (e.g., KH902) sufficient to deliver the anti-VEGF agent at a concentration of 1 mg/ml to 20 mg/ml. In some embodiments, the eye drop comprises the rAAV encoding an anti-VEGF agent (e.g., KH902) sufficient to deliver the anti-VEGF agent at a concentration of 2.5 mg/ml to 10 mg/ml. In some embodiments, the eye drop comprises the rAAV encoding an anti-VEGF agent (e.g., KH902) sufficient to deliver the anti-VEGF agent at a concentration of 1 mg/ml, 2 mg/ml, 2.5 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, or 20 mg/ml. In some embodiments, the eye drop is administered at 0.01 ml, 0.02 ml, 0.03 ml, 0.04 ml, 0.05 ml, 0.06 ml, 0.07 ml, 0.08 ml, 0.09 ml, 0.1 ml, 0.2 ml, 0.3 ml, 0.4 ml or 0.5 ml.

An effective amount of rAAVs or composition (e.g., composition containing the isolated nucleic acid or the rAAV described herein) may also depend on the mode of administration. For example, targeting an ocular (e.g., corneal) tissue by intrastromal administration or subcutaneous injection may require different (e.g., higher or lower) doses, in some cases, than targeting an ocular (e.g., corneal) tissue by another method (e.g., systemic administration, topical administration). In some embodiments, intrastromal injection (IS) of rAAV having certain serotypes (e.g., AAV2, AAV5, AAV6, AAV6.2, AAV7, AAV8, AAV9, AAVrh.8, AAVrh.10, AAVrh.39, and AAVrh.43) mediates efficient transduction of ocular (e.g., corneal, retinal, etc.) cells. Thus, in some embodiments, the injection is intrastromal injection (IS). In some embodiments, the injection is topical administration (e.g., topical administration to an eye). In some cases, multiple doses of a rAAV are administered.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf-life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intravitreally, intraocularly, subretinally, intrastromally, subcutaneously, intrapancreatically, intranasally, parenterally, intravenously, intramuscularly, intrathecally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that it is easily syringed. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the anti-VEGF agent described herein (e.g., KH902) is delivered to the subject by ceDNA. Any compositions containing ceDNA encoding the anti-VEGF agent (e.g., KH902) are also within the scope of the present disclosure. In some embodiments, the ceDNA encoding the anti-VEGF agent (e.g., KH902) and the compositions thereof can be administered to the subject using any suitable method described herein. In some embodiments, delivery of an effective amount of the ceDNA encoding the anti-VEGF agent (e.g., KH902) by injection is in an amount such that it is sufficient to express an effective amount of an anti-VEGF agent (e.g., KH902) in the target tissue). In some embodiments, delivery of an effective amount of a ceDNA encoding the anti-VEGF agent (e.g., KH902) is sufficient to deliver 10 μg to 10 mg of an anti-VEGF agent (e.g., KH902) or any intermediate value in between to the subject per eye by suitable routes of administration (e.g., intraocular injection, i.v. injection, intraperitoneal injection and intramuscular injection. In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, Solu-Medrol, Lansoprazole, trimethoprim/sulfamethoxazole, methotrexate, and any combination thereof. In some embodiments, the immunosuppression regimen comprises administering sirolimus, prednisolone, lansoprazole, trimethoprim/sulfamethoxazole, or any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, the methods described in this disclosure further comprise co-administration or prior administration of an agent to a subject administered an rAAV or pharmaceutical composition comprising an rAAV of the disclosure. In some embodiments, the agent is selected from a group consisting of Miglustat, Keppra, Prevacid, Clonazepam, and any combination thereof. In some embodiments, the rAAV (e.g., rAAV for KH902) and the additional agent can be delivered to the subject in any order. In some embodiments, the rAAV (e.g., rAAV for KH902) and the additional agent (e.g., Miglustat, Keppra, Prevacid, Clonazepam) are delivered to the subject simultaneously. In some embodiments, the rAAV (e.g., rAAV for KH902) and the additional agent (e.g., Miglustat, Keppra, Prevacid, Clonazepam) are co-administered to the subject (e.g., in one composition or in different compositions). In some embodiments, the rAAV (e.g., rAAV for KH902) is delivered before the additional agent (e.g., Miglustat, Keppra, Prevacid, Clonazepam). In some embodiments, the rAAV (e.g., rAAV for KH902) is delivered after the additional agent (e.g., Miglustat, Keppra, Prevacid, Clonazepam). In some embodiments, the rAAV (e.g., rAAV for KH902) and the additional agent (e.g., Miglustat, Keppra, Prevacid, Clonazepam) are delivered to the subject at different frequencies, for example, the subject receives the rAAV (e.g., rAAV for KH902) every month, every two-months, every six-months, every year, every two years, every three years, every 5 years, or longer, but receives the additional agent (e.g., Miglustat, Keppra, Prevacid, Clonazepam) daily, weekly, biweekly, monthly, twice a day, three times a day, or twice a week.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

Methods of Treating Diseases Associated with VEGF and/or Angiogenesis

Aspects of the disclosure relate to methods for delivering a transgene encoding an anti-VEGF agent (e.g., KH902) to a subject (e.g., a cell in a subject). In some embodiments, the subject is a human. In some embodiments, the subject is a non-human mammal. Non-limiting examples of non-human mammals are mouse, rat, cat, dog, sheep, rabbit, horse, cow, goat, pig, guinea pig, hamster, chicken, turkey, or a non-human primate.

In some embodiments, the present disclosure relates to a method for inhibiting VEGF activity in a subject in need thereof. In some embodiments, methods described by the disclosure are useful for treating a subject having or suspected of having a disease associated with VEGF. As used herein, “VEGF-associated diseases” refers to set of diseases associated with aberrant VEGF activity/signaling. VEGF is a signal protein produced by cells that stimulates the formation of blood vessels. VEGF is a known factor to induce angiogenesis. In some embodiments, methods described by the disclosure are useful for treating a subject having or suspected of having an angiogenesis associated disease. An angiogenesis associated disease, as used herein, refers to diseases related to abnormal angiogenesis. Non-limiting exemplary angiogenesis associated diseases include angiogenesis-dependent cancer, including, for example, angiogenesis associated eye diseases, solid tumors (e.g., lung cancer, breast cancer, kidney cancer, liver cancer, pancreatic cancer, head and neck cancer, colon cancer, melanoma), blood born tumors such as leukemias, metastatic tumors, benign tumors (e.g., hemangiomas, acoustic neuromas, neurofibromas, trachomas, and pyogenic granulomas), rheumatoid arthritis, psoriasis, rubeosis, Osier-Webber Syndrome, myocardial angiogenesis, plaque neovascularization, telangiectasia, hemophiliac joints, or angiofibroma.

In some embodiments, angiogenesis-associated eye diseases include but are not limited to corneal neovascularization (CoNV), diabetic retinopathy, retinopathy of prematurity, macular degeneration, corneal graft rejection, neovascular glaucoma, and retrolental fibroplasias, epidemic keratoconjunctivitis, Vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren's ulcer, Terrien's marginal degeneration, mariginal keratolysis, rheumatoid arthritis, systemic lupus, polyarteritis, trauma, Wegeners sarcoidosis, Scleritis, Steven's Johnson disease, pemphigoid radial keratotomy, and corneal graft rejection, sickle cell anemia, sarcoid, pseudoxanthoma elasticum, Paget's disease, vein occlusion, artery occlusion, carotid obstructive disease, chronic uveitis/vitritis, mycobacterial infections, Lyme's disease, systemic lupus erythematosus, retinopathy of prematurity, Eales disease, Behcet's disease, infections causing a retinitis or choroiditis, presumed ocular histoplasmosis, Bests disease, myopia, optic pits, Stargardt disease, pars planitis, chronic retinal detachment, hyperviscosity syndromes, toxoplasmosis, trauma or post-laser complications.

As used herein, the term “treating” refers to the application or administration of a composition comprising an anti-VEGF agent (e.g., KH902) to a subject, who has a symptom or a disease associated with aberrant VEGF activity or angiogenesis, or a predisposition toward a disease associated with aberrant VEGF activity or angiogenesis, with the purpose to cure, heal, alleviate, relieve, alter, remedy, ameliorate, improve, or affect the disorder, the symptom of the disease, or the predisposition toward a disease associated with aberrant VEGF activity or angiogenesis. In some embodiments, administration of an anti-VEGF agent results in a reduction of VEGF activity by 2%, 5%, 10%, 20%, 30%, 40%, 50%, 60%, 70%, 80%, 90%, or 100% compared to a reference value. Methods of measuring VEGF activity are known in the art. Non-limiting exemplary reference value can be VEGF activity of the same subject prior to receiving anti-VEGF agent treatment. In some embodiments, administration of an anti-VEGF agent results in a reduction of angiogenesis by 2%, 5%, 10%, 20%, 30%, 40%, 50%, 60%, 70%, 80%, 90%, or 100% compared to a reference value. Methods of measuring angiogenesis are known in the art. Non-limiting exemplary reference value can be level of angiogenesis of the same subject prior to receiving anti-VEGF agent treatment.

In some embodiments, the present disclosure relates to a method for reducing corneal neovascularization (CoNV) in a subject in need thereof (e.g., reducing CoNV relative to a untreated subject, or in the subject prior to the administration). In some embodiments, the method reduces CoNV by at least 5%, at least 10%, at least 20%, at least 30%, at least 40%, at least 50%, at least 60%, at least 70%, at least 80%, at least 85%, at least 90%, at least 95%, at least 99%, or 100% relative to an untreated subject, or in the subject prior to the administration). Methods of measuring CoNV are known in the art (e.g., optical coherence tomography angiography (OCTA), indocyanine green angiography (ICGA), etc). Any suitable method for measure CoNV can be used herein.

Alleviating a disease associated with aberrant VEGF activity or angiogenesis includes delaying the development or progression of the disease, or reducing disease severity. Alleviating the disease does not necessarily require curative results. As used therein, “delaying” the development of a disease (such as a disease associated with aberrant VEGF activity or angiogenesis) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with aberrant VEGF activity or angiogenesis includes initial onset and/or recurrence.

### EXAMPLES

### Example 1: A rAAV Vector Platform to Deliver Conbercept (KH902)

An rAAV vector platform to deliver Conbercept (KH902), an anti-VEGF therapeutic agent into the retina via intravitreal administration (or other routes) was described here. The unique design is a single-strand AAV vector genome that contains the KH902 transgene driven by the CMV enhancer/chicken β-actin promoter regulatory cassette (FIGS. 1A-1C). A Kozak sequence was designed upstream of the KH902 start codon to enhance translation (FIG. 1C). When the cis-plasmid (FIG. 1A) was delivered into packaging cell lines that expressed the AAV Rep and Cap genes and obligatory helper genes via trans-plasmid co-transfections or by stable integration, sequences that include and are flanked by the inverted terminal repeat sequences (ITRs) were packaged into AAV capsid virions.

Cells were infected or transduced by the resulting ssAAV-KH902 virions expressed secreted KH902, which was detected by standard Western blot analysis (FIG. 2). Transduction of retinal pigment epithelial (RPE) cell lines with ssAAV-KH902 packaged into AAV2 capsid, resulted in protein expression of KH902 with similar molecular weights as the Conbercept drug, which was produced in Chinese Hamster Ovary (CHO) cell lines. This data indicates that the rAAV-KH902 vector can be packaged into different AAV capsids and when infected into cells can secrete KH902 effectively.

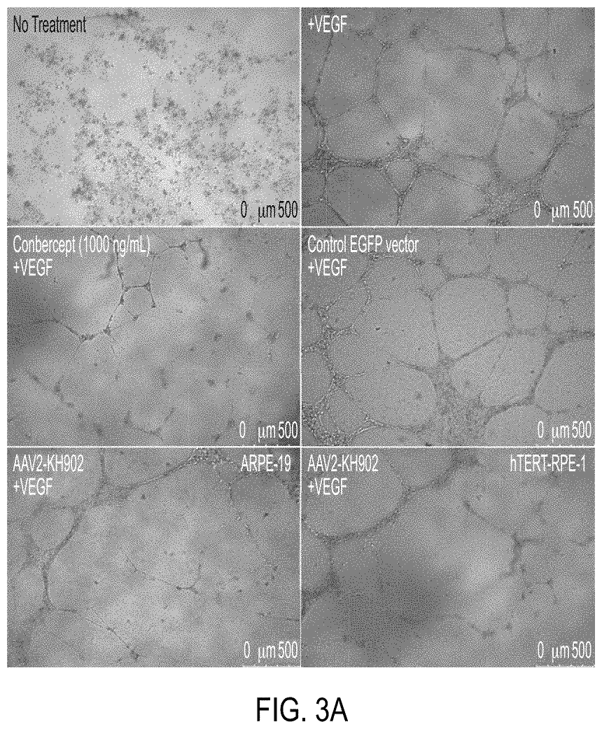

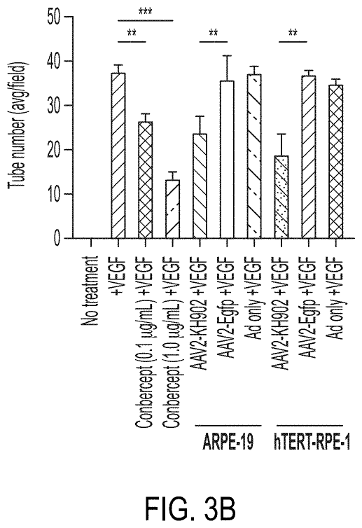

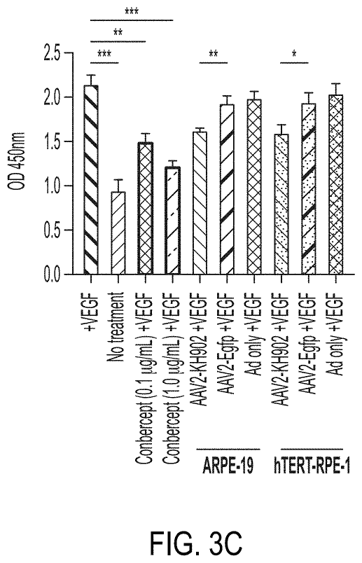

Conditioned media from RPE cells infected with rAAV-KH902 robustly inhibited angiogenesis as indicated by a reduction in vascular endothelial growth factor (VEGF)-induced tubulogenesis (FIGS. 3A and 3B) and proliferation (CCK-8, FIG. 3C) of human umbilical vein endothelial cells (HUVECs) in the same fashion as the Conbercept drug. This data suggest that cells infected with rAAV-KH902 can express and secrete functional anti-angiogenic KH902 in vitro.

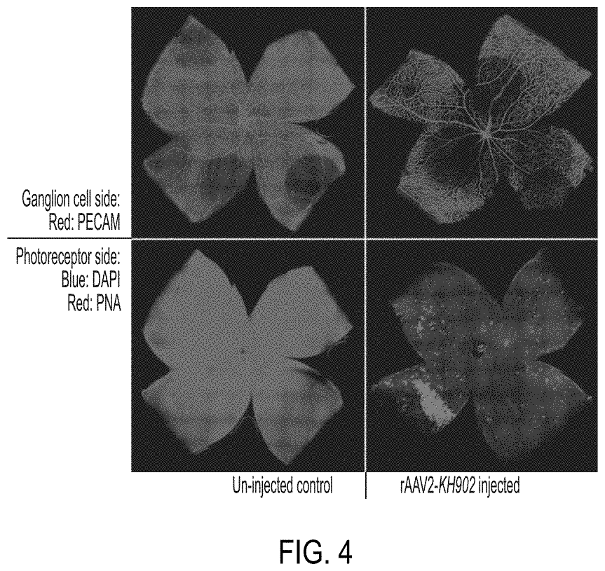

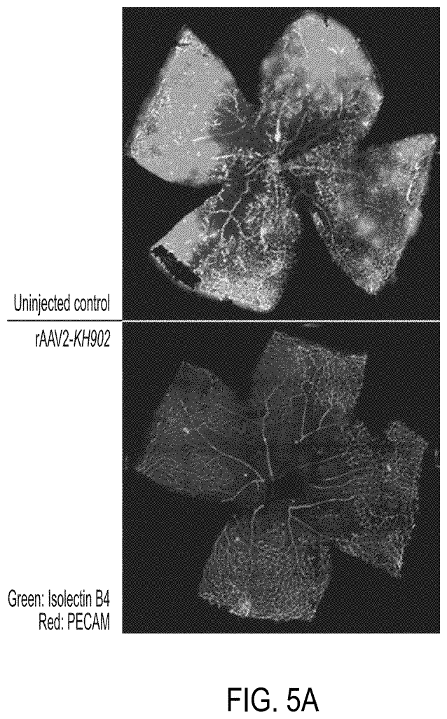

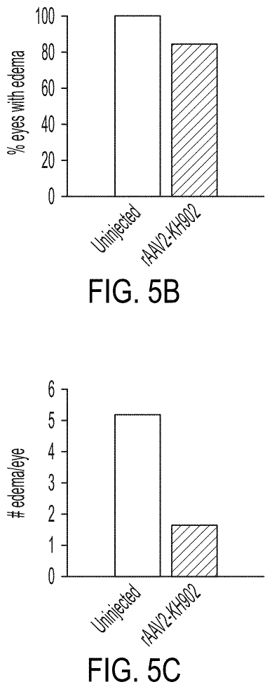

Injection of rAAV2-CBA-KH902 into the vitreous of neonatal mouse pups prevented normal retinal vascular development (FIG. 4). This data suggests that AAV-KH902 virions inhibited vascularization in vivo. To determine whether this vector design can inhibit choroidal neovascularization, the rAAV2-CBA-KH902 vector was tested in a mouse model for retinopathy of prematurity (ROP) (FIG. 5). Intravitreal injection of rAAV2-CBA-KH902 and subsequent hyperoxia treatment of mice led to a reduction in the percentage eyes with detectable edemas and the number of edemas in eyes of treated mice as compared to control uninjected eyes. This data suggests that AAV-KH902 is a potentially viable gene therapy platform for preventing and, possibly reversing, choroidal neovascularization.

### Example 2: Intravitreal Injection of AAV2 Vector is Effective at Delivering KH902 to Prevent Oxygen-Induced Retinopathy and Vascularization in Mice

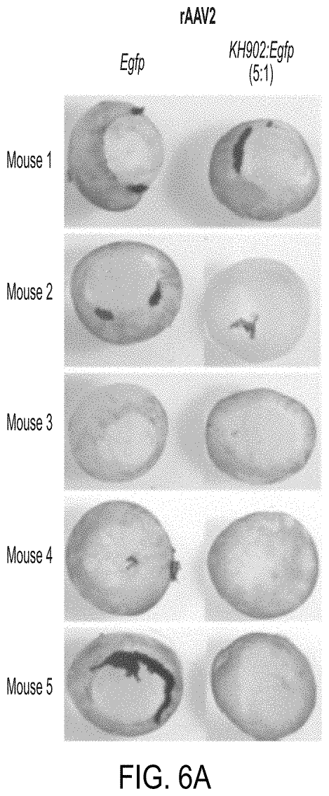

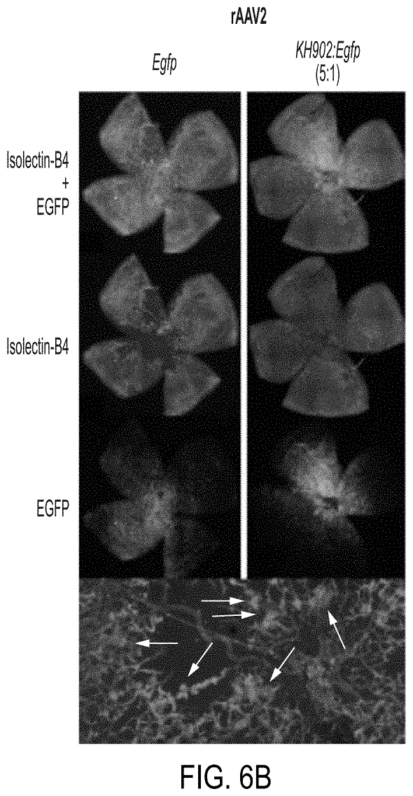

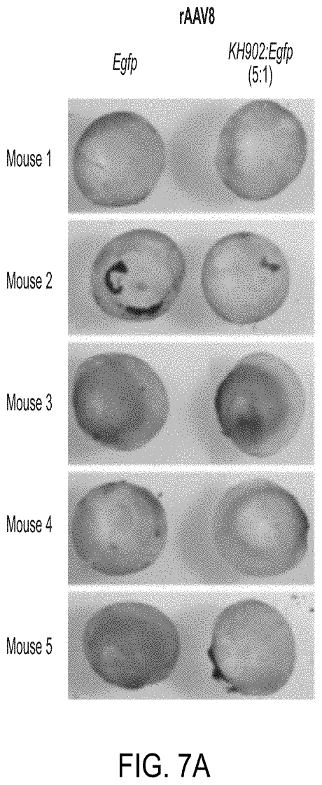

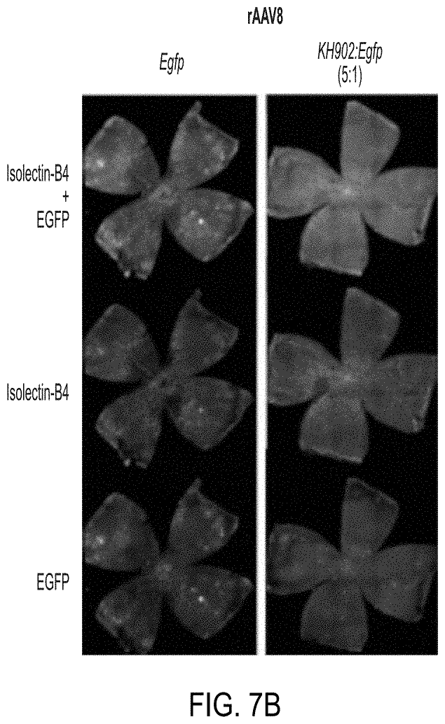

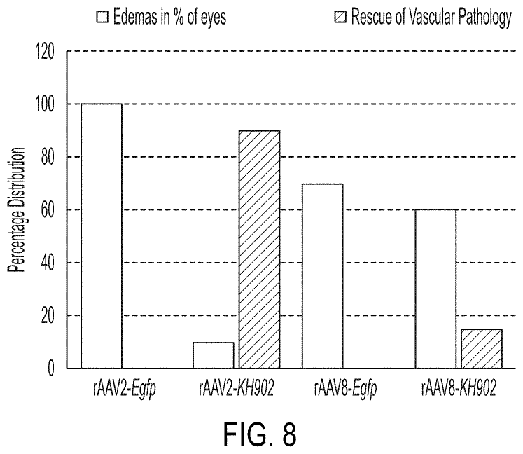

Neonatal mice were treated by intravitreal injection with vector at post-natal days (PN) 1-3. Each mouse was treated in one eye with vector packaging the EGFP transgene (rAAV-EGFP), and the opposing eye with a 5:1 ratio mixture of vector packaging the KH902 transgene (rAAV-KH902) and rAAV-EGFP, respectively. In all cases, the total dose was 1.5E⁹ vg per eye in a 1 μL volume. Mice were then kept at 70% oxygen until PN 7 and placed in normoxic conditions (20-21% oxygen) until PN 11. Mice were sacrificed at PN 18 and eyes were harvested and visualized (FIGS. 6A-6B and FIGS. 7A-7B). The pathology of treated eyes was then scored by visual inspection and scored (FIG. 8). Eyes treated with rAAV-EGFP alone are indicative for the extent of hyperoxia induction and serves as an internal control for variability of pathology. It should be noted that the absence of an edema does not mean that hyperoxia failed to induce retinopathy, nor does the presence of an edema in rAAV-KH902 treated eyes mean that the vector was non-effective. Rescue of vascular pathology is determined by the presence or absence of aneurysm nodules.

In rAAV2-Egfp treated eyes, which serve as negative controls, vascular pathologies were observed as a result of over proliferation and formation of vascular aneurysm nodules (FIG. 6B, bottom panel). Eyes treated with rAAV2-KH902 efficiently prevented the pathologies (FIGS. 6A-6B) and also reduced vascular development to a certain degree (FIG. 6B, right panels). In contrast, rAAV8-KH902 is very inefficient in preventing vascular pathologies (FIG. 7A-7B). This observation correlates with the low transduction of rAAV8-EGFP in retinal tissues (FIG. 7B, left panels). Nonetheless, in areas where there is some transduction, rAAV8-KH902 is able to partially prevent pathologies (FIGS. 7B, right panels and FIG. 8).

Overall, induction of retinopathies by hyperoxia worked in all mice even if the eye did not develop an edema (FIG. 8). Importantly, the KH902 transgene is able to reverse pathological vascularization with rAAV2, but poorly with rAAV8. However, the current results do not predict the outcome of treatments in adult animals, as regular vascular development is completed by then.

### Example 3: Human Derived AAV Capsid Variants for Efficient and Safe Gene Delivery to Retina

To evaluate whether AAV2 variant capsids and AAV2/3 hybrid capsids are capable of delivering a transgene to retina cells, AAV2 variants and AAV2/3 hybrid capsids were packaged into rAAV carrying a barcoded EGFP transgene, and injected into mice either intravitreally or subretinally. The expression of GFP in the retina reflects the capability of a certain capsid protein in transducing cells in the retina.

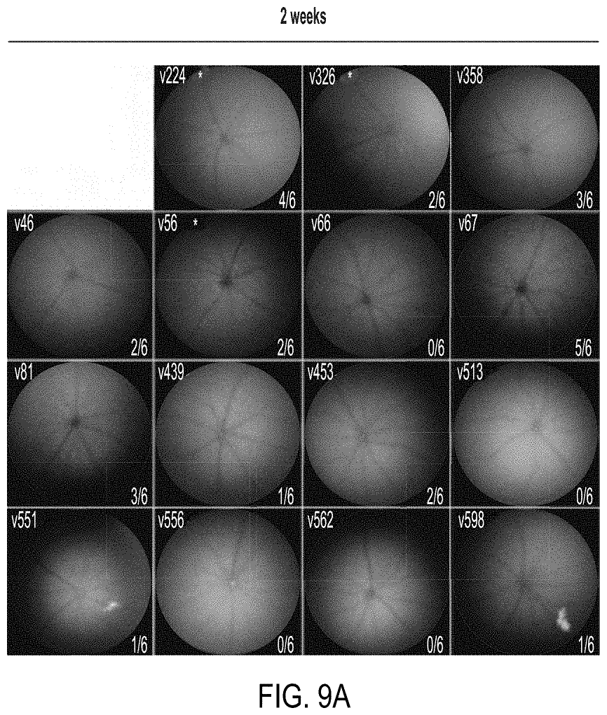

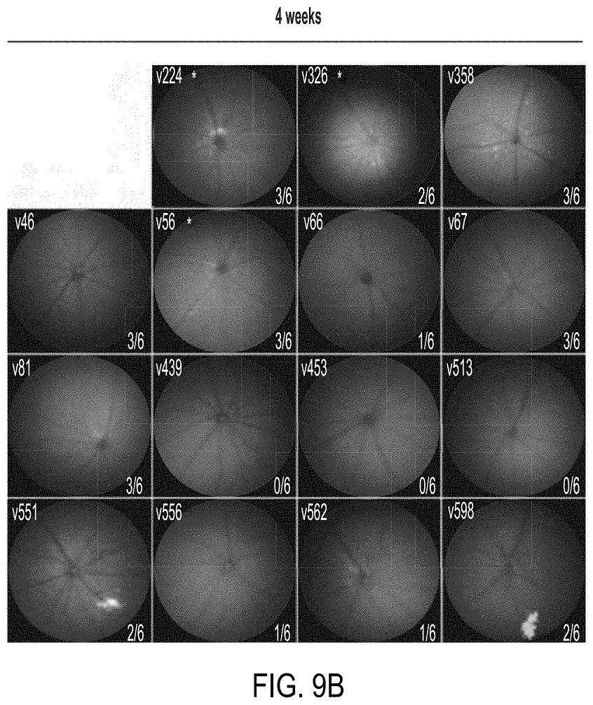

In an initial study, eight AAV2 variants (v224, v326, v358, v46, v56, v66, v67, and v81) and seven AAV2/3 hybrid variants (v439, v453, v513, v551, v556, v562, and v598) were tested. The rAAVs comprising each of the capsid variant and the barcoded EGFP transgene were injected to mice via intravitreal administration (3 mice/group; 45 mice total). Transduction efficacy was observed by fundoscopy two weeks (FIG. 9A) and four weeks (FIG. 9B) after injection. Among all variants, v56, v224, and v326 had the highest transduction as assessed by funduscopy. These three capsids were used to package KH902 for subsequent studies in Laser-induced choroidal neovascularization mouse models. In this study, it was observed that the AAV2 variants performed better than the AAV2/3-hybrid variants in mice.

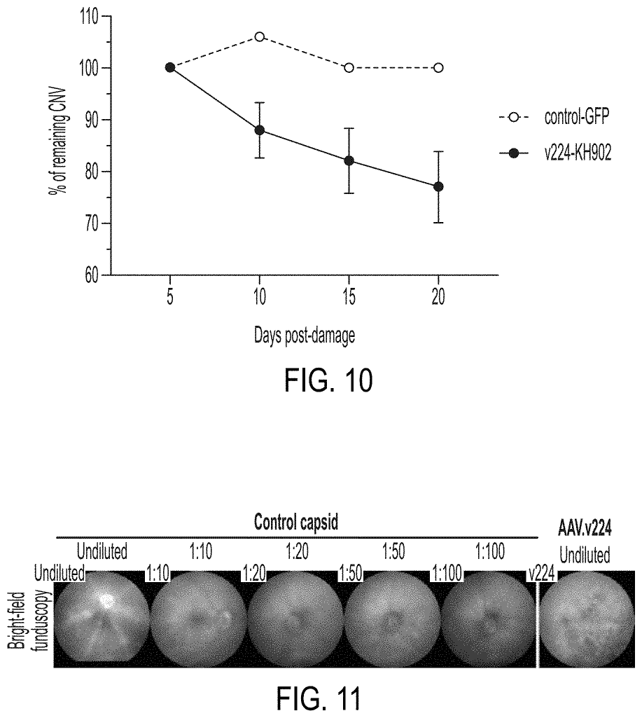

The efficacy of rAAV-KH902 was investigated in a laser-damage treatment model. Laser damage induces Choroidal neovascular (CNV) events, KH902 is capable of reducing CNV in the eyes after laser damage. Both the number of CNV or the size of the CNV can be measured as indicators for efficient delivery of KH902 into the eyes. In the current study, mouse eyes were damaged with laser 5 days prior to rAAV injections. Mice were injected with Control-GFP or AAVv224-KH902. As shown in FIG. 10, Mice treated with AAVv224-KH902 was able to reduce the number CNV to less than 80% 20 days post laser damage as compared to the control-GFP group. AAVv56-KH902 and AAVv326-KH902 showed similar therapeutic efficacy as AAVv224-KH902 in the same mouse model. Further, surface area of CNV is also measured, and it is expected that the delivery of KH902 by AAV2 variant or AAV2/3 hybrid variant is able to reduce the size of CNV.

It was previously observed that overexpression of KH902 causes lesions in the eye in a dose dependent manner due to the accumulation of immune cells in the vasculature. Such lesions can be observed as white streaks in the eye by bright-field funduscopy. To test whether v224-KH902 would cause such lesions in the eye, mice were injected intravitreally with v224-KH902, a control-capsid-KH902 that were previously observed to cause lesions in the eye, 1:10 dilution of the control capsid-KH902, 1:20 dilution of control capsid-KH902, 1:50 dilution of control capsid-KH902, or 1:20 dilution of control capsid-KH902. As shown in FIG. 11, undiluted control capsid-KH902 caused lesions in the eye, and the dilution of control capsid-KH902 reduced the lesions. No lesions were observed in mouse eyes injection with v224-KH902.

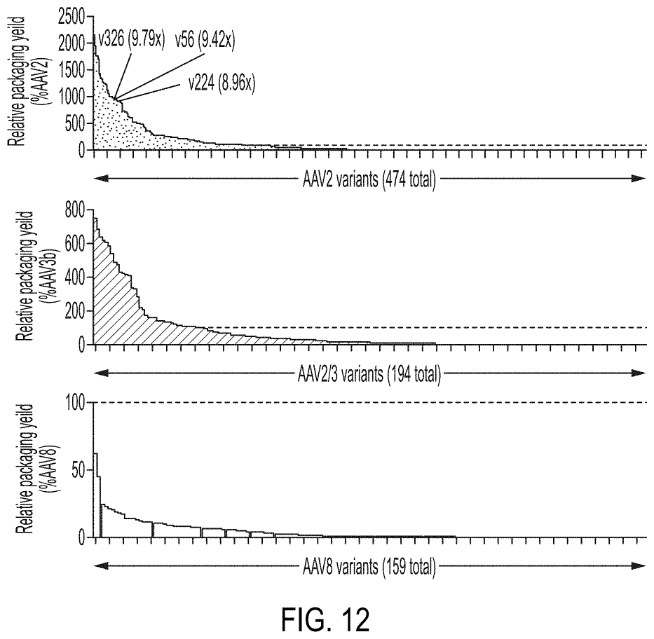

Further, the capsid variants (e.g., AAV2 variants, AAV2/3 hybrid variants and AAV8 variants) showed better packaging efficiency compared to their respective prototypic capsid proteins. As shown in FIG. 12, in vitro packaging yield assessment via crude-lysate PCR was graphed as Waterfall plots, which show the relative packaging yields for AAV2 variants (top panel), AAV2/3 variants (middle panel), and AAV8 variants (bottom panel). The packaging yield values for each capsid are expressed as a percentage of yields conferred by their prototypic forms: AAV2, AAV3b, and AAV8, respectively. Capsid variants v56 showed 9.42 folds increase over AAV2; v224 showed 8.96 folds increase over AAV2, and v326 showed 9.79 folds increase over AAV2. The total number of capsids displayed are shown on the x-axes.

### Example 4

The normal mammalian cornea is transparent and devoid of blood and lymphatic vessels. In pathological conditions where the balance of pro- and anti-angiogenic factors is disrupted, such as inflammation, hypoxia, and limbal barrier dysfunction, corneal neovascularization (CoNV) can occur. CoNV decreases corneal transparency and leads to visual impairment. It affects over 1.4 million people per year in the United States Current treatments for CoNV include topical steroids and non-steroid anti-inflammatory administration, laser cauterization, fine needle diathermy, and amniotic membrane transplantation. However, all above methods have limited efficacies and come with related side effects. Vascular endothelial growth factor (VEGF) plays a critical role in corneal angiogenesis, but none of the aforementioned treatments specific target this key molecule. The successful application of anti-VEGF agents in choroidal neovascularization has led to a surge of interest and testing of these agents for their capacity to manage corneal angiogenesis in animal models and clinical trials. For instance, several early studies demonstrated the potential of bevacizumab in suppressing CoNV through topical, subconjunctival, perilimbal, and intrastromal administration in many animal models and clinical trials. Since then, more anti-VEGF medications, such as ranibizumab and aflibercept, have been developed and applied in corneal neovascularization clinical therapy. However, because of their short half-lives, the limited durability of these VEGF-neutralizing proteins is an evident obstacle to achieve sustainable and efficacious treatment for CoNV. The remarkable advancement of gene therapy technologies has inspired efforts to elevate the durability of anti-VEGF agents by packaging an expression cassette that encodes for a VEGF-neutralizing protein into recombinant adeno-associated virus (rAAV) vectors, which are highly attractive vehicles for the in vivo delivery of therapeutic transgenes in ocular diseases. rAAVs are favorable because of their low immunogenicity, genotoxicity, and high transduction profiles. A single dose of rAAV vector is capable of mediating robust and sustained gene expression, which is important for the goal of achieving therapy and mitigating the treatment burden for patients with chronic corneal diseases.

In this study, the aim was to develop a novel therapeutic approach using rAAV-mediated exogenous KH902 expression with a single dosing to steadily prevent and inhibit angiogenesis in the injured corneas.

Intrastromal Injection of rAAV2 and rAAV8 Vectors Produces Efficient Corneal Cell Transduction

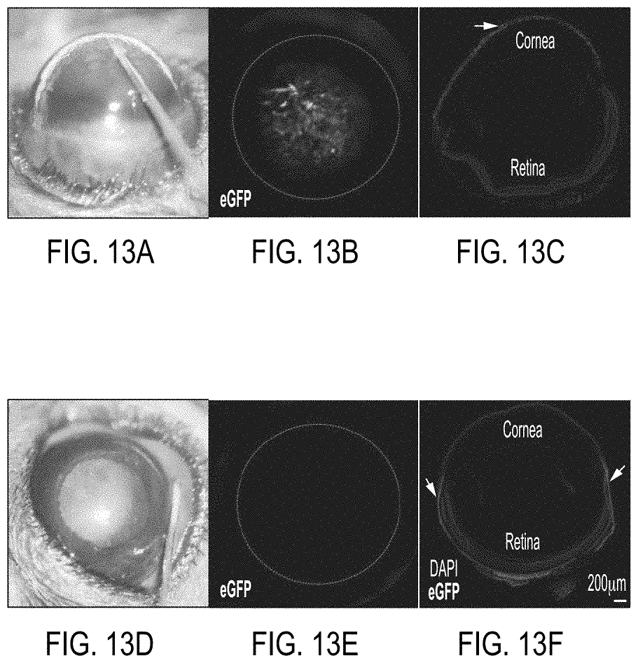

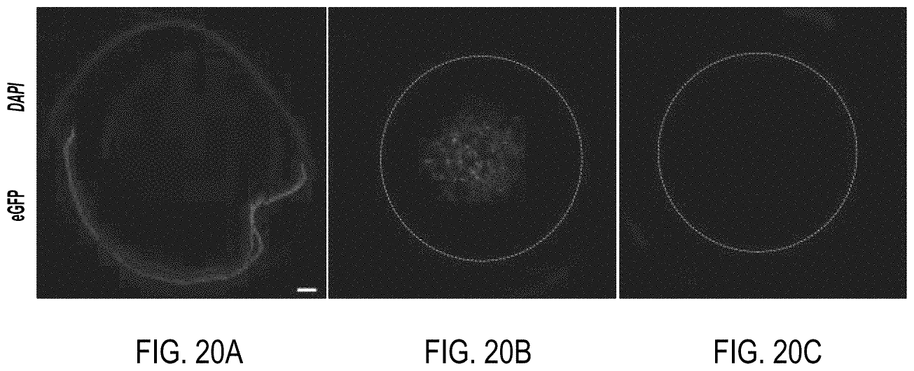

To ensure successful rAAV-mediated gene therapy, the route of application must allow efficient delivery and expression of the therapeutic gene inside the target tissue. Currently, therapeutic agents targeting the cornea are mainly administered via topical instillation, subconjunctival injection, or intrastromal injection. Topical instillation of rAAV vectors without the removal of the superficial epithelium have demonstrated relatively low transduction efficiencies; therefore, the biodistribution of rAAV vectors was primarily compared following subconjunctival and intrastromal injection routes. Mouse cornea were injected with rAAV2 or rAAV8 packaged with the eGFP reporter transgene under a chicken β-actin ubiquitous promoter (rAAV2-eGFP or rAAV8-eGFP) at equal doses (1.6×10¹⁰ genome copies (GCs)/cornea), either via intrastromal or subconjunctival routes (FIGS. 13A, 13D). The level of eGFP expression in the cornea was assessed at two weeks post-administration, through the direct detection of eGFP signal by the live animal imaging system (Micron IV camera). Intriguingly, eGFP signal was successfully detected in the corneas of mice treated by the intrastromal route, but not by the subconjunctival route (FIGS. 13B, 13D, 20B, 20C). To further confirm the eGFP biodistribution pattern in the cornea, eGFP fluorescence was analyzed in enucleated eyeball sections at two weeks post-administration. Consistent with the live imaging data, eGFP was expressed in the entire cornea following intrastromal injection of vector. In contrast, subconjunctival injections led to expression in the limbal, conjunctival, and scleral tissues, while expression in the cornea was poor or non-existent (FIGS. 13C, 13F, 20A). Together, these data demonstrated that intrastromal injection is a more effective route for administering rAAV2 and rAAV8 vectors for widespread transduction in the cornea.

Corneal Cell Tropism and Kinetics of rAAV2- and rAAV8-Mediated eGFP and KH902 Expression

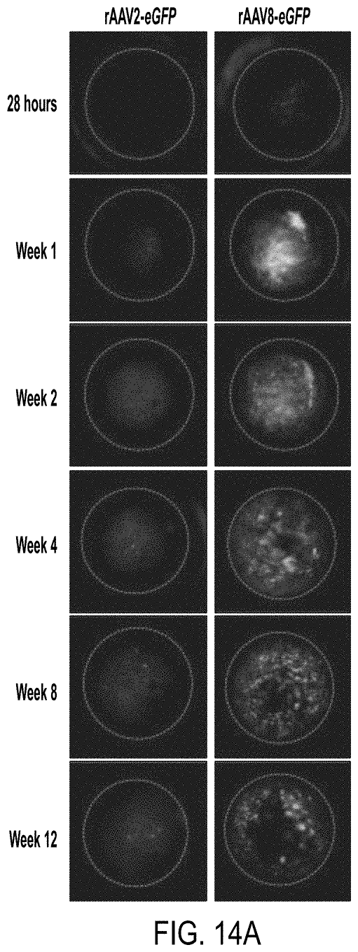

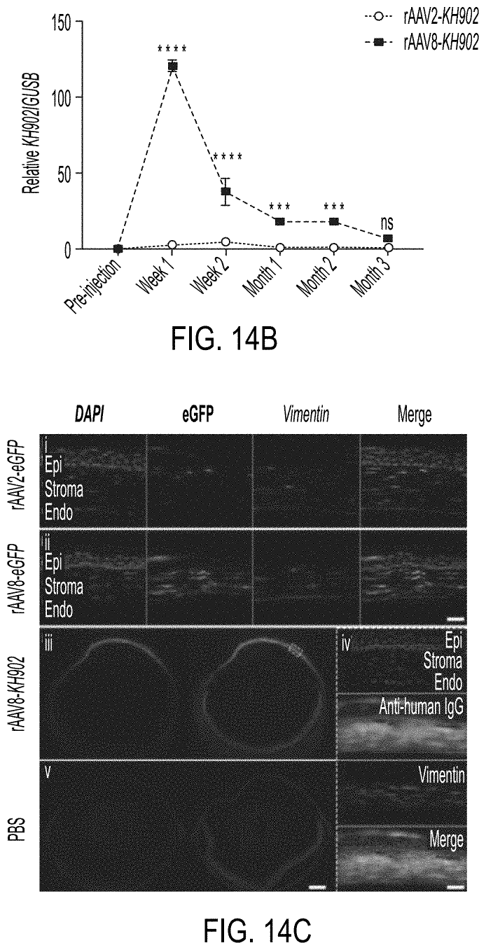

To inspect the kinetics of transgene expression mediated by rAAV2 and rAAV8 via corneal intrastromal injection, rAAV2-eGFP or rAAV8-eGFP was administered intrastromally at an equal dose of 1.6×10¹⁰ GCs per cornea. The eGFP signal mediated by rAAV8 was readily detected as early as 28 hours post-injection by the live animal imaging (FIG. 14A). However, the eGFP signal in rAAV2-eGFP-treated corneas was not observed until one-week post-administration, with a much weaker signal intensity than those conferred by rAAV8-eGFP treatment (FIG. 14A). Next, rAAV2-KH902 or rAAV8-KH902 were injected intrastromally into wild-type mouse corneas at 1.6×10¹⁰ GCs per cornea and evaluated the relative KH902 mRNA expression at weeks 1 and 2, and at months 1, 2, and 3 by droplet digital PCR (ddPCR). Robust expression of KH902 mRNA was detected in the rAAV8-KH902 group, reaching its peak at one-week post-injection. Meanwhile, rAAV2 also led to detectable KH902 expression, but at significantly lower levels and with a lagging peak at two weeks post-injection. After reaching peak levels, KH902 mRNA expression that was mediated by rAAV2 and rAAV8 gradually declined, but remained detectable for up to three months post-injection, the final time point in the experiment (FIG. 14B). Notably, relative KH902 mRNA expression that was mediated by rAAV8 was higher than what was achieved by rAAV2 at every time point. This observation was consistent with eGFP signals conferred by the rAAV-eGFP vectors (FIG. 14A).

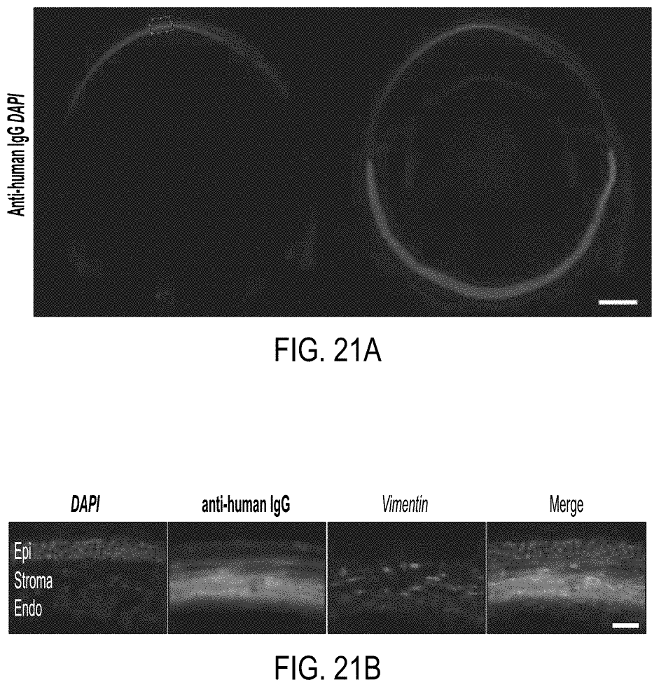

To investigate the tropism of rAAV2 and rAAV8 transduction in the mouse cornea, corneal tissue sections from intrastromal injections with rAAV2-eGFP or rAAV8-eGFP were analyzed at two weeks post-injection (FIG. 14C). Results showed that the eGFP signal mainly co-localized with Vimentin, a keratocyte marker, indicating that rAAV2 and rAAV8 mainly transduced keratocytes. Sporadic expression in epithelial cells was also observed. On the other hand, neither rAAV2-eGFP nor rAAV8-eGFP transduced corneal endothelial cells (FIG. 14C-i, ii). Subsequently, the distribution of KH902 protein was probed using an anti-human IgG (H+L) antibody in the corneas that were transduced with rAAV2-KH902 or rAAV8-KH902 (1.6×10¹⁰ GCs/cornea). KH902 protein following rAAV2 and rAAV8 transduction was primarily found in keratocytes and rarely in corneal epithelial cells (FIG. 14C-iii, iv and FIGS. 21A-21B), which was consistent with the eGFP expression pattern following rAAV2-eGFP and rAAV8-eGFP transduction. However, the results clearly show that KH902 protein distributed not only in cell bodies of keratocytes but also diffused throughout the whole corneal stromal layer. This is attributed to the fact that KH902 contains a secretory signal peptide at the N-terminus, thus facilitating secretion of the KH902 protein from keratocytes into the corneal stromal matrix.

Overall, the data indicated that rAAV8 mediated a stronger and earlier onset of KH902 expression in the cornea following intrastromal delivery than by rAAV2, suggesting that rAAV8-KH902 was superior to rAAV2-KH902 in the treatment of corneal neovascularization. Moreover, the expression of KH902 that was mediated by rAAV8 was sustained for up to three months and dispersed through the entire corneal stromal layer. This finding implicated a potential robust capacity for rAAV8-KH902 to neutralize VEGF, and consequently, to attenuate CoNV.

Characteristics of Immunological Responses to rAAV2-KH902 or rAAV8-KH902 in the Mouse Cornea

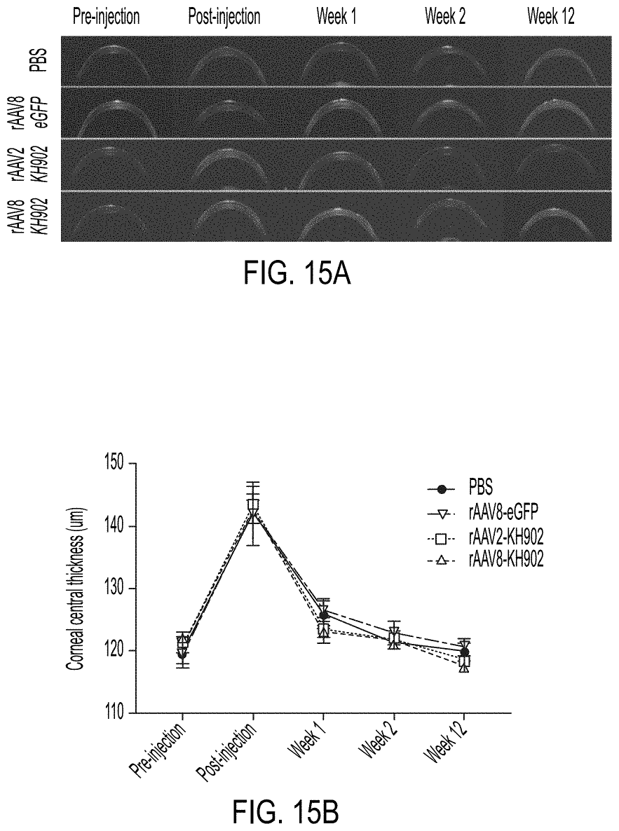

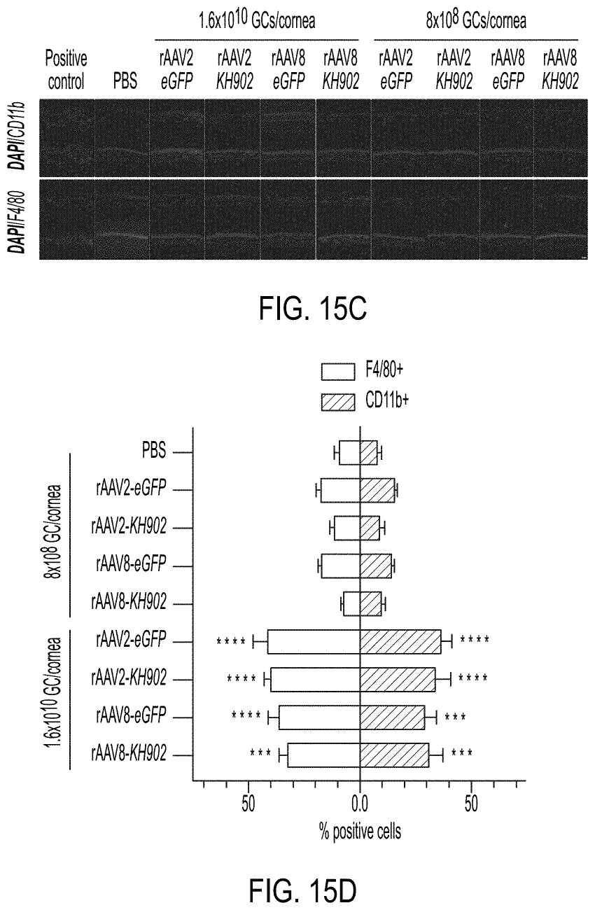

Given that the central corneal thickness (CCT) is an indicator of corneal health and physiological function, CCT was analyzed at various time points by optical coherence tomography (OCT) imaging following intrastromal injection with PBS, rAAV8-eGFP, rAAV2-KH902, and rAAV8-KH902 (1.6×10¹⁰ GCs/cornea). Among the groups, PBS and rAAV8-eGFP were used as the injection control and the vector vehicle control, respectively. Immediately following the injection, it was found that the CCT was increased by 22.73±2.93 μm in the PBS group, 23.50±5.37 μm in the rAAV8-eGFP group, 22.12±3.43 μm in the rAAV2-KH902 group, and 22.75±3.12 μm in the rAAV8-KH902 group, and then gradually reduced back to pre-injection levels at the end of week 12 (final time point of data collection) with normal morphological and anatomical structure under OCT scan (FIGS. 15A, 15B). No significant difference of CCT was observed among all groups. All together, these data indicated that intrastromal injection with rAAV vectors and the KH902 transgene product did not alter the central corneal thickness and disrupt corneal physiological structure to produce local scar or Descemet's membrane detachment after three months. Meanwhile, the immune response was also assessed at two weeks post-injection of rAAV-KH902 by estimating the level of monocyte/macrophage markers (CD11b, F4/80) in the cornea. The levels of CD11b+ or F4/80+ cells were significantly higher after high-dose (1.6×10¹⁰ GCs/cornea) rAAV2-eGFP/KH902 and rAAV8-eGFP/KH902 administration, whereas the percentages of CD11b+ or F4/80+ cells in their low-dose (8×10⁸ GCs/cornea) counterparts were significantly lower in comparison, which is at the similar levels as the PBS control (FIGS. 15C, 15D). Therefore, the low dose (8×10⁸ GCs/cornea) injection scheme for rAAV2-KH902 and rAAV8-KH902 delivery was used in the subsequent in vivo CoNV therapy studies.

Treatment with rAAV8-KH902 Via Intrastromal Administration Effectively Inhibits CoNV in an Alkali-Burn Injury Model

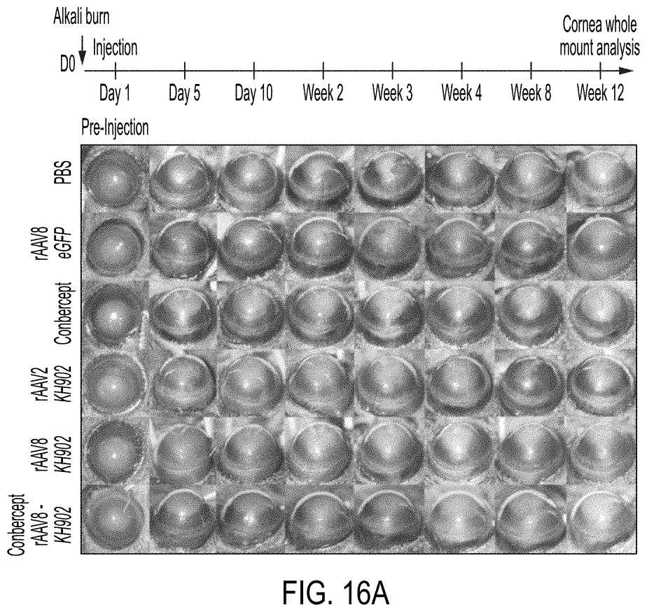

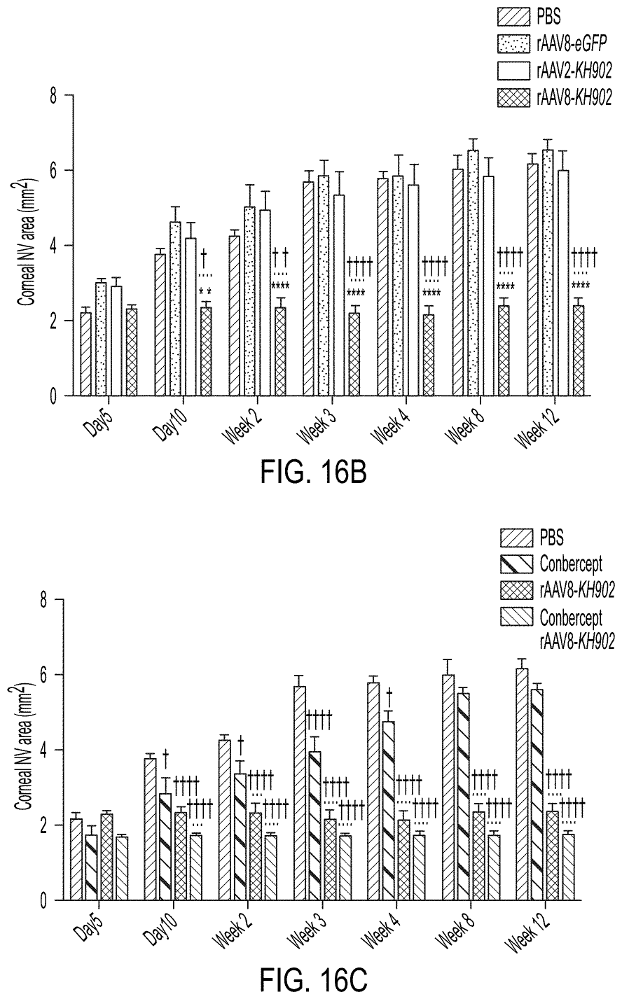

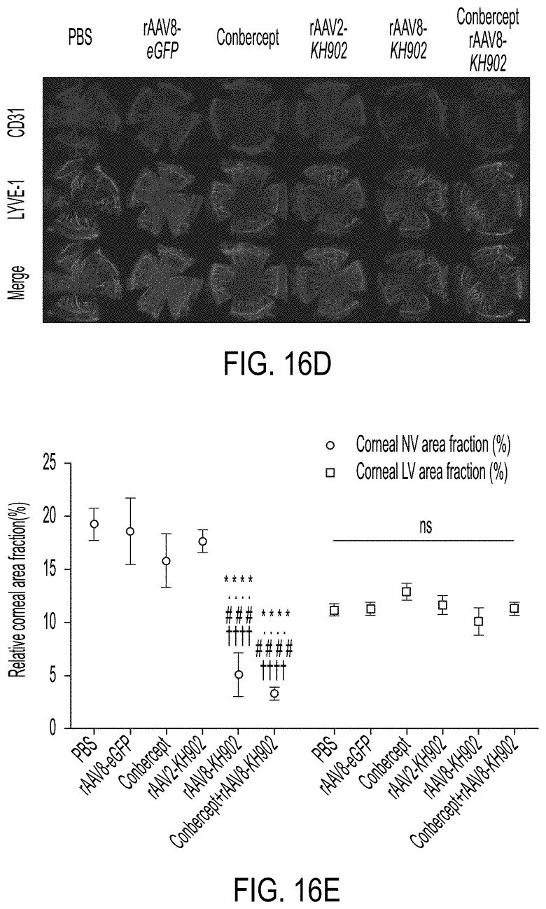

To test whether rAAV2-KH902 and rAAV8-KH902 by intrastromal delivery would therapeutically inhibit corneal neovascularization, alkali burn was applied on mice corneas to create the CoNV model and subsequently injected mice with PBS, rAAV8-eGFP, rAAV2-KH902, or rAAV8-KH902 at the dose of 8×10⁸ GCs/cornea on day one post alkali burn. CoNV progression was tracked at day 5, day 10, as well as 2, 3, 4, 8, and 12 weeks after corneal injury (FIG. 16A). At day 5 post-alkali injury, sprouting and splitting of the CoNV from limbal vascular plexus started to be visible in all groups, with no statistical differences among the groups in the CoNV area. Excitingly, the length and area of CoNV were not evidently increased in rAAV8-KH902 treatment group and remained relatively quiescent from day 5 to week 12 (experimental end point). On the contrary, rAAV2-KH902 failed to inhibit the further growth of CoNV from the limbus to the central cornea at around four weeks post-burn, as did in rAAV8-eGFP and PBS control groups (FIGS. 16A, 16B). To compare the therapeutic efficacy of rAAV8-KH902 and the Conbercept drug, the area of CoNV was quantitively analyzed in both of single-dose Conbercept (10 mg/ml) and rAAV8-KH902 treatment groups. The results showed that there was no significant difference at the early stages of CoNV growth at days 5 and 10. However, in the single-dose Conbercept drug-treated group, CoNV burgeoned from two weeks post-treatment and further progressed to invade the central cornea at four weeks, ending up with intensive CoNV with sizes similar to the PBS control group at twelve weeks. The CoNV area size in the rAAV8-KH902-transduced group was significantly smaller compared to that in the Conbercept drug-treated group from two weeks to twelve weeks post-injection, indicating that rAAV8-KH902 exhibited prolonged anti-VEGF efficacy. In addition, rAAV8-KH902 in combination with Conbercept did not further inhibit CoNV area compared to rAAV8-KH902 alone throughout the observation period (FIGS. 16A, 16C), indicating at this dose, the expression of KH902 that was delivered by rAAV8 was adequate to neutralize VEGF in a timely manner to achieve anti-angiogenic effects. Additional Conbercept drug supplement did not help to further strengthen the therapeutic effect of rAAV8-KH902. For verification, immunostaining data using anti-CD31 (also known as platelet— endothelial cell adhesion molecule 1, PECAM-1) for outlining the blood vessel on corneal whole mounts showed that the CoNV size correlated with the results of the gross vascular pathologies at twelve weeks (FIG. 16D, left panels).

Lymphangiogenesis has also been implicated in the pathological process of CoNV. The formation of new lymphatic vessels from the corneal limbus is believed to be similar to vascular growth during angiogenesis and is mainly induced by the binding of VEGF-C and VEGF-D to VEGFR-2 and VEGFR-3. Given VEGF-A has also been shown to contribute to lymphangiogenesis and Conbercept blocks all VEGF-A isoforms, the effect of rAAV8-KH902 on lymphangiogenesis was also evaluated. Since pathologic lymphatic vessels that invaded into cornea is not directly visible, the mice corneas were collected at week 12 in each group. Corneal whole mounts were double-stained with CD31 as a pan-endothelial marker and LYVE-1 (Lymphatic Vessel Endothelial Receptor 1) as specific lymphatic vessel marker. The area covered by CD31⁺⁺⁺/LYVE-1⁻ blood vessels and CD31⁺/LYVE-1⁺⁺⁺ lymph vessels were measured in cornea whole mounts. In all the experimental groups at the twelve-week follow-up, treatment with rAAV8-KH902 alone and in combination with the Conbercept drug did not reduce the size of lymphangiogenesis, and no significant differences were observed between the PBS control and various treatment groups (FIGS. 16D, 16E).

Dll4/Notch Signaling and ERK Activation are Downregulated by rAAV8-KH902

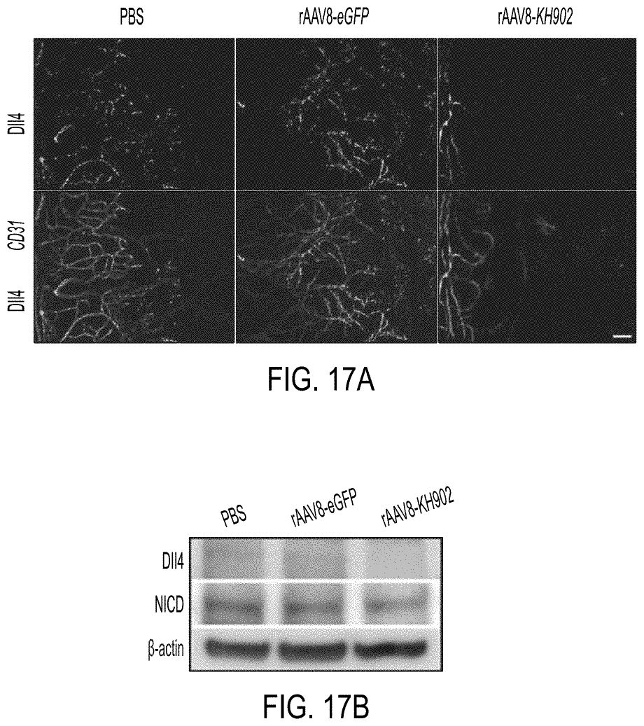

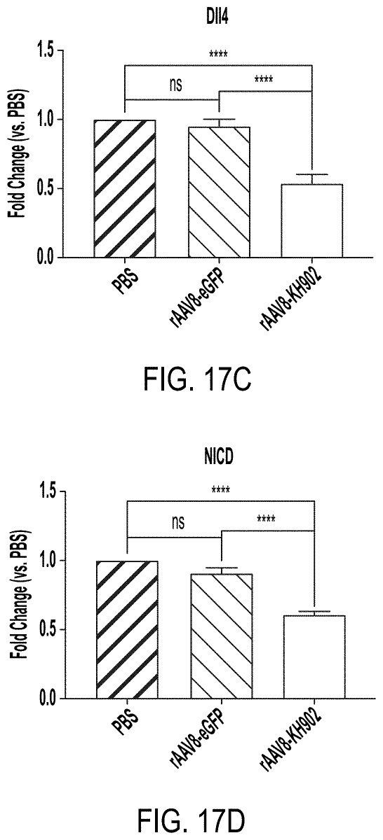

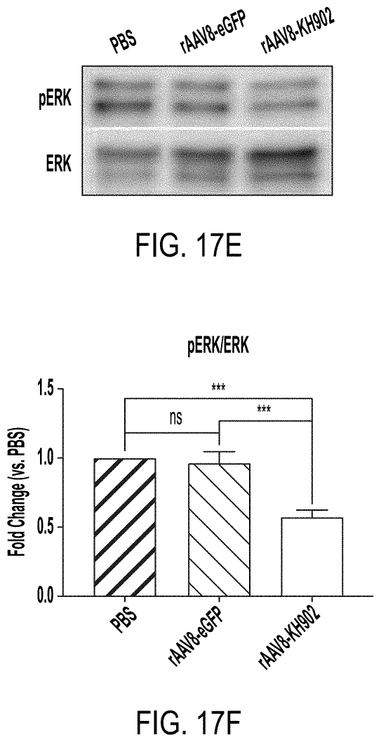

Sprouting angiogenesis is led by endothelial “tip” cells, directing the sprouting process, while endothelial “stalk” cells elongate the neovessel sprouts. During this process, VEGF and Notch signaling pathways are implicated in the selection of tip and stalk cells in the vascular endothelium. Specialized endothelial tip cells lead the outgrowth of blood-vessel sprouts towards the VEGF-A gradient. D114 and reporters of Notch signaling are distributed in a mosaic pattern among endothelial cells of actively sprouting vessels. Under VEGF stimulation, quiescent endothelial cells are induced to form the tip cell filopodia and upregulate the level of D114 expression in the tip cells. In turn, D114 ligand activates Notch signaling in the stalk cells, leading to the release of the active Notch intercellular domain (NICD) from the cell membrane, consequently enabling adequately spaced branching and sprouting. However, previous studies have not addressed the role of D114/Notch signaling in CoNV. Therefore, D114/Notch signaling expression was evaluated in mouse cornea with vigorously growing vessels by immunostaining and Western blot analyses at two weeks post-alkali burn. In PBS and rAAV8-eGFP control groups, D114 was broadly expressed in the corneal neovessel sproutings, suggesting an involvement of D114 in the process of corneal angiogenesis. By contrast, in the rAAV8-KH902 treated group, D114 was rarely detected and the tip cell filopodia were completely retracted (FIG. 17A). These results demonstrated that VEGF-A stimulation was blocked by rAAV8-KH902, thus preventing tip cell migration and CoNV progression. The findings were confirmed by Western blot results, which showed significant downregulation of D114 and NICD expression in the rAAV8-KH902 treated group compared to control groups (FIGS. 17B, 17C, 17D).

VEGF binding to VEGFR results in phosphorylated VEGFR2, initiating downstream signaling pathways relevant to angiogenesis and producing several cellular responses in epithelial cells (ECs). Among these pathways, VEGF-induced ERK1/2 signaling has been extensively studied and is shown to regulate microvascular endothelial differentiation and proliferation. Therefore, mouse corneas were collected at eight days post-alkali burn to assess the level of ERK activation in each condition. The ratio of phosphorylated ERK (pERK) to total ERK (pERK/ERK) was significantly decreased in the rAAV8-KH902 treated group compared to the PBS group and the rAAV8-eGFP treated group by Western blot analysis (FIGS. 17E, 17F), suggesting that blocking VEGF by rAAV8-KH902 resulted in the inhibition of ERK activation in alkali burn-induced CoNV mice.

rAAV8-KH902 Prevents Progression of Pre-Existing Neovascularization in Both Alkali-Burn and Suture Induced CoNV Models

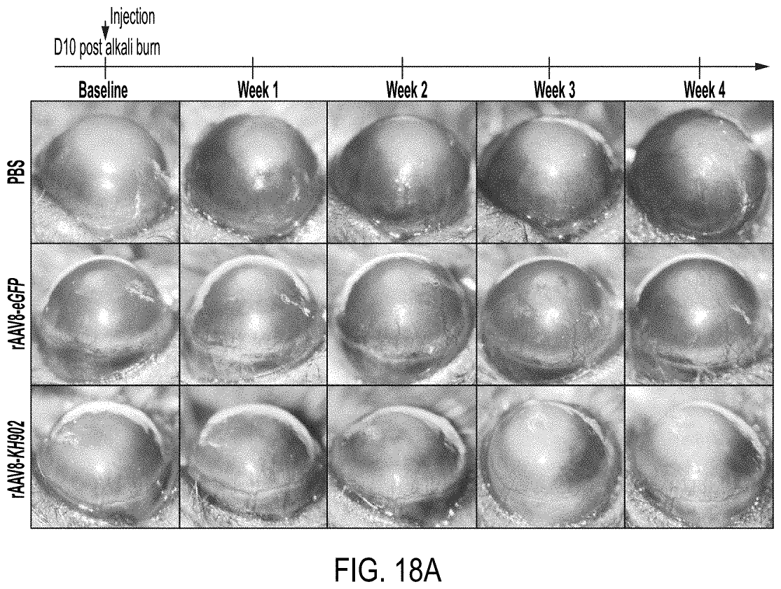

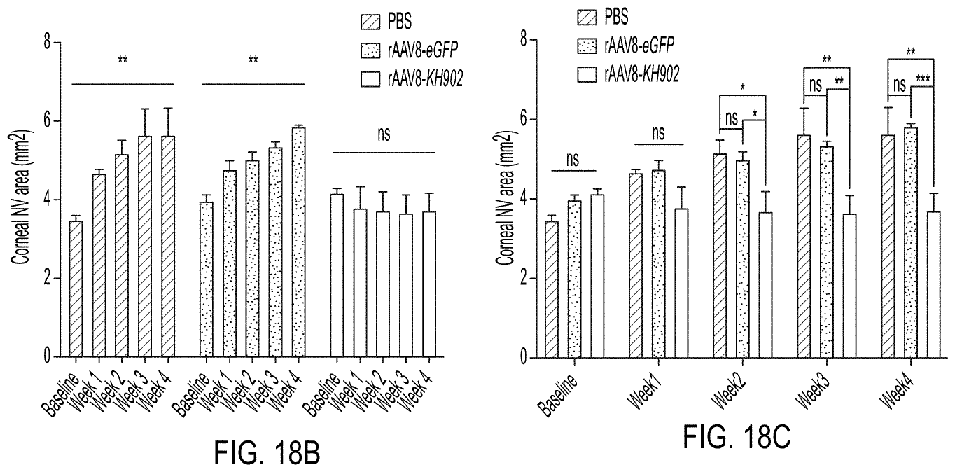

Chemical burn is an acute ocular injury and a complex condition with varied severity and offending lesions. For cases that fail to acquire immediate or intensive management at initial stage, the CoNV is prone to progress rapidly during active stage. It is thus of great interest to explore if rAAV8-KH902 is capable to suppress or even regress the actively expanding CoNV triggered by alkali burn. Mouse cornea was injected intrastromally with PBS, rAAV8-eGFP, or rAAV8-KH902 (8×10⁸ GCs/cornea) at ten days after alkali burn, at which time, CoNV had already invaded into the cornea to varying degrees that is in active stage and continually to grow (FIG. 18A). Once CoNV was induced by alkali burn, no remarkable regression was found in any of the experimental groups through the self-controlled study method (FIGS. 18A, 18B). However, in the rAAV8-KH902 treatment group, the existing CoNV was significantly suppressed and maintained at the pre-treatment state during the four-week follow-up (FIGS. 18A, 18C).

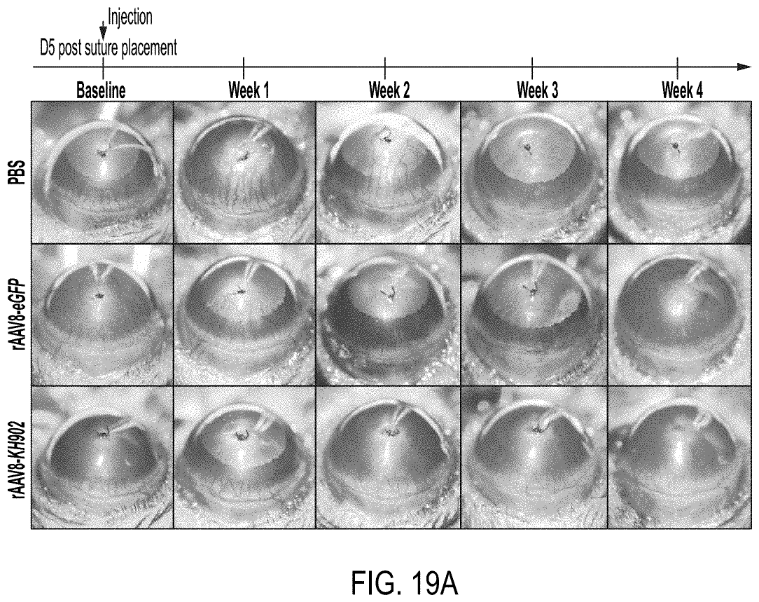

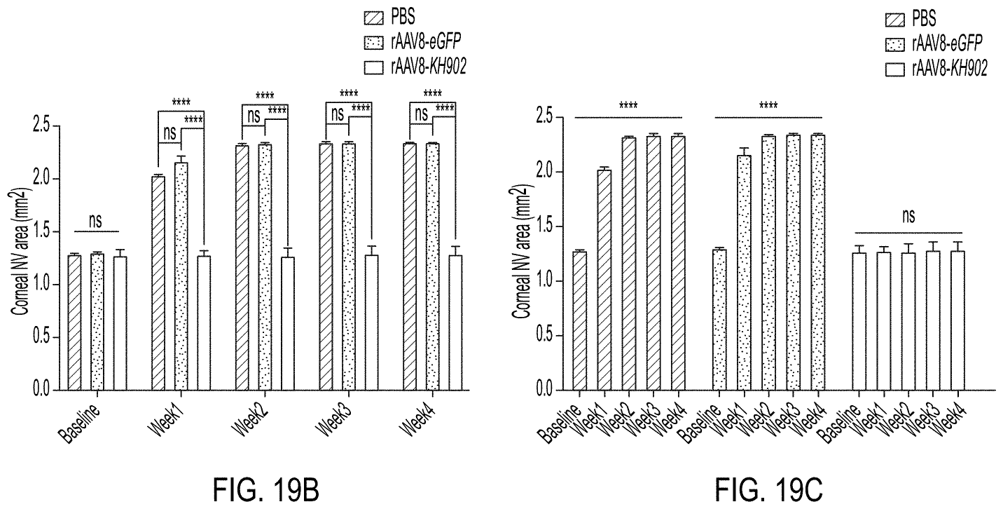

To confirm the effect of rAAV8-KH902 at inhibiting CoNV in a different injury model, it was further explored using the suture-induced CoNV mouse model by placing an intrastromal suture with a knot on the mouse cornea. After 5 days of suture stimulation, CoNV was actively growing and had invaded into the cornea. At this time, mice were injected with PBS, rAAV8-eGFP, or rAAV8-KH902 intrastromally at a dose of 8×10⁸ GCs per cornea. The level of CoNV progression was tracked and quantified before and after injection. Compared with the PBS and the rAAV8-eGFP controls, the progression of CoNV was significantly inhibited with rAAV8-KH902 treatment and the inhibitory effect was sustained to the final timepoint (FIGS. 19A, 19B). Nevertheless, no regression of the established cornea vessels was observed following rAAV8-KH902 treatment (FIGS. 19A, 19C). Therefore, the data confirmed that rAAV8-KH902 had sustained therapeutic effect on existent CoNV in the active stage.

Corneal neovascularization severely affects visual function and can be a pathological sequel of multiple etiologies, such as contact lens wear, dry eye, trauma, chemical burn, limbal stem cell deficiency, ocular surface inflammation and corneal infections with bacteria, fungus and virus. Current therapies are limited by efficacy and safety concerns. Intrastromal injection of Conbercept can inhibit cornea neovascularization but it requires repeated dosing and produces injection-associated side-effects. To reduce the frequency of drug administration, the use of rAAV vectors to mediate KH902 expression in the cornea was explored. It was demonstrated that rAAV8-KH902 generated robust and sustained expression of KH902 in the cornea and successfully inhibited CoNV with a one-time low-dose intrastromal injection without notable side effects and the treatment with rAAV8-KH902 alone was sufficient to suppress angiogenesis at the onset of CoNV in a timely manner.

The window of anti-angiogenic treatment of CoNV is difficult to determine, since different cases have distinct pathological etiologies. The pattern of angiogenesis and the proper therapeutic course strongly depend on the characteristics of the types of preceding stimuli and the underlying pathologies. For instance, in herpes simplex virus (HSV)-induced keratitis, CoNV can be evident as early as day one and may continue to up to three weeks after corneal HSV-1 infection. As the disease progresses, infection, inflammation, and CoNV will trigger each other in a positive-feedback loop, leading to an extended course. In other cases, patients with severe chemical injuries could enter a chronic phase that may persist for more than six weeks, developing significant limbal stem cell deficiency and complications with neovascularization. Further, the data showed that a single dose of rAAV8-KH902 delivery offered at least a three-month therapeutic window, while direct Conbercept application can only last for 10-14 days. Therefore, rAAV8-KH902 continually confers an anti-VEGF effect that significantly prolongs the therapeutic window. This makes a significant difference in reducing the need for repeated dosing of an anti-VEGF drug in patients with chronic corneal diseases.

Angiogenesis is the formation of new vessels from pre-existing blood vessels. It is not only dependent on endothelial cell (EC) proliferation and invasion, but also requires subsequent pericyte coverage for vascular stabilization and maturation. In the absence of pericytes, newly formed ECs are unstable and prone to regression without VEGF stimulation, suggesting immature vessels depend on VEGF for survival and growing. A Study on anti-VEGFs monotherapy showed mature vessels were not effectively induced to regress due to the fact that these vessels less depend on VEGF (49). Likewise, the successful inhibition and failing regression of CoNV in the study also proved that VEGF is important to sustain and prompt newly formed vessels, but is not required to maintain mature blood vessels. Therefore, the earlier rAAV8-KH902 treatment for CoNV would achieve better outcomes.

The route of administration is a critical factor to consider in developing gene therapies for ocular diseases. Various routes of administration are being explored for rAAV gene transduction in cornea tissues. Topical application is the easiest route of administration, but is not ideal for rAAV vectors since they have a relatively low transduction efficiency and there may be potential adverse effects caused by the transduction of non-target tissues when vector is spread through tears. The biodistribution of the cornea was compared between sub-conjunctival and intrastromal injections of rAAV2-eGFP and rAAV8-eGFP. The evidence revealed that intrastromal delivery of rAAV2 or rAAV8 vectors generated more efficient and widespread transduction in the cornea compared to sub-conjunctival injection. In addition, intrastromal injection of rAAV2 and rAAV8 had similar corneal cell tropisms, mainly to keratocytes, with interspersion in epithelial cells, but not endothelial cells. rAAV8-mediated gene expression occurred with an earlier onset and with higher efficiency compared to rAAV2. This explains why rAAV8-KH902 successfully inhibited CoNV, but rAAV2-KH902 failed.

The corneal wound-healing cascade is comprised of angiogenesis, epithelization, and the abnormal deposition of various types of collagens that contribute to corneal scar and opacity. Intrastromal injection of rAAV-KH902 or rAAV-eGFP into healthy corneas did not induce scarring or opacity. This indicated that corneal injury caused by alkali burn is the reason for scarring and lower transparency during the process of wound healing.

In summary, a single low-dose rAAV8-KH902 injection into the corneal stroma led to efficacious inhibition of CoNV for an extended period of time. This study demonstrates the potential long-acting and relative safety of rAAV-based, anti-VEGF gene therapy for CoNV.

Methods

Vector Production

The vectors were packaged with transgene cassettes encoding eGFP or KH902 under the control of a chicken β-actin/cytomegalovirus (CMV) promoter. The vector encoding KH902 was designed with a rabbit globin poly A. Vectors were produced using triple transfection as described. Vectors were purified by CsCl gradient ultracentrifugation and titered by both ddPCR and silver staining of sodium dodecyl sulfate (SDS)—polyacrylamide gels.

Animals

C57BL/6J mice were obtained from Jackson Laboratories (Bar Harbor, Me.), bred and maintained in standardized conditions with a 12 h light/12 h dark cycle in the Animal Facility at the University of Massachusetts Medical School. All experiments were approved by the Institutional Animal Care and Use Committees and in line with ARVO's statement regarding the use of animals in ophthalmology and vision research.

Alkali Burn-Induced CoNV

The mouse model for alkali burn-induced CoNV was performed as previously described (25), with some modifications. Mice were anesthetized via an intraperitoneal injection of ketamine (5 mg/mL) and xylazine (2 mg/ml) combination (10 mL/kg body weight), and the topical anesthetic proparacaine (0.5%) was applied on the corneal surface. Circular filter-paper discs (2-mm diameter) were pre-soaked in 1 M NaOH for 20 s and then placed on the central cornea for approximately 40 s, followed by washing generously with 15 mL sterile saline solution for 1 min.

Suture-Induced CoNV

A modification from a previously described suture technique was performed to induce CoNV (55). Briefly, the right eye of mice received corneal suture placement under general anesthesia [intraperitoneal ketamine (5 mg/mL) and xylazine (2 mg/mL) combination (10 mL/kg body weight)], supplemented by topical anesthesia (0.5% proparacaine). A single 10-0 nylon suture was placed intrastromally with a knot in the temporal cornea of the eye at 1 mm away from the limbus. To ensure the consistency and reproducibility of the procedure, the whole process was performed on each animal by the same researcher under a dissecting microscope.

rAAV Vector Delivery by Intrastromal or Subconjunctival Injection

Intrastromal injections were performed using a previously published method (7). In brief, an incision around 1.0 mm in size was first made in the corneal epithelium equidistance between the temporal limbus and the center of the cornea with the tip of a 30-gauge needle. Then 1.6×10¹⁰ or 8×10⁸ GCs of rAAV vectors in 4 μL of PBS were injected through the incision into the corneal stroma by using a 5-uL Hamilton syringe with a 34-gauge needle (Hamilton, Reno, Nev., USA; 30° bevel angle). Subconjunctival injection was also performed by using a 5-uL Hamilton syringe. A total of 1.6×10¹⁰ GCs of rAAV vectors were injected into the upper, lower, nasal, and temporal sub-conjunctiva, respectively, with 1 μL (0.4×10¹⁰ GCs) injection per each site. Antibiotic ointment was applied after the injections.

In Vivo Fluorescence Imaging

Animals in each group were observed weekly for a total of twelve weeks after rAAV administration. eGFP expression in the mouse eye was captured by a Micron IV camera (Phoenix Research Labs, Pleasanton, Calif.).

Corneal Optical Coherence Tomography

Mice corneal optical coherence tomography (OCT) was performed using a Micron IV OCT Imaging system (Phoenix Research Labs, Pleasanton, Calif.). Mice were anesthetized via an intraperitoneal injection as aforementioned. The mouse cornea was placed towards the lens of camera and the entire anterior chamber was imaged. Corneal central thickness (CCT) was then measured in captured images by ImageJ software (available online at imagej.nih.gov/ij/). CCTs were measured before injection, immediately after injection, and at 1-, 2- and 12-weeks post-injection.

Anterior Color Imaging and Quantification Analysis of Corneal NV

Procedures were performed under general anesthesia and topical eyedrops as mentioned before. The mouse eye was placed under the ophthalmic surgical microscope (WILD HEERBRUGG) and the cornea from different angles were imaged by a digital camera attached to the microscope. CoNV was analyzed at the set time points using ImageJ software. The area of CoNV was calculated by using the following formula: Area (mm2)=CN/12×3.1416×[R²−(R−VL)²], where CN is the clock-hours of NV; R is the radius of the cornea; and VL is the longest vessel length, extending from the limbal vasculature (56). Color images of each cornea in a live mouse were performed in eight different angles and the area of corneal NV was calculated four times at each angle accordingly.

Immunohistochemistry of Whole Cornea Flat Mounts

Eyeballs were enucleated and fixed with 4% PFA for 1 hour at room temperature after a small hole was made at the limbus with a needle. The excised eyeballs were then prepared for whole-mount staining with a modification to previous reports (31). In brief, the cornea and sclera were separated by the incision along the limbus, followed by removal of the lens and iris. Four radial cuts in the cornea were made to allow whole-mount flattening. Then the tissues were washed by 0.3% Triton X-100 in PBS and blocked with blocking buffer (0.3% Triton X-100/5% normal bovine serum albumin (BSA, Cell Signaling Technology)/1×PBS for 1 hour. The corneas were stained overnight at 4° C. with rat anti-CD31 (PECAM-1, 1:400, sc-18916, Santa Cruz, Santa Cruz), rabbit anti-mouse LYVE-1 (1:200, 11-034, AngioBio Co), or goat anti-mouse D114 (1:40, AF1389, R&D Systems). The primary antibodies were then detected with goat anti-rabbit, anti-rat, or donkey anti-goat secondary antibodies conjugated with Alexa flour 488 or 594 (Thermo Fisher Scientific, Singapore). After the final wash with 0.3% Triton X-100 in PBS, the corneal tissues were mounted endothelial side down and imaged by a Leica DM6 microscope with a 16-bit monochrome camera. Image processing was performed with Adobe Photoshop CC 2019 to improve definition. Areas covered by the markers of blood and lymph vessels were detected and measured using ImageJ software. Entire corneas were analyzed by two independent observers, blind to treatment status to minimize sampling bias.

Histology and Immunohistochemistry of Cornea Cryosections

The freshly excised eyeballs were directly embedded in O.C.T. (Fisher Scientific, Pittsburgh, Pa.) in preparation for sectioning. 14 μm-thick cryosections were made from frozen blocks (Leica CM3050 S, Leica Biosystems Inc., Buffalo Grove, Ill.). Following the fixation of sections with 4% PFA for 15 min at room temperature, tissue sections were rinsed by 0.3% Triton X-100 in PBS and blocked with blocking buffer (1×PBS/1% BSA/0.3% Triton™ X-100) for 1 hour. Slides were stained overnight at 4° C. with primary antibodies. The primary antibodies used were: rat anti-F4/80 (1:400, NB600-404, Novus), rat anti-mouse CD11b (1:50, #550282, BD Pharmingen), rabbit anti-Vimentin (1:100, #5741, Cell Signaling Technology), and donkey anti-human IgG (H+L) conjugated with Alexa Fluor 488 (1:400, #144222, Jackson ImmunoResearch Laboratories Inc.), which were all diluted in PBS with 0.3% Triton X-100 and 5% BSA. The secondary antibodies with DAPI (#9542, Sigma-Aldrich) counterstained used were goat anti-rat IgG-Alexa Fluor 594 and goat anti-rabbit IgG-Alexa Fluor 594. Fluorescence images were acquired by a Leica DM6 microscope. Image analysis was performed with Adobe Photoshop software. CD11b+ or F4/80+ cells were detected and counted by using ImageJ software.

KH902 mRNA Expression Analyses

RNA from normal mouse corneas treated or untreated with rAAV2-KH902 or rAAV8-KH902(4 corneas/group) were isolated at weeks 1 and 2 and months 1, 2, and 3 post-treatment using the RNeasy Plus Micro Kit and reverse transcribed into cDNA using the QuantiTect Reverse Transcription Kit (both from Qiagen, Hilden, Germany). Multiplexed ddPCR was performed using a QX200 ddPCR system (Bio-Rad Laboratories, Hercules, Calif., USA) with probes targeting KH902 and the reference transcript, glucuronidase beta (GUSB) (#4448489; ThermoFisher). Primer and probe sets for KH902 were designed and synthesized by Integrated DNA Technologies (Coralville, Iowa, USA) (forward: 5′-GGACATACACAACCAGAGAGAC-3′ (SEQ ID NO: 27) and reverse: 5′-GTGAGTGAAAGAGACACAGGAA-3 (SEQ ID NO: 28), probe: 5′-/56-FAM/CCCATTTCA/ZEN/AAGGAGAAGCAGAGCCA/3IABkfq/-3′ (SEQ ID NO: 29)). KH902 mRNA copy number was normalized to GUSB copies. The ddPCR results are presented as the ratio of KH902 values to GUSB values.

Western Blot

Total protein from pooled corneas in each group was extracted on ice in RIPA lysis buffer with fresh protease and phosphatase inhibitors (Thermo Fisher Scientific, Waltham, Mass., USA), following homogenization using QIAGEN TissueLysis II. A total of 20 μg/lane protein extract was loaded onto a 4%-12% Bis-Tris Precast Gel (QP3510, SMOBIO) and transferred onto polyvinylidene difluoride (PVDF) membranes (Millipore). Nonspecific binding was blocked with 5% BSA in Tris-buffered saline with Tween-20 (TBST). Membranes were incubated with rabbit anti-Cleaved Notchl (#4147, Cell signaling Technology), goat anti-mouse D114 (AF1389, R&D Systems), rabbit anti-pERK1/2(#4370, Cell signaling Technology) and anti-ERK1/2(#9102, Cell signaling Technology) antibodies overnight at 4° C. Membranes were incubated with rabbit anti-ERK following membrane harsh stripping. After washing with TBST, the membranes were incubated with horseradish peroxidase-conjugated goat anti-rabbit IgG (1:10,000, G-21234; Invitrogen), or rabbit anti-goat IgG (1:1000; HAF017, R&D Systems) for one and half hours. Protein detection was performed using the Enhanced Chemiluminescence (ECL) Western Blotting Substrate (cat. no. W1001; Promega, Madison, Wis., USA) in combination with the Odyssey system. The intensity of the specific bands was quantified using ImageJ software.

Statistics

Results are expressed as mean±SEM. Each data point represents the mean of 3 replicate values. Analysis was performed using one-way or two-way ANOVA for multiple variables, and Tukey's multiple-comparison test was used for inter-group differences using GraphPad Prism 7.0 (GraphPad Software, La Jolla, Calif., USA). p<0.05 was considered significant.

### Example 5: Plasmid Analysis

Two plasmids containing the KH902 transgene were produced. Plasmid 1 comprises an rAAV vector comprising a 5′AAV ITR, a CBA promoter, an intron, a Kozak sequence, a transgene encoding an KH902, a rabbit globulin polyA, and a 3′AAV ITR. The rAAV vector sequence runs from the 5′-ITR to the 3′-ITR of Plasmid 1 and is set forth in SEQ ID NO: 3. The entire plasmid sequence of Plasmid 1 is set forth in SEQ ID NO:30.

Plasmid 2 comprises an rAAV vector comprising a 5′AAV ITR, a CMV promoter, an intron, a Kozak sequence, a transgene encoding an KH902, a SV40 polyA, and a 3′AAV ITR. The sequence of the KH902 transgene was codon optimized. The entire plasmid sequence of Plasmid 2 is set forth in SEQ ID NO: 31.

There are two ITR sequences in each of the plasmids, named ITR1 and ITR2 (e.g., the 5′ and 3′ ITRs of the rAAV vector, respectively). There are multiple SmaI sites in the two plasmids; two in ITR1 and two in ITR2. The number and size of theoretical DNA bands after plasmid digestion with SmaI was calculated. When the plasmid is intact, the position of SmaI digestion can be determined according to the DNA sequence, and the number and size of DNA bands can be calculated after being fully digested by SmaI. This is the band profile of the intact plasmid. When the ITR1 of the plasmid is digested (equivalent to the deletion of the SmaI sites in ITR1), the number and size of the DNA bands of the deleted plasmid after being fully digested with SmaI was calculated. This is the band profile of the ITR1-deleted plasmid. The same method was used to calculate the band profile of ITR2-deleted plasmid, and ITR1+ITR2-deleted plasmid after being fully digested with SmaI. Samples of Plasmid 1 and Plasmid 2 were fully digested using SmaI, and agarose gel electrophoresis were performed. In the case of ITR deletions, the sample is expected to be a mixture of intact plasmid and deleted plasmid. According to the electrophoretic DNA bands, the possible type and degree of ITR deletion can be estimated.

If Plasmid 1 is intact, the gel electrophoresis spectrum will show bands near 407 bp, 307 bp, 343 bp, 2868 bp, and 2817 bp. When the ITR1 of plasmid 1 is deleted, a band will appear near at 3171 bp. If ITR2 is missing, a band will appear near at 5696 bp, and if ITR1 and ITR2 both are missing, a band will appear near at 6050 bp. The experimental results showed that the gel electrophoresis spectrum of Plasmid 1 digestion was consistent with the theoretical complete plasmid spectrum, and there was no band around 3171, 5696, or 6050 bp, indicating that Plasmid 1 had no ITR deletions.

If the Plasmid 2 is intact, the gel electrophoresis complete spectrum will show bands near 2817 bp and 2834 bp. When ITR1 and/or ITR2 are missing, a band will appear at 5673 bp. The gel electrophoresis results of Plasmid 2 digestion showed that there were a few bands >5000 bp, while the normal plasmid digestion map showed no bands above 5000 bp, indicating that Plasmid 2 comprises ITR1 and/or ITR2 deletions.

### Example 6: Protein Expression after a Single Intravitreal Injection in Cyanotic Blue Rabbits

This example describes the distribution of KH902 protein expressed by different rAAV vectors in the eye tissues of cyanotic blue rabbits after a single intravitreal injection. An rAAV 7m8-CBA-KH902, which contains the KH902 transgene driven by the CMV enhancer and chicken β-actin promoter regulatory cassette (e.g., Plasmid 1 as describe in Example 5) encapsidated by an AAV7m8 capsid protein, was used. An rAAV 7m8-CMV-KH902, which contains the isolated nucleic acid comprising CMV promoter, an intron, a Kozak sequence, a codon optimized transgene encoding an KH902, a WPRE, a SV40 polyA, and a 3′AAV ITR encapsidated by an AAV7m8 capsid protein, was used for comparison. The plasmid used to produce rAAV7m8-CMV-902 is set forth in SEQ ID NO: 32).

After intravitreal injection of rAAV 7m8-CBA-KH902 or rAAV 7m8-CMV-KH902 in the left and right eyes of cyanotic blue rabbits (2E¹¹ vg/50₁11), the animals were sacrificed at the specified time (Week 2 and Week 4). Then the eyeballs and optic nerves were removed, the eyeballs were dissected, and the retinal-choroid and the vitreous were separated and the expression of Conbercept protein (e.g. KH902 protein) was measured after homogenization.

Data indicate that rAAV 7m8-CBA-KH902 had higher and more stable expression in retinal choroidal plexus and vitreous when compared with rAAV 7m8-CMV-KH902 (Table 1).

TABLE 1

| | | retinal-choroid (ng/g) | | | | vitreous (ng/g) | | | |
|---|---|---|---|---|---|---|---|---|---|
| | Sampling | Female- | Female- | Male- | Male- | Female- | Female- | Male- | Male- |
| Vector | time | left | right | left | right | left | right | left | right |
| rAAV 7m8- | week 2 | 1125 | 1432 | 1346 | 207 | 311 | 331 | 291 | 462 |
| CBA-KH902 | week 4 | 1312 | 1763 | 1226 | 208 | 675 | 777 | 193 | 341 |
| rAAV 7m8- | week 2 | 0 | 225 | 3226 | 2900 | 9 | 14 | 12 | 5 |
| CMV-KH902 | week 4 | 86 | 120 | 49 | 19 | 1 | 1 | 3 | 2 |

### Example 7: Expression of Aqueous Humor by Subretinal Delivery in Cynomolgus Monkeys

An rAAV8-CBA-KH902, which contains the KH902 transgene driven by the CMV enhancer, chicken β-action promoter regulatory cassette (e.g., Plasmid 1 as describe in Example 5) was used in this study.

The rAAV8-CBA-KH902 were subretinally injected into eyes of cynomolgus monkeys on the temporal side, just below the superior vascular arch, at a dose of 1E¹² vg/100 μL/eye. The aqueous humor of the anterior chamber was sampled on the 3rd, 7th, 21st and 28th day after administration, about 50 μL/eye. The concentration of target protein in the aqueous humor was detected by ELISA, and the results are shown in Table 2. It was observed that the concentration of Conbercept protein (e.g., KH902 protein) in aqueous humor gradually increased within 28 days after injection.

TABLE 2

aqueous humor (ng/ml)

| Time | left eye | right eye |
|---|---|---|
| Day 3 | Not detected | 0.6 |
| Day 7 | 14.5 | 30.5 |
| Day 21 | 78.6 | 141.4 |
| Day 28 | 116 | 162.6 |

### Example 8: Efficacy of Vectors in Rhesus Monkeys by Subretinal Delivery

Nonhuman primates (NHP) have similar macular structure to that of the human. The NHP choroidal neovascularization model induced by laser photocoagulation is one model for nAMD (Wang Q, et al. British Journal of Ophthalmology, 2015,99(1):119-24.). The rAAV8-CBA-KH902 described in Example 7 was used in this study. Rhesus monkeys with healthy eyes were selected to lie supine on the operating table after pupil dilation and anesthesia. The skin around the eyes was disinfected with povidone iodine, and the conjunctival sac was washed with povidone iodine mucosal disinfectant. A WPI microinjection needle (36G) was used to penetrate into the vitreous cavity through the stoma, and a retinoscope was placed on the cornea. The injection was performed near the upper vascular arch of the posterior pole at a volume of 100 μL/eye. The day of administration was recorded as day 1. Dosage and grouping are shown in the table 3 below.

TABLE 3

| | | Number of | Number of | Dose | Volume | |
|---|---|---|---|---|---|---|
| Group No. | Group Name | animals | eyes | vg/eye | μl | Administration Way |
| 1 | High Dose | 3 | 6 | 2E12 | 100 | Subretinal injection |
| 2 | Medium Dose | 3 | 6 | 1E12 | 100 | Subretinal injection |
| 3 | Low Dose | 3 | 6 | 5E11 | 100 | Subretinal injection |
| 4 | Negative control | 3 | 6 | buffer | 100 | Subretinal injection |
| 5 | Positive control | 2 | 4 | 0.5 mg | 50 μL | Intravitreal injection |

On the 15th day, rhesus monkeys were dilated and anesthetized, Carbomer Eye Drops were applied to the ocular surface, and a fundus laser lens was used. After the fundus was clearly seen, the location about 1.5˜2 PD away from the macular fovea was selected to perform photocoagulation avoiding blood vessels. The laser parameters are set as follows: wavelength 532 nm, power 450-550MW, spot diameter 50 μm, exposure time 100 ms. In the positive control group, 50 μL Conbercept Ophthalmic Injection (0.5 mg/eye) was injected intravitreally immediately after laser photocoagulation.

For the detection and inspection index, Enhanced depth imaging optical coherence tomography (EDI-OCT) was used to examine the eyes of animals in group 1-4 before administration, immediately after administration, on day 15 (before and after modeling), on day 29, on day 43, and on day 57. OCT was used to examine the eyes of animals in group 5 before administration, on day 15 (after modeling), on day 29, on day 43, and on day 57. The pre-laser inspection area should cover the back pole, the administration area, and all the laser photocoagulation spots.

The thickness of hyper-reflective material (SHRM) in the OCT image corresponding to grade 4 lesion caused by fluorescent leakage was measured using the software provided by the instrument, and the mean SHRM thickness for each eye was calculated.

Fundus photography and Fluorescent angiography (FP and FFA) were used to examine the eyes of animals in group 1-4 before administration, immediately after administration (FP only), on day 15 (FP only, before and after modeling), on day 29, 43, and 57. FA and FFA was used to examine the eyes of animals in group 5 on day 15 (FP only, after modeling), day 29, day 43, and day 57. Before fundus fluorescein angiography, the animals were intravenously injected with fluorescein sodium injection (10 mg/kg, 100 mg/mL).

The early and late images of fundus fluorescein angiography were compared, and choroidal neovascularization and leakage were determined according to the presence of fluorescence leakage in the fundus of the animals. The degree of fluorescence leakage was rated, and the number and rate of grade 4 lesion were calculated. The rating criteria are shown below:

Grade 1: no high fluorescence appears in the lesion;

Grade 2: lesions with high fluorescence but no fluorescence leakage;

Grade 3: high fluorescence lesion with slight fluorescence leakage, The leakage does not exceed the lesion edge;

Grade 4: high fluorescence lesion with slight fluorescence leakage, The leakage beyond spot edge.

The leakage area of grade 4 lesion should be measured (Note: If the photocoagulation spot is rated as grade 4 lesion at one of the inspection time points after drug administration, the fluorescence leakage area of this lesion should be measured at all inspection time points. If there is no fluorescence leakage, no measurement is required).

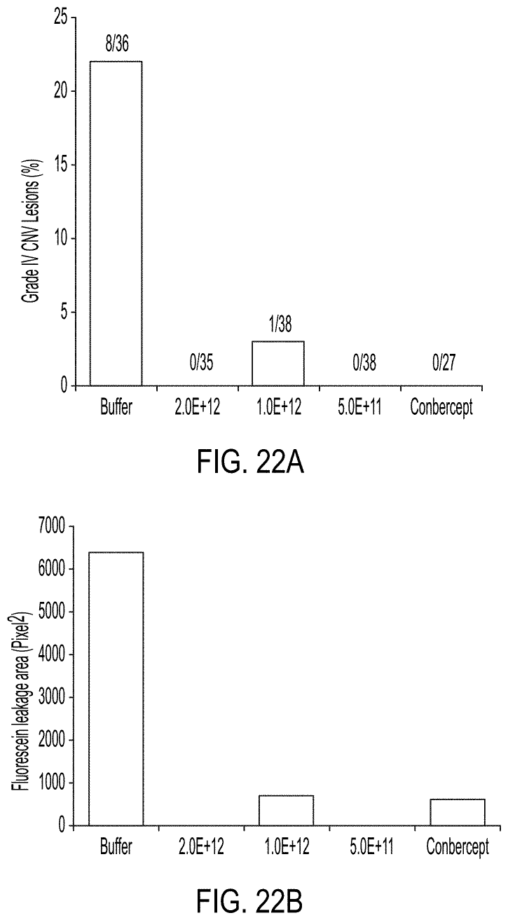

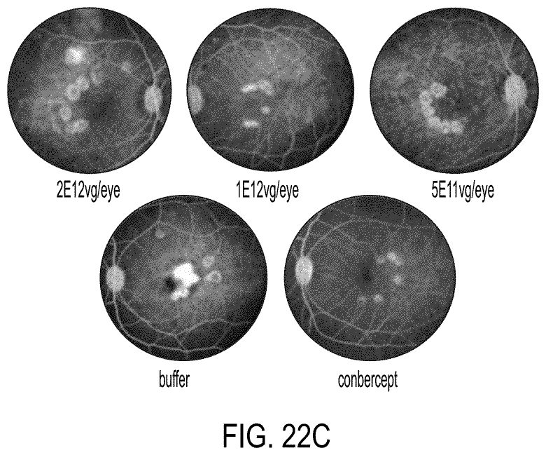

The ratio of grade 4 lesion and the leakage area of grade 4 lesion on the 29th day after administration (i.e. 14 days after modeling) were respectively shown in FIG. 22A and FIG. 22B. It could be seen that the ratio of grade 4 lesion and the leakage area of grade 4 lesion in the high, medium and low dose groups decreased significantly compared with the negative group.

In addition, the regression of the light spot on the 29th day after administration can also be observed from the FFA in FIG. 22C. This figure is a representative FFA diagram of each experimental group. As can be seen from FIG. 22C, the grade 4 lesion of the high, medium and low dose drug groups significantly subsided compared with the negative group.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A recombinant adeno-associated virus (rAAV), comprising:
(i) an AAV capsid protein, wherein the capsid protein is an AAV2 capsid protein, an AAV2/3 hybrid capsid protein, an AAV8 capsid protein or a variant thereof; and
(ii) an isolated nucleic acid comprising a transgene encoding an anti-vascular endothelial growth factor (anti-VEGF) agent, the transgene being flanked by adeno-associated virus (AAV) inverted terminal repeats (ITRs).

2.-42. (canceled)

43. A method of inhibiting VEGF or PlGF activity in a subject in need thereof, the method comprising
administering to the subject an effective amount of the rAAV of claim 1.

44. A method of delivering an anti-VEGF agent in a subject in need thereof, the method comprising administering to the subject an effective amount of the rAAV of claim 1.

45. A method of treating a neovascularization associated disease, an angiogenesis associated disease or a VEGF associated disease in a subject in need thereof, the method comprising
administering to the subject an effective amount of the rAAV of claim 1.

46.-69. (canceled)
